(19) United States
(12) Patent Application Publication
(10) Pub. No.: US 2024/0058339 A1
(43) Pub. Date: Feb. 22, 2024

(54) COMBINATION THERAPY FOR CANCERS WITH KRAS MUTATION

(71) Applicant: COTHERA BIOSCIENCE, INC., (US)

(72) Inventors: Yiyou CHEN, Beijing (CN); Chun JIANG, Hillsborough, CA (US)

(73) Assignee: COTHERA BIOSCIENCE, INC., Grand Cayman (KY)

(21) Appl. No.: 17/766,726

(22) PCT Filed: Oct. 8, 2020
(86) PCT No.: PCT/CN2020/119874

§ 371 Date: Apr. 5, 2022

(30) Foreign Application Priority Data
Oct. 9, 2019 (WO) PCTCN2019110113

(51) Int. Cl. A61K 31/519; A61K 31/4184; A61K 31/4523; A61K 31/506; A61K 39/395; A61P 35/00

(52) U.S. Cl. CPC A61K 31/519; A61K 31/4184; A61K 31/4523; A61K 31/506; A61K 39/3955; A61P 35/00

(57) ABSTRACT

It relates to a combination therapy for treating cancer with KRAS mutations comprising administrating to a subject an effective amount of (a) an epidermal growth factor receptor (EGFR) inhibitor, (b) a mitogen-activated protein kinase (MEK) 1/2 inhibitor, and (c) a cyclin dependent kinase (CDK) 4/6 inhibitor. Also provided are compositions and kits related to the combination therapy.

### CROSS REFERENCE TO RELATED APPLICATION

This application claims the benefit of International Application No. PCT/CN2019/110113, filed Oct. 9, 2019, which is hereby incorporated by reference in its entirety.

### FIELD OF INVENTION

The present disclosure relates generally to combination therapies for treating or delaying progression of cancers with a KRAS mutation and related compositions and kits.

### BACKGROUND

The KRAS is part of the RAS/MAPK signaling pathway, which is involved in the regulation of cell proliferation, survival and differentiation. The KRAS protein is a GTPase which cycles between inactive guanosine diphosphate (GDP)-bound and active guanosine triphosphate (GTP)-bound forms. KRAS has been implicated in the pathogenesis of several cancers, including but not limited to, lung cancer, colorectal cancer and pancreatic cancer. Colorectal cancer (CRC) is the third most common cancer worldwide and the fourth most common cancer in the USA with ˜51,020 deaths expected in 2019. KRAS is the most frequently mutated oncogene in cancer and KRAS mutation is implicated in about 40% of CRC. Nearly all of the KRAS gene mutations associated with CRC change the amino acid glycine at position 12 or 13 (Gly12 or Gly13). G12C, G12V and G13D are among the major KRAS mutations found in CRC. KRAS is implicated in about 40% of all colorectal (CRC) cancers. G12C, G12D and G12V are among the major KRAS mutations found in CRC, accounting for 7%, 28% and 20%, respectively. See EBioMedicine. 2019 March; 41: 711-716. There is currently no approved target therapy to treat CRC harboring KRAS mutations (KRAS CRC). See J Exp Clin Cancer Res. 2018; 37: 57. In addition, current KRAS inhibitors mainly target the KRAS G12C mutations (see, e.g. Nature Reviews Drug Discovery 18, 887-891 (2019)). However, KRAS G12C only account for a small portion of all possible KRAS mutations in cancer patients. See, Nat Rev Drug Discov. 2014; 13(11):828-851. Therefore, there remains a need for a robust pan-KRAS therapy for treating cancer (e.g., colorectal cancer) with KRAS mutations.

The disclosures of all publications, patents, patent applications and published patent applications referred to herein are hereby incorporated herein by reference in their entirety.

### BRIEF SUMMARY

Provided herein are methods for treating or delaying progression of cancer in a subject comprising administering to the subject an effective amount of (a) an epidermal growth factor receptor (EGFR) inhibitor; (b) a mitogen-activated protein kinase (MEK) 1/2 inhibitor; and (c) a cyclin dependent kinase (CDK) 4/6 inhibitor, wherein the subject has cancer that has a KRAS mutation or is at risk of developing cancer that has a KRAS mutation. In some embodiments, the method does not comprise administering a KRAS inhibitor to the subject during the administrations of (a) an epidermal growth factor receptor (EGFR) inhibitor; (b) a mitogen-activated protein kinase (MEK) 1/2 inhibitor; and (c) a cyclin dependent kinase (CDK) 4/6 inhibitor. In some embodiments, the method does not comprise administering an additional therapeutic agent to the subject during the administrations of (a) an epidermal growth factor receptor (EGFR) inhibitor; (b) a mitogen-activated protein kinase (MEK) 1/2 inhibitor; and (c) a cyclin dependent kinase (CDK) 4/6 inhibitor. In some embodiments, the cancer is colorectal cancer (CRC). In some embodiments, the composition comprises osimertinib or a salt thereof, TAK-733 or a salt thereof and palbociclib or a salt thereof, wherein the composition does not comprises a KRAS inhibitor. In some embodiments, the composition comprises cetuximab, TAK-733 or a salt thereof and palbociclib or a salt thereof, wherein the composition does not comprises a KRAS inhibitor. In some embodiments, the composition comprises cetuximab, cobimetinib or a salt thereof and palbociclib or a salt thereof, wherein the composition does not comprises a KRAS inhibitor. In some embodiments, the composition comprises lapatinib or a salt thereof, trametinib or a salt thereof, and palbociclib or a salt thereof, wherein the composition does not comprises a KRAS inhibitor. In some embodiments, the composition comprises osimertinib or a salt thereof, binimetinib or a salt thereof, and palbociclib or a salt thereof, wherein the composition does not comprises a KRAS inhibitor. In some embodiments, the composition comprises cetuximab, binimetinib or a salt thereof, and palbociclib or a salt thereof, wherein the composition does not comprises a KRAS inhibitor. In some embodiments, the composition comprises cetuximab, cobimetinib or a salt thereof, and abemaciclib or a salt thereof, wherein the composition does not comprises a KRAS inhibitor. In some embodiments, the composition consists of osimertinib or a salt thereof, cobimetinib or a salt thereof and palbociclib or a salt thereof. In some embodiments, the composition consists of cetuximab, cobimetinib or a salt thereof and palbociclib or a salt thereof. In some embodiments, the composition consists of osimertinib or a salt thereof, TAK-733 or a salt thereof and palbociclib or a salt thereof. In some embodiments, the composition consists of cetuximab, TAK-733 or a salt thereof and palbociclib or a salt thereof. In some embodiments, the composition consists of lapatinib or a salt thereof, trametinib or a salt thereof, and palbociclib or a salt thereof. In some embodiments, the composition consists of osimertinib or a salt thereof, binimetinib or a salt thereof, and palbociclib or a salt thereof. In some embodiments, the composition consists of cetuximab, binimetinib or a salt thereof, and palbociclib or a salt thereof. In some embodiments, the composition consists of cetuximab, cobimetinib or a salt thereof, and abemaciclib or a salt thereof. In some embodiments, the composition further comprises a pharmaceutically acceptable carrier, excipient, binder, or diluent. In some embodiments, the composition is formulated for oral administration to a subject.

Also provided here are methods for treating or delaying progression of cancer in a subject comprising administering to the subject an effective amount of osimertinib or a salt thereof, cobimetinib or a salt thereof, and palbociclib or a salt thereof, wherein the subject has cancer that has a KRAS mutation or is at risk of developing cancer that has a KRAS mutation. In some embodiments, the method does not comprise administering to the subject a KRAS inhibitor during the administrations of osimertinib or a salt thereof, cobimetinib or a salt thereof, and palbociclib or a salt thereof. In some embodiments, the method does not comprise administering to the subject an additional therapeutic agent during the administrations of osimertinib or a salt thereof, cobimetinib or a salt thereof, and palbociclib or a salt thereof. In some embodiments, osimertinib or a salt thereof, cobimetinib or a salt thereof, and palbociclib or a salt thereof are administered in one composition. In some embodiments, cobimetinib or a salt thereof, and palbociclib or a salt thereof are administered in two or more compositions. In some embodiments, osimertinib or a salt thereof, cobimetinib or a salt thereof, and palbociclib or a salt thereof are administered continuously to the subject. In some embodiments, osimertinib or a salt thereof, cobimetinib or a salt thereof, and palbociclib or a salt thereof are administered intermittently to the subject. In some embodiments, the cancer is CRC.

In some embodiments, osimertinib or a salt thereof is administered to the subject in a daily dose of about 40-160 mg. In some embodiments, cobimetinib or a salt thereof is administered to the subject in a daily dose of about 20-60 mg. In some embodiments, palbociclib or a salt thereof is administered to the subject in a daily dose of about 15-125 mg. In some embodiments, osimertinib or a salt thereof is administered to the subject in a daily dose of about 0.5-3 mg/kg. In some embodiments, cobimetinib or a salt thereof is administered to the subject in a daily dose of about 0.25-1 mg/kg. In some embodiments, palbociclib or a salt thereof is administered to the subject in a daily dose of about 0.25-2.5 mg/kg.

Also provided here are methods for treating or delaying progression of cancer in a subject comprising administering to the subject an effective amount of cetuximab, cobimetinib or a salt thereof, and palbociclib or a salt thereof, wherein the subject has cancer that has a KRAS mutation or is at risk of developing cancer that has a KRAS mutation. In some embodiments, the method does not comprise administering to the subject a KRAS inhibitor during the administrations of cetuximab, cobimetinib or a salt thereof, and palbociclib or a salt thereof. In some embodiments, the method does not comprise administering to the subject an additional therapeutic agent during the administrations of cetuximab, cobimetinib or a salt thereof, and palbociclib or a salt thereof. In some embodiments, cetuximab, cobimetinib or a salt thereof, and palbociclib or a salt thereof are administered in one composition. In some embodiments, cetuximab, and palbociclib or a salt thereof are administered in two or more compositions. In some embodiments, cetuximab, cobimetinib or a salt thereof, and palbociclib or a salt thereof are administered continuously to the subject. In some embodiments, cetuximab, cobimetinib or a salt thereof, and palbociclib or a salt thereof are administered intermittently to the subject. In some embodiments, the cancer is CRC.

In some embodiments, cetuximab is administered to the subject in 400 mg/m² infused over 120 minutes followed by 250 mg/m² weekly infused over 60 minutes. In some embodiments, the maximum infusion rate is about 10 mL/min. In some embodiments, cobimetinib or a salt thereof is administered to the subject in a daily dose of about 20-60 mg. In some embodiments, palbociclib or a salt thereof is administered to the subject in a daily dose of about 15-125 mg. In some embodiments, cetuximab is administered to the subject in a weekly dose of about 150-400 mg/m² per subject. In some embodiments, cobimetinib or a salt thereof is administered to the subject in a daily dose of about 0.25-10 mg/kg. In some embodiments, palbociclib or a salt thereof is administered to the subject in a daily dose of about 0.25-2.5 mg/kg.

Also provided here are methods for treating or delaying progression of cancer in a subject comprising administering to the subject an effective amount of cetuximab, TAK-733 or a salt thereof, and palbociclib or a salt thereof, wherein the subject has cancer that has a KRAS mutation or is at risk of developing cancer that has a KRAS mutation. In some embodiments, the method does not comprise administering to the subject a KRAS inhibitor during the administrations of cetuximab, TAK-733 or a salt thereof, and palbociclib or a salt thereof. In some embodiments, the method does not comprise administering to the subject an additional therapeutic agent during the administrations of cetuximab, TAK-733 or a salt thereof, and palbociclib or a salt thereof. In some embodiments, cetuximab, TAK-733 or a salt thereof, and palbociclib or a salt thereof are administered in one composition. In some embodiments, cetuximab, TAK-733 or a salt thereof and palbociclib or a salt thereof are administered in two or more compositions. In some embodiments, cetuximab, TAK-733 or a salt thereof, and palbociclib or a salt thereof are administered continuously to the subject. In some embodiments, cetuximab, TAK-733 or a salt thereof, and palbociclib or a salt thereof are administered intermittently to the subject. In some embodiments, the cancer is CRC.

In some embodiments, cetuximab is administered to the subject in 400 mg/m² infused over 120 minutes followed by 250 mg/m² weekly infused over 60 minutes. In some embodiments, the maximum infusion rate is about 10 mL/min. In some embodiments, TAK-733 or a salt thereof or a salt thereof is administered to the subject in a daily dose of about 20-60 mg. In some embodiments, palbociclib or a salt thereof is administered to the subject in a daily dose of about 15-125 mg. In some embodiments, cetuximab is administered to the subject in a weekly dose of about 150-400 mg/m² per subject. In some embodiments, TAK-733 or a salt thereof is administered to the subject in a daily dose of about 0.25-30 mg/kg. In some embodiments, palbociclib or a salt thereof is administered to the subject in a daily dose of about 0.25-2.5 mg/kg.

Also provided here are methods for treating or delaying progression of cancer in a subject comprising administering to the subject an effective amount of osimertinib or a salt thereof, TAK-733 or a salt thereof, and palbociclib or a salt thereof, wherein the subject has cancer that has a KRAS mutation or is at risk of developing cancer that has a KRAS mutation. In some embodiments, the method does not comprise administering to the subject a KRAS inhibitor during the administrations of osimertinib or a salt thereof, TAK-733 or a salt thereof, and palbociclib or a salt thereof. In some embodiments, the method does not comprise administering to the subject an additional therapeutic agent during the administrations of osimertinib or a salt thereof, TAK-733 or a salt thereof, and palbociclib or a salt thereof. In some embodiments, osimertinib or a salt thereof, TAK-733 or a salt thereof, and palbociclib or a salt thereof are administered in one composition. In some embodiments, osimertinib or a salt thereof, TAK-733 or a salt thereof and palbociclib or a salt thereof are administered in two or more compositions. In some embodiments, osimertinib or a salt thereof, TAK-733 or a salt thereof, and palbociclib or a salt thereof are administered continuously to the subject. In some embodiments, osimertinib or a salt thereof, TAK-733 or a salt thereof, and palbociclib or a salt thereof are administered intermittently to the subject. In some embodiments, the cancer is CRC.

In some embodiments, osimertinib or a salt thereof is administered to the subject in a daily dose of about 40-160 mg. In some embodiments, TAK-733 or a salt thereof or a salt thereof is administered to the subject in a daily dose of about 20-60 mg. In some embodiments, palbociclib or a salt thereof is administered to the subject in a daily dose of about 15-125 mg. In some embodiments, osimertinib or a salt thereof is administered to the subject in a daily dose of about 0.5-3 mg/kg. In some embodiments, TAK-733 or a salt thereof is administered to the subject in a daily dose of about 0.25-30 mg/kg. In some embodiments, palbociclib or a salt thereof is administered to the subject in a daily dose of about 0.25-2.5 mg/kg.

Also provided here are methods for treating or delaying progression of cancer in a subject comprising administering to the subject an effective amount of osimertinib or a salt thereof, binimetinib or a salt thereof, and palbociclib or a salt thereof, wherein the subject has cancer that has a KRAS mutation or is at risk of developing cancer that has a KRAS mutation. In some embodiments, the method does not comprise administering to the subject a KRAS inhibitor during the administrations of osimertinib or a salt thereof, binimetinib or a salt thereof, and palbociclib or a salt thereof. In some embodiments, the method does not comprise administering to the subject an additional therapeutic agent during the administrations of osimertinib or a salt thereof, binimetinib or a salt thereof, and palbociclib or a salt thereof. In some embodiments, osimertinib or a salt thereof, binimetinib or a salt thereof, and palbociclib or a salt thereof are administered in one composition. In some embodiments, osimertinib or a salt thereof, binimetinib or a salt thereof and palbociclib or a salt thereof are administered in two or more compositions (e.g., two or three compositions). In some embodiments, osimertinib or a salt thereof, binimetinib or a salt thereof, and palbociclib or a salt thereof are administered continuously to the subject. In some embodiments, osimertinib or a salt thereof, binimetinib or a salt thereof, and palbociclib or a salt thereof are administered intermittently to the subject. In some embodiments, the cancer is CRC.

In some embodiments, osimertinib or a salt thereof is administered to the subject in a daily dose of about 40-160 mg. In some embodiments, binimetinib or a salt thereof or a salt thereof is administered to the subject in a daily dose of about 60-90 mg. In some embodiments, palbociclib or a salt thereof is administered to the subject in a daily dose of about 15-125 mg. In some embodiments, osimertinib or a salt thereof is administered to the subject in a daily dose of about 0.5-3 mg/kg. In some embodiments, binimetinib or a salt thereof is administered to the subject in a daily dose of about 0.8-2.7 mg/kg. In some embodiments, palbociclib or a salt thereof is administered to the subject in a daily dose of about 0.25-2.5 mg/kg.

Also provided here are methods for treating or delaying progression of cancer in a subject comprising administering to the subject an effective amount of cetuximab, binimetinib or a salt thereof, and palbociclib or a salt thereof, wherein the subject has cancer that has a KRAS mutation or is at risk of developing cancer that has a KRAS mutation. In some embodiments, the method does not comprise administering to the subject a KRAS inhibitor during the administrations of cetuximab, binimetinib or a salt thereof, and palbociclib or a salt thereof. In some embodiments, the method does not comprise administering to the subject an additional therapeutic agent during the administrations of cetuximab, binimetinib or a salt thereof, and palbociclib or a salt thereof. In some embodiments, cetuximab, binimetinib or a salt thereof, and palbociclib or a salt thereof are administered in one composition. In some embodiments, cetuximab, binimetinib or a salt thereof and palbociclib or a salt thereof are administered in two or more compositions (e.g., two or three compositions). In some embodiments, cetuximab, binimetinib or a salt thereof, and palbociclib or a salt thereof are administered continuously to the subject. In some embodiments, cetuximab, binimetinib or a salt thereof, and palbociclib or a salt thereof are administered intermittently to the subject. In some embodiments, the cancer is CRC.

In some embodiments, cetuximab is administered to the subject in 400 mg/m² infused over 120 minutes followed by 250 mg/m² weekly infused over 60 minutes. In some embodiments, the maximum infusion rate is about 10 mL/min. In some embodiments, binimetinib or a salt thereof or a salt thereof is administered to the subject in a daily dose of about 60-90 mg. In some embodiments, palbociclib or a salt thereof is administered to the subject in a daily dose of about 15-125 mg. In some embodiments, cetuximab is administered to the subject in a daily dose of about 0.5-3 mg/kg. In some embodiments, binimetinib or a salt thereof is administered to the subject in a daily dose of about 0.8-2.7 mg/kg. In some embodiments, palbociclib or a salt thereof is administered to the subject in a daily dose of about 0.25-2.5 mg/kg.

Also provided here are methods for treating or delaying progression of cancer in a subject comprising administering to the subject an effective amount of cetuximab, cobimetinib or a salt thereof, and abemaciclib or a salt thereof, wherein the subject has cancer that has a KRAS mutation or is at risk of developing cancer that has a KRAS mutation. In some embodiments, the method does not comprise administering to the subject a KRAS inhibitor during the administrations of cetuximab, cobimetinib or a salt thereof, and abemaciclib or a salt thereof. In some embodiments, the method does not comprise administering to the subject an additional therapeutic agent during the administrations of cetuximab, cobimetinib or a salt thereof, and abemaciclib or a salt thereof. In some embodiments, cetuximab, cobimetinib or a salt thereof, and abemaciclib or a salt thereof are administered in one composition. In some embodiments, cetuximab, and abemaciclib or a salt thereof are administered in two or more compositions. In some embodiments, cetuximab, cobimetinib or a salt thereof, and abemaciclib or a salt thereof are administered continuously to the subject. In some embodiments, cetuximab, cobimetinib or a salt thereof, and abemaciclib or a salt thereof are administered intermittently to the subject. In some embodiments, the cancer is CRC.

In some embodiments, cetuximab is administered to the subject in 400 mg/m² infused over 120 minutes followed by 250 mg/m² weekly infused over 60 minutes. In some embodiments, the maximum infusion rate is about 10 mL/min. In some embodiments, cobimetinib or a salt thereof is administered to the subject in a daily dose of about 20-60 mg. In some embodiments, abemaciclib or a salt thereof is administered to the subject in a daily dose of about 100-400 mg. In some embodiments, cetuximab is administered to the subject in a weekly dose of about 150-400 mg/m² per subject. In some embodiments, cobimetinib or a salt thereof is administered to the subject in a daily dose of about 0.25-10 mg/kg. In some embodiments, abemaciclib or a salt thereof is administered to the subject in a daily dose of about 4-6 mg/kg.

Also provided here are methods for treating or delaying progression of cancer in a subject comprising administering to the subject an effective amount of lapatinib or a salt thereof, trametinib or a salt thereof, and palbociclib or a salt thereof, wherein the subject has cancer that has a KRAS mutation or is at risk of developing cancer that has a KRAS mutation. In some embodiments, the method does not comprise administering to the subject a KRAS inhibitor during the administrations of lapatinib or a salt thereof, trametinib or a salt thereof, and palbociclib or a salt thereof. In some embodiments, the method does not comprise administering to the subject an additional therapeutic agent during the administrations of lapatinib or a salt thereof, trametinib or a salt thereof, and palbociclib or a salt thereof. In some embodiments, lapatinib or a salt thereof, trametinib or a salt thereof, and palbociclib or a salt thereof are administered in one composition. In some embodiments, lapatinib or a salt thereof, and palbociclib or a salt thereof are administered in two or more compositions. In some embodiments, lapatinib or a salt thereof, trametinib or a salt thereof, and palbociclib or a salt thereof are administered continuously to the subject. In some embodiments, lapatinib or a salt thereof, trametinib or a salt thereof, and palbociclib or a salt thereof are administered intermittently to the subject. In some embodiments, the cancer is CRC.

In some embodiments, lapatinib or a salt thereof is administered to the subject in a daily dose of about 500-5500 mg. In some embodiments, trametinib or a salt thereof is administered to the subject in a daily dose of about 0.5-4 mg. In some embodiments, palbociclib or a salt thereof is administered to the subject in a daily dose of about 15-125 mg. In some embodiments, lapatinib or a salt thereof is administered to the subject in a daily dose of about 6-75 mg/kg. In some embodiments, trametinib or a salt thereof is administered to the subject in a daily dose of about 0.01-0.1 mg/kg. In some embodiments, palbociclib or a salt thereof is administered to the subject in a daily dose of about 0.25-2.5 mg/kg.

Also provided here are methods for treating or delaying progression of cancer in a subject comprising administering to the subject an effective amount of cetuximab, trametinib or a salt thereof, and palbociclib or a salt thereof, wherein the subject has cancer that has a KRAS mutation or is at risk of developing cancer that has a KRAS mutation. In some embodiments, the method does not comprise administering to the subject a KRAS inhibitor during the administrations of cetuximab, trametinib or a salt thereof, and palbociclib or a salt thereof. In some embodiments, the method does not comprise administering to the subject an additional therapeutic agent during the administrations of cetuximab, trametinib or a salt thereof, and palbociclib or a salt thereof. In some embodiments, cetuximab, trametinib or a salt thereof, and palbociclib or a salt thereof are administered in one composition. In some embodiments, cetuximab, and palbociclib or a salt thereof are administered in two or more compositions. In some embodiments, cetuximab, trametinib or a salt thereof, and palbociclib or a salt thereof are administered continuously to the subject. In some embodiments, cetuximab, trametinib or a salt thereof, and palbociclib or a salt thereof are administered intermittently to the subject. In some embodiments, the cancer is CRC.

In some embodiments, cetuximab is administered to the subject in 400 mg/m² infused over 120 minutes followed by 250 mg/m² weekly infused over 60 minutes. In some embodiments, the maximum infusion rate is about 10 mL/min. In some embodiments, trametinib or a salt thereof is administered to the subject in a daily dose of about 0.5-2 mg. In some embodiments, palbociclib or a salt thereof is administered to the subject in a daily dose of about 15-125 mg. In some embodiments, cetuximab is administered to the subject in a weekly dose of about 150-400 mg/m² per subject. In some embodiments, trametinib or a salt thereof is administered to the subject in a daily dose of about 0.25-10 mg/kg. In some embodiments, palbociclib or a salt thereof is administered to the subject in a daily dose of about 0.25-2.5 mg/kg.

Also provided here are methods for treating or delaying progression of cancer in a subject comprising administering to the subject an effective amount of osimertinib or a salt thereof, trametinib or a salt thereof, and palbociclib or a salt thereof, wherein the subject has cancer that has a KRAS mutation or is at risk of developing cancer that has a KRAS mutation. In some embodiments, the method does not comprise administering to the subject a KRAS inhibitor during the administrations of osimertinib or a salt thereof, trametinib or a salt thereof, and palbociclib or a salt thereof. In some embodiments, the method does not comprise administering to the subject an additional therapeutic agent during the administrations of osimertinib or a salt thereof, trametinib or a salt thereof, and palbociclib or a salt thereof. In some embodiments, osimertinib or a salt thereof, trametinib or a salt thereof, and palbociclib or a salt thereof are administered in one composition. In some embodiments, trametinib or a salt thereof, and palbociclib or a salt thereof are administered in two or more compositions. In some embodiments, osimertinib or a salt thereof, trametinib or a salt thereof, and palbociclib or a salt thereof are administered continuously to the subject. In some embodiments, osimertinib or a salt thereof, trametinib or a salt thereof, and palbociclib or a salt thereof are administered intermittently to the subject. In some embodiments, the cancer is CRC.

In some embodiments, osimertinib or a salt thereof is administered to the subject in a daily dose of about 40-160 mg. In some embodiments, trametinib or a salt thereof is administered to the subject in a daily dose of about 0.5-2 mg. In some embodiments, palbociclib or a salt thereof is administered to the subject in a daily dose of about 15-125 mg. In some embodiments, osimertinib or a salt thereof is administered to the subject in a daily dose of about 0.5-3 mg/kg. In some embodiments, trametinib or a salt thereof is administered to the subject in a daily dose of about 0.25-1 mg/kg. In some embodiments, palbociclib or a salt thereof is administered to the subject in a daily dose of about 0.25-2.5 mg/kg.

In some embodiments, the cancer has a KRAS G12 mutation or a KRAS G13 mutation. In some embodiments, the KRAS G13 mutation is KRAS G13D mutation. In some embodiments, the KRAS G12 mutation is KRAS G12C or G12V mutation. In some embodiments, the cancer is a malignant epithelial tumor or carcinoma. In some embodiments, the cancer is a carcinoma selected from one or more of a lung cancer, colorectal cancer and pancreatic cancer. In some embodiments, the cancer is CRC. In some embodiments, the CRC has a KRAS G12C or G12V mutation. In some embodiments, the CRC has a KRAS G13D mutation.

In some embodiments, the subject had received a KRAS inhibitor in a previous treatment cycle. In some embodiments, the subject has not received a KRAS inhibitor in a previous treatment cycle.

In some embodiments, the method provided herein reduces cancer cell growth and/or increase cancer cell-killing by about 20-99% more than administration of (a) an epidermal growth factor receptor (EGFR) inhibitor; (b) a mitogen-activated protein kinase (MEK) 1/2 inhibitor; or (c) a cyclin dependent kinase (CDK) 4/6 inhibitor alone. In some embodiments, the method provided herein reduces cancer cell growth and/or increase cancer cell-killing by about 20-99% more than administration of osimertinib or a salt thereof, cobimetinib or a salt thereof, or palbociclib or a salt thereof alone. In some embodiments, the method provided herein reduces cancer cell growth and/or increase cancer cell-killing by about 20-99% more than administration of cetuximab, cobimetinib or a salt thereof, or palbociclib or a salt thereof alone. In some embodiments, the method provided herein reduces cancer cell growth and/or increase cancer cell-killing by about 20-99% more than administration of cetuximab, TAK-733 or a salt thereof, or palbociclib or a salt thereof alone. In some embodiments, the method provided herein reduces cancer cell growth and/or increase cancer cell-killing by about 20-99% more than administration of osimertinib or a salt thereof, TAK-733 or a salt thereof, or palbociclib or a salt thereof alone. In some embodiments, the method reduces tumor volume by about 20-95%.

In another aspect, provided herein are kits comprising (a) an epidermal growth factor receptor (EGFR) inhibitor; (b) a mitogen-activated protein kinase (MEK) 1/2 inhibitor; and (c) a cyclin dependent kinase (CDK) 4/6 inhibitor; wherein the kit does not comprises a KRAS inhibitor.

In some embodiments, the kit comprises osimertinib or a salt thereof, cobimetinib or a salt thereof, and palbociclib or a salt thereof. In some embodiments, the kit does not comprise a KRAS inhibitor. In some embodiments, the kit comprises a pharmaceutical composition comprising osimertinib or a salt thereof and a pharmaceutically acceptable carrier, excipient, binder, or diluent. In some embodiments, the kit comprises a pharmaceutical composition comprising cobimetinib or a salt thereof and a pharmaceutically acceptable carrier, excipient, binder, or diluent. In some embodiments, the kit comprises a pharmaceutical composition comprising palbociclib or a salt thereof and a pharmaceutically acceptable carrier, excipient, binder, or diluent. In some embodiments, the osimertinib or a salt thereof is formulated for oral administration to a subject. In some embodiments, the cobimetinib or a salt thereof is formulated for oral administration to a subject. In some embodiments, the palbociclib or a salt thereof is formulated for oral administration to a subject.

In some embodiments, osimertinib or a salt thereof, cobimetinib or a salt thereof, and palbociclib or a salt thereof are formulated as one composition. In some embodiments, osimertinib or a salt thereof, cobimetinib or a salt thereof, and palbociclib or a salt thereof are formulated as individual compositions. In some embodiments, osimertinib or a salt thereof and cobimetinib or a salt thereof are formulated as one composition. In some embodiments, osimertinib or a salt thereof and palbociclib or a salt thereof are formulated as one composition. In some embodiments, cobimetinib or a salt thereof and palbociclib or a salt thereof are formulated as one composition. In some embodiments, osimertinib or a salt thereof, cobimetinib or a salt thereof, and palbociclib or a salt thereof are formulated in liquid forms. In some embodiments, osimertinib or a salt thereof, cobimetinib or a salt thereof, and palbociclib or a salt thereof are formulated in solid forms.

In some embodiments, osimertinib or a salt thereof, cobimetinib or a salt thereof, and palbociclib or a salt thereof are administered as one composition. In some embodiments, osimertinib or a salt thereof, cobimetinib or a salt thereof, and palbociclib or a salt thereof are administered separately. In some embodiments, osimertinib or a salt thereof, cobimetinib or a salt thereof, and palbociclib or a salt thereof are administered simultaneously. In some embodiments, osimertinib or a salt thereof, cobimetinib or a salt thereof, and palbociclib or a salt thereof are administered continuously. In some embodiments, osimertinib or a salt thereof, cobimetinib or a salt thereof, and palbociclib or a salt thereof are administered intermittently.

In some embodiments, the kit comprises cetuximab, cobimetinib or a salt thereof, and palbociclib or a salt thereof. In some embodiments, the kit does not comprise a KRAS inhibitor. In some embodiments, the kit comprises a pharmaceutical composition comprising cetuximab and a pharmaceutically acceptable carrier, excipient, binder, or diluent. In some embodiments, the kit comprises a pharmaceutical composition comprising cobimetinib or a salt thereof and a pharmaceutically acceptable carrier, excipient, binder, or diluent. In some embodiments, the kit comprises a pharmaceutical composition comprising palbociclib or a salt thereof and a pharmaceutically acceptable carrier, excipient, binder, or diluent. In some embodiments, cetuximab is formulated for administration to a subject via intravenous infusion. In some embodiments, cobimetinib or a salt thereof is formulated for oral administration to a subject. In some embodiments, palbociclib or a salt thereof is formulated for oral administration to a subject. In some embodiments, cetuximab, cobimetinib or a salt thereof, and palbociclib or a salt thereof are formulated in different compositions. In some embodiments, cobimetinib or a salt thereof, and palbociclib or a salt thereof are formulated in one composition.

In some embodiments, cetuximab, cobimetinib or a salt thereof, and palbociclib or a salt thereof are formulated as individual compositions. In some embodiments, cetuximab and palbociclib or a salt thereof are formulated as one composition. In some embodiments, cobimetinib or a salt thereof and palbociclib or a salt thereof are formulated as one composition. In some embodiments, cetuximab, cobimetinib or a salt thereof, and palbociclib or a salt thereof are formulated in liquid forms. In some embodiments, cetuximab, cobimetinib or a salt thereof, and palbociclib or a salt thereof are formulated in solid forms.

In some embodiments, cetuximab, cobimetinib or a salt thereof, and palbociclib or a salt thereof are administered separately. In some embodiments, cetuximab, cobimetinib or a salt thereof, and palbociclib or a salt thereof are administered simultaneously. In some embodiments, cetuximab, cobimetinib or a salt thereof, and palbociclib or a salt thereof are administered continuously. In some embodiments, cetuximab, cobimetinib or a salt thereof, and palbociclib or a salt thereof are administered intermittently. In some embodiments, cetuximab, cobimetinib or a salt thereof, and palbociclib or a salt thereof are administered with different dosing frequencies.

In some embodiments, the kit comprises cetuximab, TAK-733 or a salt thereof, and palbociclib or a salt thereof. In some embodiments, the kit does not comprise a KRAS inhibitor. In some embodiments, the kit comprises a pharmaceutical composition comprising cetuximab and a pharmaceutically acceptable carrier, excipient, binder, or diluent. In some embodiments, the kit comprises a pharmaceutical composition comprising TAK-733 or a salt thereof and a pharmaceutically acceptable carrier, excipient, binder, or diluent. In some embodiments, the kit comprises a pharmaceutical composition comprising palbociclib or a salt thereof and a pharmaceutically acceptable carrier, excipient, binder, or diluent. In some embodiments, cetuximab is formulated for administration to a subject via intravenous infusion. In some embodiments, TAK-733 or a salt thereof is formulated for oral administration to a subject. In some embodiments, palbociclib or a salt thereof is formulated for oral administration to a subject. In some embodiments, cetuximab, TAK-733 or a salt thereof, and palbociclib or a salt thereof are formulated in different compositions. In some embodiments, TAK-733 or a salt thereof, and palbociclib or a salt thereof are formulated in one composition.

In some embodiments, cetuximab, TAK-733 or a salt thereof, and palbociclib or a salt thereof are formulated as individual compositions. In some embodiments, cetuximab and palbociclib or a salt thereof are formulated as one composition. In some embodiments, TAK-733 or a salt thereof and palbociclib or a salt thereof are formulated as one composition. In some embodiments, cetuximab, TAK-733 or a salt thereof, and palbociclib or a salt thereof are formulated in liquid forms. In some embodiments, cetuximab, TAK-733 or a salt thereof, and palbociclib or a salt thereof are formulated in solid forms.

In some embodiments, cetuximab, TAK-733 or a salt thereof, and palbociclib or a salt thereof are administered separately. In some embodiments, cetuximab, TAK-733 or a salt thereof, and palbociclib or a salt thereof are administered simultaneously. In some embodiments, cetuximab, TAK-733 or a salt thereof, and palbociclib or a salt thereof are administered continuously. In some embodiments, cetuximab, TAK-733 or a salt thereof, and palbociclib or a salt thereof are administered intermittently. In some embodiments, cetuximab, TAK-733 or a salt thereof, and palbociclib or a salt thereof are administered with different dosing frequencies.

In some embodiments, the kit comprises osimertinib or a salt thereof, TAK-733 or a salt thereof, and palbociclib or a salt thereof. In some embodiments, the kit does not comprise a KRAS inhibitor. In some embodiments, the kit comprises a pharmaceutical composition comprising osimertinib or a salt thereof and a pharmaceutically acceptable carrier, excipient, binder, or diluent. In some embodiments, the kit comprises a pharmaceutical composition comprising TAK-733 or a salt thereof and a pharmaceutically acceptable carrier, excipient, binder, or diluent. In some embodiments, the kit comprises a pharmaceutical composition comprising palbociclib or a salt thereof and a pharmaceutically acceptable carrier, excipient, binder, or diluent. In some embodiments, the osimertinib or a salt thereof is formulated for oral administration to a subject. In some embodiments, the TAK-733 or a salt thereof is formulated for oral administration to a subject. In some embodiments, the palbociclib or a salt thereof is formulated for oral administration to a subject.

In some embodiments, osimertinib or a salt thereof, TAK-733 or a salt thereof, and palbociclib or a salt thereof are formulated as one composition. In some embodiments, osimertinib or a salt thereof, TAK-733 or a salt thereof, and palbociclib or a salt thereof are formulated as individual compositions. In some embodiments, osimertinib or a salt thereof and TAK-733 or a salt thereof are formulated as one composition. In some embodiments, osimertinib or a salt thereof and palbociclib or a salt thereof are formulated as one composition. In some embodiments, TAK-733 or a salt thereof and palbociclib or a salt thereof are formulated as one composition. In some embodiments, osimertinib or a salt thereof, TAK-733 or a salt thereof, and palbociclib or a salt thereof are formulated in liquid forms. In some embodiments, osimertinib or a salt thereof, TAK-733 or a salt thereof, and palbociclib or a salt thereof are formulated in solid forms.

In some embodiments, osimertinib or a salt thereof, TAK-733 or a salt thereof, and palbociclib or a salt thereof are administered as one composition. In some embodiments, osimertinib or a salt thereof, TAK-733 or a salt thereof, and palbociclib or a salt thereof are administered separately. In some embodiments, osimertinib or a salt thereof, TAK-733 or a salt thereof, and palbociclib or a salt thereof are administered simultaneously. In some embodiments, osimertinib or a salt thereof, TAK-733 or a salt thereof, and palbociclib or a salt thereof are administered continuously. In some embodiments, osimertinib or a salt thereof, TAK-733 or a salt thereof, and palbociclib or a salt thereof are administered intermittently.

In some embodiments, the kit comprises osimertinib or a salt thereof, binimetinib or a salt thereof, and palbociclib or a salt thereof. In some embodiments, the kit does not comprise a KRAS inhibitor. In some embodiments, the kit comprises a pharmaceutical composition comprising osimertinib and a pharmaceutically acceptable carrier, excipient, binder, or diluent. In some embodiments, the kit comprises a pharmaceutical composition comprising binimetinib or a salt thereof and a pharmaceutically acceptable carrier, excipient, binder, or diluent. In some embodiments, the kit comprises a pharmaceutical composition comprising palbociclib or a salt thereof and a pharmaceutically acceptable carrier, excipient, binder, or diluent. In some embodiments, osimertinib is formulated for oral administration to a subject. In some embodiments, binimetinib or a salt thereof is formulated for oral administration to a subject. In some embodiments, palbociclib or a salt thereof is formulated for oral administration to a subject. In some embodiments, osimertinib or a salt thereof, binimetinib or a salt thereof, and palbociclib or a salt thereof are formulated in different compositions. In some embodiments, osimertinib or a salt thereof, binimetinib or a salt thereof, and palbociclib or a salt thereof are formulated in one composition.

In some embodiments, osimertinib or a salt thereof, binimetinib or a salt thereof, and palbociclib or a salt thereof are formulated as one composition. In some embodiments, osimertinib or a salt thereof, binimetinib or a salt thereof, and palbociclib or a salt thereof are formulated as individual compositions. In some embodiments, osimertinib and palbociclib or a salt thereof are formulated as one composition. In some embodiments, binimetinib or a salt thereof and palbociclib or a salt thereof are formulated as one composition. In some embodiments, osimertinib or a salt thereof, binimetinib or a salt thereof, and palbociclib or a salt thereof are formulated in liquid forms. In some embodiments, osimertinib or a salt thereof, binimetinib or a salt thereof, and palbociclib or a salt thereof are formulated in solid forms.

In some embodiments, osimertinib or a salt thereof, binimetinib or a salt thereof, and palbociclib or a salt thereof are administered as one composition. In some embodiments, osimertinib or a salt thereof, binimetinib or a salt thereof, and palbociclib or a salt thereof are administered separately. In some embodiments, osimertinib or a salt thereof, binimetinib or a salt thereof, and palbociclib or a salt thereof are administered simultaneously. In some embodiments, osimertinib or a salt thereof, binimetinib or a salt thereof, and palbociclib or a salt thereof are administered continuously. In some embodiments, osimertinib or a salt thereof, binimetinib or a salt thereof, and palbociclib or a salt thereof are administered intermittently. In some embodiments, osimertinib or a salt thereof, binimetinib or a salt thereof, and palbociclib or a salt thereof are administered with different dosing frequencies.

In some embodiments, the kit comprises cetuximab, binimetinib or a salt thereof, and palbociclib or a salt thereof. In some embodiments, the kit does not comprise a KRAS inhibitor. In some embodiments, the kit comprises a pharmaceutical composition comprising cetuximab and a pharmaceutically acceptable carrier, excipient, binder, or diluent. In some embodiments, the kit comprises a pharmaceutical composition comprising binimetinib or a salt thereof and a pharmaceutically acceptable carrier, excipient, binder, or diluent. In some embodiments, the kit comprises a pharmaceutical composition comprising palbociclib or a salt thereof and a pharmaceutically acceptable carrier, excipient, binder, or diluent. In some embodiments, cetuximab is formulated for administration to a subject via intravenous infusion. In some embodiments, binimetinib or a salt thereof is formulated for oral administration to a subject. In some embodiments, palbociclib or a salt thereof is formulated for oral administration to a subject. In some embodiments, cetuximab, binimetinib or a salt thereof, and palbociclib or a salt thereof are formulated in different compositions. In some embodiments, binimetinib or a salt thereof, and palbociclib or a salt thereof are formulated in one composition.

In some embodiments, cetuximab, binimetinib or a salt thereof, and palbociclib or a salt thereof are formulated as one composition. In some embodiments, cetuximab, binimetinib or a salt thereof, and palbociclib or a salt thereof are formulated as individual compositions. In some embodiments, cetuximab and palbociclib or a salt thereof are formulated as one composition. In some embodiments, binimetinib or a salt thereof and palbociclib or a salt thereof are formulated as one composition. In some embodiments, cetuximab, binimetinib or a salt thereof, and palbociclib or a salt thereof are formulated in liquid forms. In some embodiments, cetuximab, binimetinib or a salt thereof, and palbociclib or a salt thereof are formulated in solid forms.

In some embodiments, cetuximab, binimetinib or a salt thereof, and palbociclib or a salt thereof are administered separately. In some embodiments, cetuximab, binimetinib or a salt thereof, and palbociclib or a salt thereof are administered simultaneously. In some embodiments, cetuximab, binimetinib or a salt thereof, and palbociclib or a salt thereof are administered continuously. In some embodiments, cetuximab, binimetinib or a salt thereof, and palbociclib or a salt thereof are administered intermittently. In some embodiments, cetuximab, binimetinib or a salt thereof, and palbociclib or a salt thereof are administered with different dosing frequencies.

In some embodiments, the kit comprises cetuximab, trametinib or a salt thereof, and palbociclib or a salt thereof. In some embodiments, the kit does not comprise a KRAS inhibitor. In some embodiments, the kit comprises a pharmaceutical composition comprising cetuximab and a pharmaceutically acceptable carrier, excipient, binder, or diluent. In some embodiments, the kit comprises a pharmaceutical composition comprising trametinib or a salt thereof and a pharmaceutically acceptable carrier, excipient, binder, or diluent. In some embodiments, the kit comprises a pharmaceutical composition comprising palbociclib or a salt thereof and a pharmaceutically acceptable carrier, excipient, binder, or diluent. In some embodiments, cetuximab is formulated for administration to a subject via intravenous infusion. In some embodiments, trametinib or a salt thereof is formulated for oral administration to a subject. In some embodiments, palbociclib or a salt thereof is formulated for oral administration to a subject. In some embodiments, cetuximab, trametinib or a salt thereof, and palbociclib or a salt thereof are formulated in different compositions. In some embodiments, trametinib or a salt thereof, and palbociclib or a salt thereof are formulated in one composition.

In some embodiments, cetuximab, trametinib or a salt thereof, and palbociclib or a salt thereof are formulated as individual compositions. In some embodiments, cetuximab and palbociclib or a salt thereof are formulated as one composition. In some embodiments, trametinib or a salt thereof and palbociclib or a salt thereof are formulated as one composition. In some embodiments, cetuximab, trametinib or a salt thereof, and palbociclib or a salt thereof are formulated in liquid forms. In some embodiments, cetuximab, trametinib or a salt thereof, and palbociclib or a salt thereof are formulated in solid forms.

In some embodiments, cetuximab, trametinib or a salt thereof, and palbociclib or a salt thereof are administered separately. In some embodiments, cetuximab, trametinib or a salt thereof, and palbociclib or a salt thereof are administered simultaneously. In some embodiments, cetuximab, trametinib or a salt thereof, and palbociclib or a salt thereof are administered continuously. In some embodiments, cetuximab, trametinib or a salt thereof, and palbociclib or a salt thereof are administered intermittently. In some embodiments, cetuximab, trametinib or a salt thereof, and palbociclib or a salt thereof are administered with different dosing frequencies.

In some embodiments, the kit comprises osimertinib or a salt thereof, trametinib or a salt thereof, and palbociclib or a salt thereof. In some embodiments, the kit does not comprise a KRAS inhibitor. In some embodiments, the kit comprises a pharmaceutical composition comprising osimertinib or a salt thereof and a pharmaceutically acceptable carrier, excipient, binder, or diluent. In some embodiments, the kit comprises a pharmaceutical composition comprising trametinib or a salt thereof and a pharmaceutically acceptable carrier, excipient, binder, or diluent. In some embodiments, the kit comprises a pharmaceutical composition comprising palbociclib or a salt thereof and a pharmaceutically acceptable carrier, excipient, binder, or diluent. In some embodiments, the osimertinib or a salt thereof is formulated for oral administration to a subject. In some embodiments, the trametinib or a salt thereof is formulated for oral administration to a subject. In some embodiments, the palbociclib or a salt thereof is formulated for oral administration to a subject.

In some embodiments, osimertinib or a salt thereof, trametinib or a salt thereof, and palbociclib or a salt thereof are formulated as one composition. In some embodiments, osimertinib or a salt thereof, trametinib or a salt thereof, and palbociclib or a salt thereof are formulated as individual compositions. In some embodiments, osimertinib or a salt thereof and trametinib or a salt thereof are formulated as one composition. In some embodiments, osimertinib or a salt thereof and palbociclib or a salt thereof are formulated as one composition. In some embodiments, trametinib or a salt thereof and palbociclib or a salt thereof are formulated as one composition. In some embodiments, osimertinib or a salt thereof, trametinib or a salt thereof, and palbociclib or a salt thereof are formulated in liquid forms. In some embodiments, osimertinib or a salt thereof, trametinib or a salt thereof, and palbociclib or a salt thereof are formulated in solid forms.

In some embodiments, osimertinib or a salt thereof, trametinib or a salt thereof, and palbociclib or a salt thereof are administered as one composition. In some embodiments, osimertinib or a salt thereof, trametinib or a salt thereof, and palbociclib or a salt thereof are administered separately. In some embodiments, osimertinib or a salt thereof, trametinib or a salt thereof, and palbociclib or a salt thereof are administered simultaneously. In some embodiments, osimertinib or a salt thereof, trametinib or a salt thereof, and palbociclib or a salt thereof are administered continuously. In some embodiments, osimertinib or a salt thereof, trametinib or a salt thereof, and palbociclib or a salt thereof are administered intermittently.

In some embodiments, the kit comprises cetuximab, cobimetinib or a salt thereof, and abemaciclib or a salt thereof. In some embodiments, the kit does not comprise a KRAS inhibitor. In some embodiments, the kit comprises a pharmaceutical composition comprising cetuximab and a pharmaceutically acceptable carrier, excipient, binder, or diluent. In some embodiments, the kit comprises a pharmaceutical composition comprising cobimetinib or a salt thereof and a pharmaceutically acceptable carrier, excipient, binder, or diluent. In some embodiments, the kit comprises a pharmaceutical composition comprising abemaciclib or a salt thereof and a pharmaceutically acceptable carrier, excipient, binder, or diluent. In some embodiments, cetuximab is formulated for administration to a subject via intravenous infusion. In some embodiments, cobimetinib or a salt thereof is formulated for oral administration to a subject. In some embodiments, abemaciclib or a salt thereof is formulated for oral administration to a subject. In some embodiments, cetuximab, cobimetinib or a salt thereof, and abemaciclib or a salt thereof are formulated in different compositions. In some embodiments, cobimetinib or a salt thereof, and abemaciclib or a salt thereof are formulated in one composition.

In some embodiments, cetuximab, cobimetinib or a salt thereof, and abemaciclib or a salt thereof are formulated as one composition. In some embodiments, cetuximab, cobimetinib or a salt thereof, and abemaciclib or a salt thereof are formulated as individual compositions. In some embodiments, cetuximab and abemaciclib or a salt thereof are formulated as one composition. In some embodiments, cobimetinib or a salt thereof and abemaciclib or a salt thereof are formulated as one composition. In some embodiments, cetuximab, cobimetinib or a salt thereof, and abemaciclib or a salt thereof are formulated in liquid forms. In some embodiments, cetuximab, cobimetinib or a salt thereof, and abemaciclib or a salt thereof are formulated in solid forms.

In some embodiments, cetuximab, cobimetinib or a salt thereof, and abemaciclib or a salt thereof are administered separately. In some embodiments, cetuximab, cobimetinib or a salt thereof, and abemaciclib or a salt thereof are administered simultaneously. In some embodiments, cetuximab, cobimetinib or a salt thereof, and abemaciclib or a salt thereof are administered continuously. In some embodiments, cetuximab, cobimetinib or a salt thereof, and abemaciclib or a salt thereof are administered intermittently. In some embodiments, cetuximab, cobimetinib or a salt thereof, and abemaciclib or a salt thereof are administered with different dosing frequencies.

In some embodiments, the kit comprises lapatinib or a salt thereof, trametinib or a salt thereof, and palbociclib or a salt thereof. In some embodiments, the kit does not comprise a KRAS inhibitor. In some embodiments, the kit comprises a pharmaceutical composition comprising lapatinib or a salt thereof and a pharmaceutically acceptable carrier, excipient, binder, or diluent. In some embodiments, the kit comprises a pharmaceutical composition comprising trametinib or a salt thereof and a pharmaceutically acceptable carrier, excipient, binder, or diluent. In some embodiments, the kit comprises a pharmaceutical composition comprising palbociclib or a salt thereof and a pharmaceutically acceptable carrier, excipient, binder, or diluent. In some embodiments, lapatinib or a salt thereof is formulated for oral administration to a subject. In some embodiments, trametinib or a salt thereof is formulated for oral administration to a subject. In some embodiments, palbociclib or a salt thereof is formulated for oral administration to a subject. In some embodiments, lapatinib or a salt thereof, trametinib or a salt thereof, and palbociclib or a salt thereof are formulated in different compositions. In some embodiments, lapatinib or a salt thereof, trametinib or a salt thereof, and palbociclib or a salt thereof are formulated in one composition.

In some embodiments, lapatinib or a salt thereof, trametinib or a salt thereof, and palbociclib or a salt thereof are formulated as one composition. In some embodiments, lapatinib or a salt thereof, trametinib or a salt thereof, and palbociclib or a salt thereof are formulated as individual compositions. In some embodiments, lapatinib or a salt thereof and palbociclib or a salt thereof are formulated as one composition. In some embodiments, trametinib or a salt thereof and palbociclib or a salt thereof are formulated as one composition. In some embodiments, lapatinib or a salt thereof, trametinib or a salt thereof, and palbociclib or a salt thereof are formulated in liquid forms. In some embodiments, lapatinib or a salt thereof, trametinib or a salt thereof, and palbociclib or a salt thereof are formulated in solid forms.

In some embodiments, lapatinib or a salt thereof, trametinib or a salt thereof, and palbociclib or a salt thereof are administered as one composition. In some embodiments, lapatinib or a salt thereof, trametinib or a salt thereof, and palbociclib or a salt thereof are administered separately. In some embodiments, lapatinib or a salt thereof, trametinib or a salt thereof, and palbociclib or a salt thereof are administered simultaneously. In some embodiments, lapatinib or a salt thereof, trametinib or a salt thereof, and palbociclib or a salt thereof are administered continuously. In some embodiments, lapatinib or a salt thereof, trametinib or a salt thereof, and palbociclib or a salt thereof are administered intermittently. In some embodiments, lapatinib or a salt thereof, trametinib or a salt thereof, and palbociclib or a salt thereof are administered with different dosing frequencies.

In some embodiments, the kit comprises a package insert containing instructions regarding indications, usage, dosage, administration, contraindications, other medicaments to be combined with the packaged product, and/or warnings concerning the use of such medicaments.

### BRIEF DESCRIPTION OF THE DRAWINGS

FIGS. 1A and 1B depict tumor volume reduction in mice upon treatment with combination therapy in a KRAS^{G12D} CRC PDX Model with patient CKY041-P1. FIG. 1A shows the mean tumor volume changes while FIG. 1B shows the ratio of tumor volume of treated versus control mice over time during the combination treatment: (empty square)—control with no treatment; and (solid circle)—lapatinib 100 mg/kg, trametinib 0.3 mg/kg, and palbociclib 75 mg/kg daily (p.o.) for 21 days. The dosage unit, mg/kg, refers to dose of the compound per kg of the mouse body weight.

FIGS. 2A and 2B depict tumor volume reduction in mice upon treatment with combination therapy in a KRAS^{G12D} CRC PDX Model with patient NYL170-P2. FIG. 2A shows the mean tumor volume changes while FIG. 2B shows the ratio of tumor volume of treated versus control mice over time during the combination treatment: (empty square)—control with no treatment; (solid circle)—cetuximab 1 mg/animal weekly (i.p.) for 3 weeks, trametinib 0.3 mg/kg and palbociclib 75 mg/kg daily (p.o.) for 21 days; (solid diamond)—cetuximab 1 mg/animal weekly (i.p.) for 3 weeks; (solid square)—trametinib 0.3 mg/kg daily (p.o.) for 21 days; and (empty triangle)—palbociclib 75 mg/kg daily (p.o.) for 21 days. The dosage unit, mg/kg, refers to dose of the compound per kg of the mouse body weight.

FIGS. 3A and 3B depict tumor volume reduction in mice upon treatment with combination therapy in a KRAS^{G12D} CRC PDX Model with patient NYL170-P3. FIG. 3A shows the mean tumor volume changes while FIG. 3B shows the ratio of tumor volume of treated versus control mice over time during the combination treatment: (empty square)—control with no treatment; (solid circle)—lapatinib 100 mg/kg, trametinib 0.3 mg/kg, and palbociclib 75 mg/kg daily (p.o.) for 21 days; and (solid diamond)—lapatinib 50 mg/kg, trametinib 0.15 mg/kg, and palbociclib 37.5 mg/kg daily (p.o.) for 21 days. The dosage unit, mg/kg, refers to dose of the compound per kg of the mouse body weight.

FIGS. 4A and 4B depict tumor volume reduction in mice upon treatment with combination therapy in a KRAS^{G13D} CRC PDX Model with patient NYL178-P2. FIG. 4A shows the mean tumor volume changes while FIG. 4B shows the ratio of tumor volume of treated versus control mice over time during the combination treatment: (empty square)—control with no treatment; (empty triangle)—cetuximab 1 mg/animal weekly (i.p.) for 3 weeks, trametinib 0.3 mg/kg and palbociclib 75 mg/kg daily (p.o.) for 21 days; (patterned square)—lapatinib 100 mg/kg, trametinib 0.3 mg/kg, and palbociclib 75 mg/kg daily (p.o.) for 21 days; (solid circle)—cetuximab 1 mg/animal weekly (i.p.) for 3 weeks and palbociclib 75 mg/kg daily (p.o.) for 21 days; (solid triangle)—cetuximab 1 mg/animal weekly (i.p.) for 3 weeks and trametinib 0.3 mg/kg daily (p.o.) for 21 days; (empty diamond)—trametinib 0.3 mg/kg and palbociclib 75 mg/kg daily (p.o.) for 21 days; (solid diamond)—cetuximab 1 mg/animal weekly (i.p.) for 3 weeks; (empty circle)—trametinib 0.3 mg/kg daily (p.o.) for 21 days; and (solid square)—palbociclib 75 mg/kg daily (p.o.) for 21 days. The dosage unit, mg/kg, refers to dose of the compound per kg of the mouse body weight.

FIGS. 5A and 5B depict tumor volume reduction in mice upon treatment with combination therapy in a KRAS^{G13D} CRC PDX Model with patient NYL178-P4. FIG. 5A shows the mean tumor volume changes while FIG. 5B shows the ratio of tumor volume of treated versus control mice over time during the combination treatment: (empty square)—control with no treatment; (solid circle)—lapatinib 100 mg/kg daily (p.o.) for 14 days, cetuximab 1 mg/animal weekly (i.p.) for 2 weeks, and palbociclib 75 mg/kg daily (p.o.) for 14 days; and (solid diamond)—lapatinib 100 mg/kg, trametinib 0.3 mg/kg, and palbociclib 75 mg/kg daily (p.o.) for 14 days. The dosage unit, mg/kg, refers to dose of the compound per kg of the mouse body weight.

FIGS. 6A and 6B depict tumor volume reduction in mice upon treatment with combination therapy in a KRAS^{G12D} CRC PDX Model with patient NYL-JN-025. FIG. 6A shows the mean tumor volume changes while FIG. 6B shows the ratio of tumor volume of treated versus control mice over time during the combination treatment: (empty square)—control with no treatment; (solid circle)—cetuximab 1 mg/animal weekly (i.p.) for 3 weeks, trametinib 0.3 mg/kg daily (p.o.) for 21 days, and palbociclib 75 mg/kg daily (p.o.) for 21 days; (empty diamond)—cetuximab 1 mg/animal weekly (i.p.) for 3 weeks, and palbociclib 75 mg/kg daily (p.o.) for 21 days; (solid square)—cetuximab 1 mg/animal weekly (i.p.) for 3 weeks and trametinib 0.3 mg/kg (p.o.) for 21 days; (solid triangle)—trametinib 0.3 mg/kg daily (p.o.) for 21 days, and palbociclib 75 mg/kg daily (p.o.) for 21 days; (empty triangle)—cetuximab 1 mg/animal weekly (i.p.) for 3 weeks; (empty circle)—trametinib 0.3 mg/kg daily (p.o.) for 21 days; and (solid diamond)—palbociclib 75 mg/kg daily (p.o.) for 21 days. The dosage unit, mg/kg, refers to dose of the compound per kg of the mouse body weight.

FIGS. 7A and 7B depict tumor volume reduction in mice upon treatment with combination therapy in a KRAS^{G12V} CRC PDX Model with patient NYP031-P9. FIG. 7A shows the mean tumor volume changes while FIG. 7B shows the ratio of tumor volume of treated versus control mice over time during the combination treatment: (empty square)—control with no treatment; (solid circle)—cobimetinib, 5.2 mg/kg, AZD92910 10 mg/kg daily, and palbociclib 75 mg/kg daily (p.o.) for 21 days; (empty diamond)—cobimetinib, 2.6 mg/kg, AZD9291 10 mg/kg, and palbociclib 75 mg/kg daily (p.o.) for 21 days; and (solid triangle)—cobimetinib, 1.3 mg/kg, AZD9291 10 mg/kg, and palbociclib 75 mg/kg daily (p.o.) for 21 days. The dosage unit, mg/kg, refers to dose of the compound per kg of the mouse body weight.

FIGS. 8A and 8B depict tumor volume reduction in mice upon treatment with combination therapy in a KRAS^{G12D} CRC PDX Model with patient ZKB171-P2. FIG. 8A shows the mean tumor volume changes while FIG. 8B shows the ratio of tumor volume of treated versus control mice over time during the combination treatment: (empty square)—control with no treatment; (solid circle)—cetuximab 1 mg/animal weekly (i.p.) for 3 weeks, trametinib 0.3 mg/kg daily (p.o.) for 21 days, and palbociclib 75 mg/kg daily (p.o.) for 21 days; (empty circle)—cetuximab 1 mg/animal weekly (i.p.) for 3 weeks, and palbociclib 75 mg/kg daily (p.o.) for 21 days; (solid square)—cetuximab 1 mg/animal weekly (i.p.) for 3 weeks and trametinib 0.3 mg/kg (p.o.) for 21 days; (empty triangle)—trametinib 0.3 mg/kg daily (p.o.) for 21 days, and palbociclib 75 mg/kg daily (p.o.) for 21 days; (solid diamond)—cetuximab 1 mg/animal weekly (i.p.) for 3 weeks; (empty diamond)—trametinib 0.3 mg/kg daily (p.o.) for 21 days; and (solid triangle)—palbociclib 75 mg/kg daily (p.o.) for 21 days. The dosage unit, mg/kg, refers to dose of the compound per kg of the mouse body weight.

FIGS. 9A and 9B depict tumor volume reduction in mice upon treatment with combination therapy in a KRAS^{G12D} CRC PDX Model with patient ZKB182-P2. FIG. 9A shows the mean tumor volume changes while FIG. 9B shows the ratio of tumor volume of treated versus control mice over time during the combination treatment: (empty square)—control with no treatment; (solid circle)—lapatinib 100 mg/kg, trametinib 0.3 mg/kg, and palbociclib 75 mg/kg daily (p.o.) for 21 days; (solid diamond)—lapatinib 100 mg/kg daily (p.o.) for 21 days; (solid square)—trametinib 0.3 mg/kg (p.o.) for 21 days; (empty triangle)—palbociclib 75 mg/kg daily (p.o.) for 21 days. The dosage unit, mg/kg, refers to dose of the compound per kg of the mouse body weight.

FIGS. 10A and 10B depict in vivo studies with KRAS mutant colorectal cancer xenograft model HCT-116 (G13D). FIG. 10A shows combined inhibition of EGFR, MEK and CDK4/6 blocked HCT-116 tumor growth. Combinations of EGFR, BRAF and MEK inhibitors were less effective. FIG. 10B shows body weight change of the animals in FIG. 10A.

FIGS. 11A-11D show flow cytometry analysis for cellular apoptosis in NCI-H747 cells. FIGS. 11A and 11B were annexin V assays. FIG. 11A: vehicle control, FIG. 11B: cetuximab+cobimetinib+palbociclib treatment. FIGS. 11C and 11D were caspase 3 activity assays. FIG. 11C: vehicle control, FIG. 11D: cetuximab+cobimetinib+palbociclib treatment.

FIGS. 12A-12D show flow cytometry analysis for cellular apoptosis in NCT-116 cells. FIGS. 12A and 12B: annexin V assay. FIG. 12A: vehicle control, FIG. 12B: cetuximab+cobimetinib+palbociclib treatment. FIGS. 12C and 12D: caspase 3 activity assay, FIG. 12C: vehicle control, FIG. 12D: cetuximab+cobimetinib+palbociclib treatment.

FIGS. 13A and 13B show Annexin V assay for cellular apoptosis in SW-480 cells. FIG. 13A: vehicle control; FIG. 13B: cetuximab+cobimetinib+palbociclib treatment.

FIGS. 14A and 14B show Annexin V assay for cellular apoptosis in LS-180 cells. FIG. 13A: vehicle control; FIG. 13B: cetuximab+cobimetinib+palbociclib treatment.

FIG. 15 shows β-galacsidase activity assay in HCT-116 cells.

FIG. 16 shows β-galacsidase activity assay in SW480 cells.

FIG. 17 shows β-galacsidase activity assay in LS-180 cells.

### DETAILED DESCRIPTION

The present description is based on the inventor's data showing that a combination of an epidermal growth factor receptor inhibitor (such as osimertinib and cetuximab), a mitogen-activated protein kinase 1/2 inhibitor (such as cobimetinib) and a cyclin dependent kinase 4/6 inhibitor (such as palbociclib or abemaciclib) provides a robust therapy for a method of treating or delaying progression of cancer with a KRAS mutation (e.g., colorectal cancer). Particularly, such a combination therapy does not require a KRAS inhibitor. Without being bound by any theory or hypothesis, one of the advantages of the combination therapy described herein compared to a KRAS inhibitor is that it may be used for any type of KRAS mutation. Current KRAS inhibitors mainly focus on inhibitions of KRAS G12C mutation which only account for a subset of KRAS mutations in cancer; however, the combination therapy described herein has shown remarkable efficacy toward most known KRAS mutations. The combination therapy described herein has surprisingly demonstrated a synergistic effect toward cancers with a KRAS mutation and a robust efficacy in inhibiting the tumor growth by up to 95% in well-established animal models, despite the fact that none of the compounds in the combination is an inhibitor of the mutant KRAS. The description also provides compositions and kits that can be used for carrying out this combination therapy.

### 1. Definitions

Unless defined otherwise, all technical and scientific terms used herein have the same meaning as is commonly understood by one of ordinary skill in the art to which this disclosure belongs. All patents, applications, published applications and other publications referred to herein are incorporated by reference in their entireties. If a definition set forth in this section is contrary to or otherwise inconsistent with a definition set forth in a patent, application, or other publication that is herein incorporated by reference, the definition set forth in this section prevails over the definition incorporated herein by reference.

It is appreciated that certain features of the disclosure, which are, for clarity, described in the context of separate embodiments, may also be provided in combination in a single embodiment. Conversely, various features of the disclosure, which are, for brevity, described in the context of a single embodiment, may also be provided separately or in any suitable sub-combination. All combinations of the embodiments pertaining to particular method steps, reagents, or conditions are specifically embraced by the present disclosure and are disclosed herein just as if each and every combination was individually and explicitly disclosed.

As used herein and in the appended claims, the singular forms “a,” “an,” and “the” include plural referents unless the context clearly dictates otherwise. It is further noted that the claims may be drafted to exclude any optional element. As such, this statement is intended to serve as antecedent basis for use of such exclusive terminology as “solely,” “only” and the like in connection with the recitation of claim elements, or use of a “negative” limitation.

As used herein, the terms “including,” “containing,” and “comprising” are used in their open, non-limiting sense. It is also understood that aspects and embodiments of the invention described herein may include “consisting” and/or “consisting essentially of” aspects and embodiments.

It is understood that, whether the term “about” is used explicitly or not, every quantity given herein is meant to refer to the actual given value, and it is also meant to refer to the approximation to such given value that would reasonably be inferred based on the ordinary skill in the art, including equivalents and approximations due to the experimental and/or measurement conditions for such given value.

As used herein, a subject “at risk” of developing a disease may or may not have detectable disease, or symptoms of disease, and may or may not have displayed detectable disease or symptoms of disease prior to the treatment methods described herein. A subject “at risk” has one or more risk factors, which are measurable parameters that correlate with development of a disease (such as cancer), as described herein and known in the art. A subject “at risk” may have one or more risk factors. A subject having one or more risk factors has higher probability of developing the disease than a subject without one or more risk factors.

As used herein, “cancer” and “cancerous” refer to or describe the physiological condition in mammals that is typically characterized by unregulated cell growth. Examples of cancer include but are not limited to, carcinoma, lymphoma, blastoma, sarcoma, and leukemia. More particular examples of such cancers include but are not limited to squamous cell cancer, lung cancer (including small-cell lung cancer, non-small cell lung cancer, adenocarcinoma of the lung, and squamous carcinoma of the lung), cancer of the peritoneum, hepatocellular cancer, gastric or stomach cancer (including gastrointestinal cancer), pancreatic cancer, glioblastoma, cervical cancer, ovarian cancer, liver cancer, bladder cancer, hepatoma, breast cancer, colon cancer, colorectal cancer, endometrial or uterine carcinoma, salivary gland carcinoma, kidney or renal cancer, liver cancer, prostate cancer, vulval cancer, thyroid cancer, hepatic carcinoma and various types of head and neck cancer, as well as B-cell lymphoma (including low grade/follicular non-Hodgkin's lymphoma (NHL); small lymphocytic (SL) NHL; intermediate grade/follicular NHL; intermediate grade diffuse NHL; high grade immunoblastic NHL; high grade lymphoblastic NHL; high grade small non-cleaved cell NHL; bulky disease NHL; mantle cell lymphoma; AIDS-related lymphoma; and Waldenstrom's Macroglobulinemia); chronic lymphocytic leukemia (CLL); acute lymphoblastic leukemia (ALL); Hairy cell leukemia; chronic myeloblastic leukemia; and post-transplant lymphoproliferative disorder (PTLD), as well as abnormal vascular proliferation associated with phakomatoses, edema (such as that associated with brain tumors), and Meigs' syndrome. Examples of cancer may include primary tumors of any of the above types of cancer or metastatic tumors at a second site derived from any of the above types of cancer. Included in this definition are benign and malignant cancers as well as dormant tumors or micrometastases.

The terms “neoplastic cell”, “tumor cell”, or “cancer cell”, used either in the singular or plural form, refer to cells that have undergone a malignant transformation that makes them pathological to the host organism. Primary cancer cells (that is, cells obtained from near the site of malignant transformation) can be readily distinguished from non-cancerous cells by well-established techniques, particularly histological examination. The definition of a cancer cell, as used herein, includes not only a primary cancer cell, but any cell derived from a cancer cell ancestor. This includes metastasized cancer cells, and in vitro cultures and cell lines derived from cancer cells. When referring to a type of cancer that normally manifests as a solid tumor, a “clinically detectable” tumor is one that is detectable on the basis of tumor mass; e.g., by such procedures as CAT scan, magnetic resonance imaging (MRI), X-ray, ultrasound or palpation. Biochemical or immunologic findings alone may be insufficient to meet this definition.

As used herein, a “carrier” includes pharmaceutically acceptable carriers, excipients, or stabilizers that are nontoxic to the cell or mammal being exposed thereto at the dosages and concentrations employed. Often the physiologically acceptable carrier is an aqueous pH buffered solution. Non-limiting examples of physiologically acceptable carriers include buffers such as phosphate, citrate, and other organic acids; antioxidants including ascorbic acid; low molecular weight (less than about 10 residues) polypeptide; proteins, such as serum albumin, gelatin, or immunoglobulins; hydrophilic polymers such as polyvinylpyrrolidone; amino acids such as glycine, glutamine, asparagine, arginine or lysine; monosaccharides, disaccharides, and other carbohydrates including glucose, mannose, or dextrins; chelating agents such as EDTA; sugar alcohols such as mannitol or sorbitol; salt-forming counterions such as sodium; and/or nonionic surfactants such as TWEEN™, polyethylene glycol (PEG), and PLURONICS™.

As used herein, “delaying progression” of a disease means to defer, hinder, slow, retard, stabilize, and/or postpone development of the disease (such as cancer). This delay can be of varying lengths of time, depending on the history of the disease and/or individual being treated. As is evident to one skilled in the art, a sufficient or significant delay can, in effect, encompass prevention, in that the individual does not develop the disease. For example, a late stage cancer, such as development of metastasis, may be delayed. A method that “delays” development of cancer is a method that reduces probability of disease development in a given time frame and/or reduces the extent of the disease in a given time frame, when compared to not using the method. Such comparisons are typically based on clinical studies, using a statistically significant number of subjects. Cancer development can be detectable using standard methods, including, but not limited to, computerized axial tomography (CAT scan), Magnetic Resonance Imaging (MRI), ultrasound, clotting tests, arteriography, biopsy, urine cytology, and cystoscopy. Development may also refer to cancer progression that may be initially undetectable and includes occurrence, recurrence, and onset.

As used herein, the term “effective amount” or “therapeutically effective amount” of a substance is at least the minimum concentration required to effect a measurable improvement or prevention of a particular disorder. An effective amount herein may vary according to factors such as the disease state, age, sex, and weight of the patient, and the ability of the substance to elicit a desired response in the individual. An effective amount is also one in which any toxic or detrimental effects of the treatment are outweighed by the therapeutically beneficial effects. In reference to cancer, an effective amount comprises an amount sufficient to cause a tumor to shrink and/or to decrease the growth rate of the tumor (such as to suppress tumor growth) or to prevent or delay other unwanted cell proliferation in cancer. In some embodiments, an effective amount is an amount sufficient to delay development of cancer. In some embodiments, an effective amount is an amount sufficient to prevent or delay recurrence. In some embodiments, an effective amount is an amount sufficient to reduce recurrence rate in the individual. An effective amount can be administered in one or more administrations. The effective amount of the drug or composition may: (i) reduce the number of cancer cells; (ii) reduce tumor size; (iii) inhibit, retard, slow to some extent and preferably stop cancer cell infiltration into peripheral organs; (iv) inhibit (i.e., slow to some extent and preferably stop) tumor metastasis; (v) inhibit tumor growth; (vi) prevent or delay occurrence and/or recurrence of tumor; (vii) reduce recurrence rate of tumor, and/or (viii) relieve to some extent one or more of the symptoms associated with the cancer. An effective amount can be administered in one or more administrations. For purposes of this disclosure, an effective amount of drug, compound, or pharmaceutical composition is an amount sufficient to accomplish prophylactic or therapeutic treatment either directly or indirectly. As is understood in the clinical context, an effective amount of a drug, compound, or pharmaceutical composition may or may not be achieved in conjunction with another drug, compound, or pharmaceutical composition. Thus, an “effective amount” may be considered in the context of administering one or more therapeutic agents, and a single agent may be considered to be given in an effective amount if, in conjunction with one or more other agents, a desirable result may be or is achieved.

As used herein, the term “inhibitor” or “antagonist” refers to biological or chemical substance that interferes with or otherwise reduces the physiological and/or biochemical action of another biological or chemical molecule. In some embodiments, the inhibitor or antagonist specifically binds to the other molecule.

A “package insert” refers to instructions customarily included in commercial packages of medicaments that contain information about the indications customarily included in commercial packages of medicaments that contain information about the indications, usage, dosage, administration, contraindications, other medicaments to be combined with the packaged product, and/or warnings concerning the use of such medicaments, etc.

A “pharmaceutically acceptable salt” is a salt form that is non-toxic, biologically tolerable, or otherwise biologically suitable for administration to the subject. See generally Berge et al. (1977) J. Pharm. Sci. 66, 1. Particular pharmaceutically acceptable salts are those that are pharmacologically effective and suitable for contact with the tissues of subjects without undue toxicity, irritation, or allergic response. Pharmaceutically acceptable salts include, without limitation, acid addition salts, formed with inorganic acids such as hydrochloric acid, hydrobromic acid, sulfuric acid, nitric acid, phosphoric acid, and the like; or formed with organic acids such as acetic acid, oxalic acid, propionic acid, succinic acid, maleic acid, tartaric acid and the like. These salts may be derived from inorganic or organic acids. Non-limiting examples of pharmaceutically acceptable salts include, without limitation, sulfates, pyrosulfates, bisulfates, sulfites, bisulfites, phosphates, monohydrogen-phosphates, dihydrogenphosphates, metaphosphates, pyrophosphates, chlorides, bromides, iodides, acetates, propionates, decanoates, caprylates, acrylates, formates, isobutyrates, caproates, heptanoates, propiolates, oxalates, malonates, succinates, suberates, sebacates, fumarates, maleates, butyne-1,4-dioates, hexyne-1,6-dioates, benzoates, chlorobenzoates, methylbenzoates, dinitrobenzoates, hydroxybenzoates, methoxybenzoates, phthalates, sulfonates, methylsulfonates, propylsulfonates, besylates, xylenesulfonates, naphthalene-1-sulfonates, naphthalene-2-sulfonates, phenylacetates, phenylpropionates, phenylbutyrates, citrates, lactates, γ-hydroxybutyrates, glycolates, tartrates, and mandelates. In some embodiments, pharmaceutically acceptable salts are formed when an acidic proton present in the parent compound either is replaced by a metal ion, e.g., an alkali metal ion, an alkaline earth ion, or an aluminum ion; or coordinates with an organic base. Salts derived from pharmaceutically acceptable organic non-toxic bases include, without limitation, salts of primary, secondary, and tertiary amines, substituted amines including naturally occurring substituted amines, cyclic amines and basic ion exchange resins, such as isopropylamine, trimethylamine, diethylamine, triethylamine, tripropylamine, ethanolamine, 2-diethylaminoethanol, tromethamine, trimetharnine, dicyclohexylamine, caffeine, procaine, hydrabamine, choline, betaine, ethylenediamine, glucosamine, N-ethylglucamine, N-methylglucamine, theobromine, purines, piperazine, piperidine, N-ethylpiperidine, polyamine resins, amino acids such as lysine, arginine, histidine, and the like. Examples of pharmaceutically acceptable base addition salts include those derived from inorganic bases such as sodium, potassium, lithium, ammonium, calcium, magnesium, iron, zinc, copper, manganese, aluminum salts and the like. In some embodiments, the organic non-toxic bases are L-amino acids, such as L-lysine and L-arginine, tromethamine, N-ethylglucamine and N-methylglucamine. Acceptable inorganic bases include, without limitation, aluminum hydroxide, calcium hydroxide, potassium hydroxide, sodium carbonate, sodium hydroxide, and the like. Lists of other suitable pharmaceutically acceptable salts are found in Remington's Pharmaceutical Sciences, 17th Edition, Mack Publishing Company, Easton, Pa., 1985.

A “solvate” is formed by the interaction of a solvent and a compound. Suitable solvents include, for example, water and alcohols (e.g., ethanol). Solvates include hydrates having any ratio of compound to water, such as monohydrates, dihydrates and hemi-hydrates.

A “subject,” “patient” or “individual” includes a mammal, such as a human or other animal, and typically is human. In some embodiments, the subject, e.g., patient, to whom the therapeutic agents and compositions are administered, is a mammal, typically a primate, such as a human. In some embodiments, the primate is a monkey or an ape. The subject can be male or female and can be any suitable age, including infant, juvenile, adolescent, adult, and geriatric subjects. In some embodiments, the subject is a non-primate mammal, such as a rodent, a dog, a cat, a farm animal, such as a cow or a horse, etc.

As used herein, a “tissue sample” or “cell sample” is meant a collection of similar cells obtained from a tissue of a subject or patient. The source of the tissue or cell sample may be solid tissue as from a fresh, frozen and/or preserved organ or tissue sample or biopsy or aspirate; blood or any blood constituents; bodily fluids such as cerebral spinal fluid, amniotic fluid, peritoneal fluid, or interstitial fluid; cells from any time in gestation or development of the subject. The tissue sample may also be primary or cultured cells or cell lines. Optionally, the tissue or cell sample is obtained from a disease tissue/organ, such as a cancer or tumor tissue. The tissue sample may contain compounds which are not naturally intermixed with the tissue in nature such as preservatives, anticoagulants, buffers, fixatives, nutrients, antibiotics, or the like.

As used herein, the term “treatment” refers to clinical intervention designed to have beneficial and desired effects to the natural course of the individual or cell being treated during the course of clinical pathology. For the purpose of this disclosure, desirable effects of treatment include, without limitation, decreasing the rate of disease progression, ameliorating or palliating the disease state, and remission or improved prognosis. For example, an individual is successfully “treated” if one or more symptoms associated with cancer are mitigated or eliminated, including, but are not limited to, reducing the proliferation of (or destroying) cancerous cells, increasing cancer cell-killing, decreasing symptoms resulting from the disease, preventing spread of diseases, preventing recurrence of disease, increasing the quality of life of those suffering from the disease, decreasing the dose of other medications required to treat the disease, delaying the progression of the disease, and/or prolonging survival of individuals.

### II. Methods of Treating and Delaying Progression of Cancer with KRAS Mutation

Provided herein are methods for treating or delaying progression of cancer (e.g., colorectal cancer) in a subject comprising administering to the subject an effective amount of (a) an epidermal growth factor receptor (EGFR) inhibitor; (b) a mitogen-activated protein kinase (MEK) 1/2 inhibitor; and (c) a cyclin dependent kinase (CDK) 4/6 inhibitor; wherein the subject has cancer (e.g., colorectal cancer) that has a KRAS mutation or is at risk of developing cancer (e.g., colorectal cancer) that has a KRAS mutation.

In some embodiments of the methods described herein, (a) is a small molecule or antibody (or antigen-binding fragment thereof) which specifically binds to EGFR or a ligand thereof, and is optionally selected from one or more of cetuximab, panitumumab, zalutumumab, nimotuzumab, matuzumab, erlotinib, gefitinib, afatinib, lapatinib, osimertinib, brigatinib, and icotinib (including salt forms of any of the compounds). In some embodiments, (b) is a small molecule or antibody (or antigen-binding fragment thereof) which specifically binds to a MEK 1/2 or a ligand thereof, and is optionally selected from one or more of trametinib, selumetinib, TAK-733, CI-1040, PD0325901, MEK162, AZD8330, GDC-0623, refametinib, pimasertib, R04987655, R05126766, WX-554, HL-085, binimetnib, and cobimetinib (including salt forms of any of the compounds). See Chen et al. (2017) Molecules 22, 1551. In some embodiments, (c) is a small molecule or antibody (or antigen-binding fragment thereof) which specifically binds to a CDK 4/6 or a ligand thereof, and is optionally selected from one or more of palbociclib, ribociclib, and abemaciclib (including salt forms of any of the compounds). In some embodiments, the method does not comprise administering a KRAS inhibitor to the subject during the administrations of (a) an epidermal growth factor receptor (EGFR) inhibitor; (b) a mitogen-activated protein kinase (MEK) 1/2 inhibitor; and (c) a cyclin dependent kinase (CDK) 4/6 inhibitor. In some embodiments, the method does not comprise administering an additional therapeutic agent during the administrations of (a) an epidermal growth factor receptor (EGFR) inhibitor; (b) a mitogen-activated protein kinase (MEK) 1/2 inhibitor; and (c) a cyclin dependent kinase (CDK) 4/6 inhibitor. In some embodiments, the subject had received a KRAS inhibitor during a previous treatment cycle. In some embodiments, the subject has not received a KRAS inhibitor during a previous treatment cycle. In some embodiments, the subject is a human.

In one aspect, the method disclosed here can be used to treat or delay progression of a cancer that has a KRAS mutation. KRAS is a GTPase and KRAS mutations have been found in various human cancers, including but not limited to, pancreatic carcinomas, colon carcinomas, lung carcinomas, biliary tract malignancies, endometrial cancer, cervical cancer, bladder cancer, liver cancer, myeloid leukemia and breast cancer. Oncogenic forms of the KRAS gene are particularly prevalent in pancreatic cancer, colorectal cancer and lung cancer. KRAS has been reported to be mutated at several sites, but the vast majority of mutations occur at the Gly residue of codon 12 and codon 13. Common mutations include G12C, G12D, G12V, G12A, G12S, G12R and G13D. See Jia et al. (2017) Oncol. Lett. 14, 6525. Detection of these mutations can be performed using conventional methods, such as the non-limiting example reported in Lasota et al. (2015) Am. J. Surg. Pathol. 38, 1235. In some embodiments, KRAS mutation is detected in tissue or cell samples containing cancer cells from a subject. In some embodiments, the KRAS mutation is a somatic mutation. In some embodiments, the method is used to treat or delay progression of a cancer that has a KRAS G12C mutation. In some embodiments, the method is used to treat or delay progression of a cancer that has a KRAS G12V mutation. In some embodiments, the method is used to treat or delay progression of a cancer that has a KRAS G12D mutation. In some embodiments, the method is used to treat or delay progression of a cancer that has a KRAS G13D mutation. In some embodiments, the method does not comprise administering a KRAS inhibitor to the subject during the administrations of (a) an epidermal growth factor receptor (EGFR) inhibitor; (b) a mitogen-activated protein kinase (MEK) 1/2 inhibitor; and (c) a cyclin dependent kinase (CDK) 4/6 inhibitor. In some embodiments, the method does not comprise administering an additional therapeutic agent during the administrations of (a) an epidermal growth factor receptor (EGFR) inhibitor; (b) a mitogen-activated protein kinase (MEK) 1/2 inhibitor; and (c) a cyclin dependent kinase (CDK) 4/6 inhibitor. In some embodiments, the subject had received a KRAS inhibitor during a previous treatment cycle. In some embodiments, the subject has not received a KRAS inhibitor during a previous treatment cycle.

In some embodiments, the cancer is an adenocarcinoma, a squamous cell carcinoma, an adenosquamous carcinoma, an anaplastic carcinoma, a large cell carcinoma, and a small cell carcinoma. In some embodiments, the cancer is melanoma and carcinoma, such as an epithelial neoplasm, a squamous cell neoplasm, a basal cell neoplasm, a transitional cell carcinoma, an adenocarcinoma, an adnexal or skin appendage neoplasm, a nucoepidermoid neoplasm, a cystic, mucinous, or Serous neoplasm, a ductal, lobular, or medullary neoplasm, an acinar cell neoplasm, and a complex epithelial neoplasm. In some embodiments, the carcinoma is a colon cancer, a gastric cancer, a lung cancer, a breast cancer, a pancreatic cancer, an oral cancer, a prostate cancer, a germline cancer, a rectal cancer, a liver cancer, a kidney cancer, and an ovarian cancer. In some embodiments, the cancer is a colorectal cancer. In some embodiments, the cancer is a late stage cancer, such as stage IV colorectal cancer. In some embodiments, the cancer is an advanced colorectal cancer.

Also provided here are methods for treating or delaying progression of cancer (e.g., colorectal cancer) in a subject comprising administering to the subject an effective amount of osimertinib or a salt thereof, cobimetinib or a salt thereof, and palbociclib or a salt thereof, wherein the subject has cancer (e.g., colorectal cancer) that has a KRAS mutation or is at risk of developing cancer (e.g., colorectal cancer) that has a KRAS mutation. In another aspect, provided here are methods for treating or delaying progression of cancer (e.g., colorectal cancer) in a subject comprising administering to the subject an effective amount of cetuximab, cobimetinib or a salt thereof, and palbociclib or a salt thereof, wherein the subject has cancer (e.g., colorectal cancer) that has a KRAS mutation or is at risk of developing cancer (e.g., colorectal cancer) that has a KRAS mutation. In another aspect, provided here are methods for treating or delaying progression of cancer (e.g., colorectal cancer) in a subject comprising administering to the subject an effective amount of cetuximab, TAK-733 or a salt thereof, and palbociclib or a salt thereof, wherein the subject has cancer (e.g., colorectal cancer) that has a KRAS mutation or is at risk of developing cancer (e.g., colorectal cancer) that has a KRAS mutation. In another aspect, provided here are methods for treating or delaying progression of cancer (e.g., colorectal cancer) in a subject comprising administering to the subject an effective amount of osimertinib or a salt thereof, TAK-733 or a salt thereof, and palbociclib or a salt thereof, wherein the subject has cancer (e.g., colorectal cancer) that has a KRAS mutation or is at risk of developing cancer (e.g., colorectal cancer) that has a KRAS mutation. In another aspect, provided here are methods for treating or delaying progression of cancer (e.g., colorectal cancer) in a subject comprising administering to the subject an effective amount of cetuximab, cobimetinib or a salt thereof, and abemaciclib or a salt thereof, wherein the subject has cancer (e.g., colorectal cancer) that has a KRAS mutation or is at risk of developing cancer (e.g., colorectal cancer) that has a KRAS mutation. In another aspect, provided here are methods for treating or delaying progression of cancer (e.g., colorectal cancer) in a subject comprising administering to the subject an effective amount of cetuximab, cobimetinib or a salt thereof, and abemaciclib or a salt thereof, wherein the subject has cancer (e.g., colorectal cancer) that has a KRAS mutation or is at risk of developing cancer (e.g., colorectal cancer) that has a KRAS mutation. In another aspect, provided here are methods for treating or delaying progression of cancer (e.g., colorectal cancer) in a subject comprising administering to the subject an effective amount of lapatinib, trametinib or a salt thereof, and palbociclib or a salt thereof, wherein the subject has cancer (e.g., colorectal cancer) that has a KRAS mutation or is at risk of developing cancer (e.g., colorectal cancer) that has a KRAS mutation. In another aspect, provided here are methods for treating or delaying progression of cancer (e.g., colorectal cancer) in a subject comprising administering to the subject an effective amount of cetuximab, binimetinib or a salt thereof, and palbociclib or a salt thereof, wherein the subject has cancer (e.g., colorectal cancer) that has a KRAS mutation or is at risk of developing cancer (e.g., colorectal cancer) that has a KRAS mutation. In another aspect, provided here are methods for treating or delaying progression of cancer (e.g., colorectal cancer) in a subject comprising administering to the subject an effective amount of osimertinib, binimetinib or a salt thereof, and palbociclib or a salt thereof, wherein the subject has cancer (e.g., colorectal cancer) that has a KRAS mutation or is at risk of developing cancer (e.g., colorectal cancer) that has a KRAS mutation.

Osimertinib is an oral, third-generation EGFR inhibitor approved for treating non-small cell lung cancer harboring EGFR mutations by U.S. FDA and European Commission (EC). Osimertinib targets EGFR tyrosine kinase inhibitor (TKI)-sensitizing mutations and particularly T790M that often contributes to acquired resistance to EGFR TKI therapy. Cobimetinib is a MEK inhibitor approved by U.S. FDA to be used in combination with vemurafenib, a BRAF inhibitor, for treating metastatic melanoma with BRAF V600E or V600K mutation. Cobimetinib and vemurafenib target different components of the MAPK/ERK pathway: MEK and BRAF respectively. Palbociclib was a CDK4/6 inhibitor approved by U.S. FDA for treating hormone receptor (HR) positive, human epidermal growth factor receptor 2 (HER2) negative advanced or metastatic breast cancer in combination with an aromatase inhibitor as initial endocrine based therapy in postmenopausal women. Cetuximab is a chimeric monoclonal antibody given by intravenous infusion and an EGFR inhibitor approved by U.S. FDA in 2009 for treatment of colon cancer with wild-type KRAS. TAK-733 is an orally bioavailable, non-ATP-competitive small-molecule MEK1/2 inhibitor that completed a Phase I clinical study. Lapatinib is a dual tyrosine kinase inhibitor which interrupts the HER2/neu and epidermal growth factor receptor (EGFR) pathways, approved by U.S. FDA in treating breast cancer. Abemaciclib is a U.S. FDA approved, orally available cyclin-dependent kinase (CDK) inhibitor that targets the CDK4 (cyclin D1) and CDK6 (cyclin D3) cell cycle pathway, with potential antineoplastic activity. The structures of osimertinib, TAK-733, cobimetinib, palbociclib, lapatinib, abemaciclib, and trametinib are shown below.

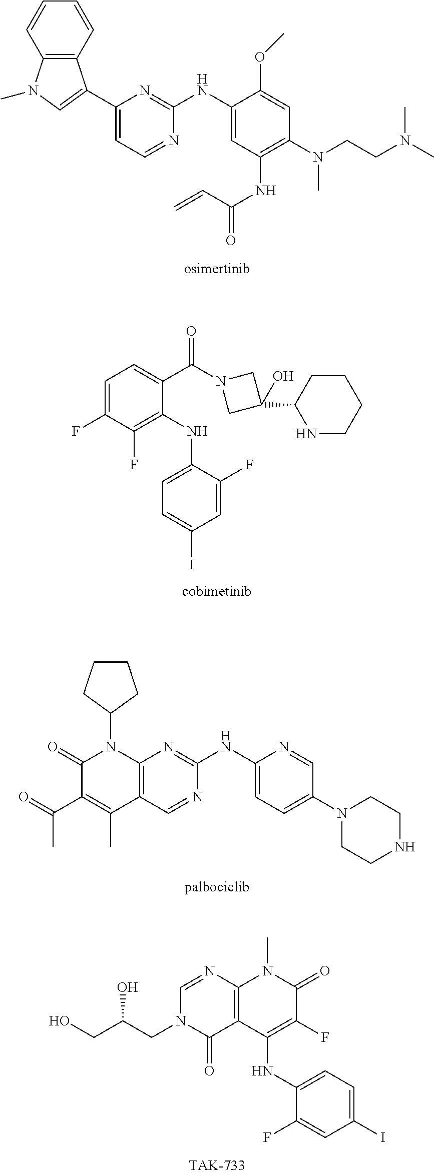

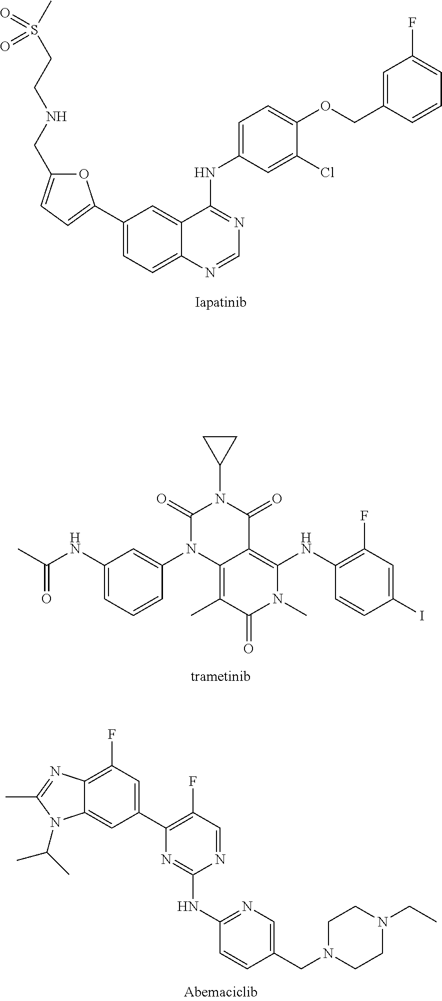 [SMILES: CS(=O)(=O)CCNCc1ccc(-c2ccc3ncnc(Nc4ccc(OCc5cccc(F)c5)c(Cl)c4)c3c2)o1 ; CC(=O)Nc1cccc(-n2c(=O)n(C3CC3)c(=O)c3c(Nc4ccc(I)cc4F)n(C)c(=O)c(C)c32)c1 ; CCN1CCN(Cc2ccc(Nc3ncc(F)c(-c4cc(F)c5nc(C)n(C(C)C)c5c4)n3)nc2)CC1]

In some embodiments, osimertinib, lapatinib, cobimetinib, trametinib, TAK-733, abemaciclib or palbociclib is administered alone or in combination in a salt form. In some embodiments, the salts are pharmaceutically acceptable salts. Non-limiting examples of pharmaceutically acceptable salts include, without limitation, sulfates, pyrosulfates, bisulfates, sulfites, bisulfites, phosphates, monohydrogen-phosphates, dihydrogenphosphates, metaphosphates, pyrophosphates, chlorides, bromides, iodides, acetates, propionates, decanoates, caprylates, acrylates, formates, isobutyrates, caproates, heptanoates, propiolates, oxalates, malonates, succinates, suberates, sebacates, fumarates, maleates, butyne-1,4-dioates, hexyne-1,6-dioates, benzoates, chlorobenzoates, methylbenzoates, dinitrobenzoates, hydroxybenzoates, methoxybenzoates, phthalates, sulfonates, methylsulfonates, mesylates propylsulfonates, besylates, xylenesulfonates, naphthalene-1-sulfonates, naphthalene-2-sulfonates, phenylacetates, phenylpropionates, phenylbutyrates, citrates, lactates, γ-hydroxybutyrates, glycolates, tartrates, and mandelates. In some embodiments, the pharmaceutically acceptable salts are fumarates. In some embodiments, the pharmaceutically acceptable salts are mesylates. In some embodiments, palbociclib is administered in a fumarate salt form. In some embodiments, cobimetinib is administered in a fumarate salt form. In some embodiments, palbociclib is administered in a hemifumarate salt form. In some embodiments, cobimetinib is administered in a hemifumarate salt form. In some embodiments, osimertinib or abemaciclib is administered in a mesylate salt form. In some embodiments, lapatinib is administered in the form of lapatinib ditosylate. In some embodiments, trametinib is administered in the form of trametinib dimethyl sulfoxide In some embodiments, osimertinib, cobimetinib, TAK-733 or palbociclib is administered in a solvate form.

The method may comprise administering any compositions or kits described herein.

In another aspect, the method comprises administering an effective amount of osimertinib or a salt thereof, cobimetinib or a salt thereof, and palbociclib or a salt thereof. In some embodiments, the daily dosage of osimertinib or a salt thereof is in the amount of about 0.25-0.5 mg/kg, about 0.5-1 mg/kg, about 1-1.5 mg/kg, about 1.5-2 mg/kg, about 2-2.5 mg/kg, about 2.5-3 mg/kg, about 3-3.5 mg/kg, about 3.5-4 mg/kg, or about 0.5-3 mg/kg. In some embodiments, the daily dosage of osimertinib or a salt thereof is in the amount of about 40 mg, about 80 mg, or about 160 mg. In some embodiments, the daily dosage of osimertinib or a salt thereof is in the amount of less than about 40 mg, about 80 mg, or about 160 mg. In some embodiments, the dosage of osimertinib or a salt thereof is in the amount of about 40-160 mg. In some embodiments, the daily dosage of osimertinib or a salt thereof is in the amount of about 0.6-2.7 mg/kg. In some embodiments, osimertinib is in its mesylate salt form. In some embodiments, osimertinib is in its mesylate salt form. The amounts of the inhibitor described herein and throughout the specification refer to the amount of the inhibitor without taking into consideration of the weight of the counterions if the inhibitor exists in a salt form. For example, the term “80 mg of osimertinib” could include, without limitation, 80 mg of osimertinib in a salt-free form or 95.4 mg of osimertinib mesylate.

In some embodiments, the daily dosage of cobimetinib or a salt thereof is in the amount of about 0.1-0.25 mg/kg, about 0.25-0.5 mg/kg, about 0.5-0.75 mg/kg, about 0.75-1 mg/kg, about 1-1.25 mg/kg, about 1.25-1.5 mg/kg, about 1.5-1.75 mg/kg, about 1.75-2 mg/kg, or about 0.25-1 mg/kg. In some embodiments, the daily dosage of cobimetinib or a salt thereof is in the amount of about 20 mg, about 40 mg, or about 60 mg. In some embodiments, the daily dosage of cobimetinib or a salt thereof is in the amount of less than about 20 mg, about 40 mg, or about 60 mg. In some embodiments, the daily dosage of cobimetinib or a salt thereof is in the amount of about 20-60 mg. In some embodiments, the daily dosage of cobimetinib or a salt thereof is in the amount of about 0.3-1 mg/kg. In some embodiments, the cobimetinib is in its hemifumarate salt form.

In some embodiments, the daily dosage of palbociclib or a salt thereof is in the amount of about 0.25-0.5 mg/kg, about 0.5-1 mg/kg, about 1.5-2 mg/kg, about 2-2.5 mg/kg, about 2.5-3 mg/kg, about 3-3.5 mg/kg, about 1-2.5 mg/kg, about 1-3 mg/kg, or about 3-5 mg/kg. In some embodiments, the daily dosage of palbociclib or a salt thereof is in the amount of about 15 mg, about 50 mg, about 75 mg, about 100 mg, or about 125 mg. In some embodiments, the daily dosage of palbociclib or a salt thereof is in the amount of less than about 15 mg, about 50 mg, about 75 mg, about 100 mg, or about 125 mg. In some embodiments, the daily dosage of palbociclib or a salt thereof is in the amount of about 15-125 mg. In some embodiments, the daily dosage of palbociclib or a salt thereof is in the amount of about 0.25-2.5 mg/kg. In some embodiments, the method comprises administration of osimertinib or a solvate or a salt thereof, cobimetinib or a solvate or a salt thereof, and palbociclib or a solvate or a salt thereof.

In some embodiments, the daily dosage of osimertinib or a salt thereof is in the amount that is about at greater than about 20%, or greater than about 25%, or greater than about 30%, or greater than about 35%, or greater than about 40%, or greater than about 45%, or greater than about 50%, or greater than about 55%, or greater than about 60%, or greater than about 65%, or greater than about 70%, or greater than about 75%, or greater than about 80%, or greater than about 85%, or greater than about 90%, or greater than 95% by weight of the combined daily dosage of osimertinib, cobimetinib, and palbociclib or salts of the foregoing. In some embodiments, the daily dosage of osimertinib or a salt thereof is in the amount that is about at less than 20%, or less than about 25%, or less than about 30%, or less than about 35%, or less than about 40%, or less than about 45%, or less than about 50%, or less than about 55%, or less than about 60%, or less than about 65%, or less than about 70%, or less than about 75%, or less than about 80%, or less than about 85%, or less than about 90%, or less than 95% by weight of the combined daily dosage of osimertinib, cobimetinib, and palbociclib or salts of the foregoing.

In some embodiments, the daily dosage of cobimetinib or a salt thereof is in the amount that is about at greater than about 20%, or greater than about 25%, or greater than about 30%, or greater than about 35%, or greater than about 40%, or greater than about 45%, or greater than about 50%, or greater than about 55%, or greater than about 60%, or greater than about 65%, or greater than about 70%, or greater than about 75%, or greater than about 80%, or greater than about 85%, or greater than about 90%, or greater than 95% by weight of the combined daily dosage of osimertinib, cobimetinib, and palbociclib or salts of the foregoing. In some embodiments, the daily dosage of cobimetinib or a salt thereof in the amount that is about at less than 20%, or less than about 25%, or less than about 30%, or less than about 35%, or less than about 40%, or less than about 45%, or less than about 50%, or less than about 55%, or less than about 60%, or less than about 65%, or less than about 70%, or less than about 75%, or less than about 80%, or less than about 85%, or less than about 90%, or less than 95% by weight of the combined daily dosage of osimertinib, cobimetinib, and palbociclib or salts of the foregoing.

In some embodiments, the daily dosage of palbociclib or a salt thereof is in the amount that is about at greater than about 20%, or greater than about 25%, or greater than about 30%, or greater than about 35%, or greater than about 40%, or greater than about 45%, or greater than about 50%, or greater than about 55%, or greater than about 60%, or greater than about 65%, or greater than about 70%, or greater than about 75%, or greater than about 80%, or greater than about 85%, or greater than about 90%, or greater than 95% by weight of the combined daily dosage of osimertinib, cobimetinib, and palbociclib or salts of the foregoing. In some embodiments, the daily dosage of palbociclib or a salt thereof is in the amount that is about at less than 20%, or less than about 25%, or less than about 30%, or less than about 35%, or less than about 40%, or less than about 45%, or less than about 50%, or less than about 55%, or less than about 60%, or less than about 65%, or less than about 70%, or less than about 75%, or less than about 80%, or less than about 85%, or less than about 90%, or less than 95% by weight of the combined daily dosage of osimertinib, cobimetinib, and palbociclib or salts of the foregoing.

In another aspect, the method comprises administering an effective amount of lapatinib or a salt thereof, trametinib or a salt thereof, and palbociclib or a salt thereof. In some embodiments, the daily dosage of lapatinib or a salt thereof is in the amount of about 2.5-5 mg/kg, about 5-10 mg/kg, about 10-15 mg/kg, about 15-20 mg/kg, about 20-25 mg/kg, about 25-30 mg/kg, about 30-35 mg/kg, about 35-40 mg/kg, or about 5-30 mg/kg. In some embodiments, the daily dosage of osimertinib or a salt thereof is in the amount of about 500 mg, about 1000 mg, about 1250 mg, about 1500 mg, about 4500 mg, or about 5500 mg. In some embodiments, the daily dosage of osimertinib or a salt thereof is in the amount of less than about 500 mg, about 1000 mg, about 1250 mg, about 1500 mg, about 4500 mg, or about 5500 mg. In some embodiments, the dosage of lapatinib or a salt thereof is in the amount of about 500-5500 mg. In some embodiments, the daily dosage of lapatinib or a salt thereof is in the amount of about 6-60 mg/kg. In some embodiments, lapatinib is in its ditosylate salt form. In some embodiments, lapatinib is in its ditosylate monohydrate salt form. The amounts of the inhibitor described herein and throughout the specification refer to the amount of the inhibitor without taking into consideration of the weight of the counterions if the inhibitor exists in a salt form. For example, the term “250 mg of lapatinib” could include, without limitation, 250 mg of lapatinib in a salt-free form or 398 mg of lapatinib ditosylate or 405 mg lapatinib ditosylate monohydrate.

In some embodiments, the daily dosage of trametinib or a salt thereof is in the amount of about 0.01-1 mg/kg, about 0.01-0.02 mg/kg, about 0.02-0.03 mg/kg, about 0.03-0.05 mg/kg, about 0.05-0.08 mg/kg, about 0.08-0.1 mg/kg, about 0.1-0.2 mg/kg, about 0.2-0.3 mg/kg, about 0.3-0.4 mg/kg, about 0.4-0.5 mg/kg, about 0.5-0.6 mg/kg, about 0.6-0.7 mg/kg, about 0.7-0.8 mg/kg, about 0.8-0.9 mg/kg, or about 0.9-1 mg/kg of trametinib. In some embodiments, the daily dosage of trametinib or a salt thereof is in the amount of about 0.5 mg, about 1 mg, about 2 mg, or about 4 mg of trametinib. In some embodiments, the daily dosage of trametinib or a salt thereof is in the amount of less than about 0.5 mg, less than about 1 mg, less than about 2 mg, or less than about 4 mg of trametinib. In some embodiments, the daily dosage of trametinib or a salt thereof is in the amount of about 0.1-25 mg, about 0.1-0.5 mg, about 0.5-1 mg, about 1-2 mg, about 2-4 mg, about 4-10 mg or about 0.5-2 mg of trametinib. In some embodiments, the daily dosage of trametinib of a salt thereof is 2 mg of trametinib. The amounts of the inhibitor described herein and throughout the specification refer to the amount of the inhibitor without taking into consideration of the weight of the counterions if the inhibitor exists in a salt form. For example, the term “0.5 mg of trametinib” could include, without limitation, 0.5 mg of trametinib in a salt-free form or 0.5635 mg of trametinib dimethyl sulfoxide.

In another aspect, the method comprises administering an effective amount of cetuximab, cobimetinib or a salt thereof, and abemaciclib or a salt thereof. In some embodiments, the dosage of abemaciclib is in the amount of about 0.25-0.5 mg/kg, about 0.5-1 mg/kg, about 1.5-2 mg/kg, about 2-2.5 mg/kg, about 2.5-3 mg/kg, about 3-3.5 mg/kg, about 1-2.5 mg/kg, about 1-3 mg/kg, about 3-5 mg/kg, about 4-5 mg/kg, about 4-6 mg/kg, about 3-6 mg/kg, or about 3-7 mg/kg. In some embodiments, the daily dosage of abemaciclib or a salt thereof is in the amount of about 100 mg, about 200 mg, about 300 mg, or about 400 mg. In some embodiments, the daily dosage of abemaciclib or a salt thereof is in the amount of about 50 mg twice daily, about 100 mg twice daily, about 150 mg twice daily, or about 200 mg twice daily. In some embodiments, the daily dosage of abemaciclib or a salt thereof is in the amount of less than about 300 mg, or less than about 400 mg. In some embodiments, the daily dosage of abemaciclib or a salt thereof is in the amount of about 100-400 mg. In some embodiments, the daily dosage of abemaciclib or a salt thereof is in the amount of about 4-6 mg/kg. In some embodiments, abemaciclib is in its mesylate salt form. The amounts of the inhibitor described herein and throughout the specification refer to the amount of the inhibitor without taking into consideration of the weight of the counterions if the inhibitor exists in a salt form. For example, the term “80 mg of abemaciclib” could include, without limitation, 80 mg of abemaciclib in a salt-free form or 95.3 mg of abemaciclib mesylate.

In some embodiments, the daily dosage of abemaciclib or a salt thereof is in the amount that is about at greater than about 20%, or greater than about 25%, or greater than about 30%, or greater than about 35%, or greater than about 40%, or greater than about 45%, or greater than about 50%, or greater than about 55%, or greater than about 60%, or greater than about 65%, or greater than about 70%, or greater than about 75%, or greater than about 80%, or greater than about 85%, or greater than about 90%, or greater than 95% by weight of the combined weekly dosage of cetuximab, cobimetinib, and abemaciclib or salts of the foregoing. In some embodiments, the daily dosage of abemaciclib or a salt thereof is in the amount that is about at less than 20%, or less than about 25%, or less than about 30%, or less than about 35%, or less than about 40%, or less than about 45%, or less than about 50%, or less than about 55%, or less than about 60%, or less than about 65%, or less than about 70%, or less than about 75%, or less than about 80%, or less than about 85%, or less than about 90%, or less than 95% by weight of the combined weekly dosage of cetuximab, cobimetinib, and abemaciclib or salts of the foregoing.

In another aspect, the method comprises administering an effective amount of cetuximab, binimetinib or a salt thereof, and palbociclib or a salt thereof. In some embodiments, the daily dosage of binimetinib or a salt thereof is in the amount of about 0.25-0.5 mg/kg, about 0.5-1 mg/kg, about 1-1.5 mg/kg, about 1.5-2 mg/kg, about 2-2.5 mg/kg, about 2.5-3 mg/kg, about 3-3.5 mg/kg, about 3.5-4 mg/kg, or about 0.5-3 mg/kg. In some embodiments, the daily dosage of binimetinib or a salt thereof is in the amount of about 60 mg, or about 90 mg. In some embodiments, the daily dosage of binimetinib or a salt thereof is in the amount of less than about 60 mg, or about 90 mg. In some embodiments, the dosage of binimetinib or a salt thereof is in the amount of about 60 mg, or about 90 mg. In some embodiments, the daily dosage of binimetinib or a salt thereof is in the amount of about 0.8-2.7 mg/kg.

In some embodiments, the daily dosage of binimetinib or a salt thereof is in the amount that is about at greater than about 20%, or greater than about 25%, or greater than about 30%, or greater than about 35%, or greater than about 40%, or greater than about 45%, or greater than about 50%, or greater than about 55%, or greater than about 60%, or greater than about 65%, or greater than about 70%, or greater than about 75%, or greater than about 80%, or greater than about 85%, or greater than about 90%, or greater than 95% by weight of the combined weekly dosage of cetuximab, binimetinib, and palbociclib or salts of the foregoing. In some embodiments, the daily dosage of binimetinib or a salt thereof is in the amount that is about at less than 20%, or less than about 25%, or less than about 30%, or less than about 35%, or less than about 40%, or less than about 45%, or less than about 50%, or less than about 55%, or less than about 60%, or less than about 65%, or less than about 70%, or less than about 75%, or less than about 80%, or less than about 85%, or less than about 90%, or less than 95% by weight of the combined weekly dosage of cetuximab, binimetinib, and palbociclib or salts of the foregoing. In another aspect, the method comprises administering an effective amount of osimertinib or a salt thereof, binimetinib or a salt thereof, and palbociclib or a salt thereof. In some embodiments, the daily dosage of binimetinib or a salt thereof is in the amount of about 0.25-0.5 mg/kg, about 0.5-1 mg/kg, about 1-1.5 mg/kg, about 1.5-2 mg/kg, about 2-2.5 mg/kg, about 2.5-3 mg/kg, about 3-3.5 mg/kg, about 3.5-4 mg/kg, or about 0.5-3 mg/kg. In some embodiments, the daily dosage of binimetinib or a salt thereof is in the amount of about 60 mg, or about 90 mg. In some embodiments, the daily dosage of binimetinib or a salt thereof is in the amount of less than about 60 mg, or about 90 mg. In some embodiments, the dosage of binimetinib or a salt thereof is in the amount of about 60 mg, or about 90 mg. In some embodiments, the daily dosage of binimetinib or a salt thereof is in the amount of about 0.8-2.7 mg/kg.

In another aspect, the method comprises administering an effective amount of osimertinib or a salt thereof, binimetinib or a salt thereof, and palbociclib or a salt thereof. In some embodiments, the daily dosage of binimetinib or a salt thereof is in the amount of about 0.25-0.5 mg/kg, about 0.5-1 mg/kg, about 1-1.5 mg/kg, about 1.5-2 mg/kg, about 2-2.5 mg/kg, about 2.5-3 mg/kg, about 3-3.5 mg/kg, about 3.5-4 mg/kg, or about 0.5-3 mg/kg. In some embodiments, the daily dosage of binimetinib or a salt thereof is in the amount of about 60 mg, or about 90 mg. In some embodiments, the daily dosage of binimetinib or a salt thereof is in the amount of less than about 60 mg, or about 90 mg. In some embodiments, the dosage of binimetinib or a salt thereof is in the amount of about 60 mg, or about 90 mg. In some embodiments, the daily dosage of binimetinib or a salt thereof is in the amount of about 0.8-2.7 mg/kg.

In some embodiments, the daily dosage of binimetinib or a salt thereof is in the amount that is about at greater than about 20%, or greater than about 25%, or greater than about 30%, or greater than about 35%, or greater than about 40%, or greater than about 45%, or greater than about 50%, or greater than about 55%, or greater than about 60%, or greater than about 65%, or greater than about 70%, or greater than about 75%, or greater than about 80%, or greater than about 85%, or greater than about 90%, or greater than 95% by weight of the combined weekly dosage of osimertinib or a salt thereof, binimetinib, and palbociclib or salts of the foregoing. In some embodiments, the daily dosage of binimetinib or a salt thereof is in the amount that is about at less than 20%, or less than about 25%, or less than about 30%, or less than about 35%, or less than about 40%, or less than about 45%, or less than about 50%, or less than about 55%, or less than about 60%, or less than about 65%, or less than about 70%, or less than about 75%, or less than about 80%, or less than about 85%, or less than about 90%, or less than 95% by weight of the combined weekly dosage of osimertinib or a salt thereof, binimetinib, and palbociclib or salts of the foregoing. In another aspect, the method comprises administering an effective amount of osimertinib or a salt thereof, binimetinib or a salt thereof, and palbociclib or a salt thereof. In some embodiments, the daily dosage of binimetinib or a salt thereof is in the amount of about 0.25-0.5 mg/kg, about 0.5-1 mg/kg, about 1-1.5 mg/kg, about 1.5-2 mg/kg, about 2-2.5 mg/kg, about 2.5-3 mg/kg, about 3-3.5 mg/kg, about 3.5-4 mg/kg, or about 0.5-3 mg/kg. In some embodiments, the daily dosage of binimetinib or a salt thereof is in the amount of about 60 mg, or about 90 mg. In some embodiments, the daily dosage of binimetinib or a salt thereof is in the amount of less than about 60 mg, or about 90 mg. In some embodiments, the dosage of binimetinib or a salt thereof is in the amount of about 60 mg, or about 90 mg. In some embodiments, the daily dosage of binimetinib or a salt thereof is in the amount of about 0.8-2.7 mg/kg.

In another aspect, the method comprises administering an effective amount of cetuximab, cobimetinib or a salt thereof, and palbociclib or a salt thereof. In some embodiments, the dosage of cetuximab is in the amount of about 0.1-20 mg/kg, about 0.1-0.5 mg/kg, about 0.5-1 mg/kg, about 1-2 mg/kg, about 2-3 mg/kg, about 3-5 mg/kg, about 5-7.5 mg/kg, about 7.5-10 mg/kg, about 10-15 mg/kg, about 15-20 mg/kg, or about 0.1-10 mg/kg. In some embodiments, the dosage of cetuximab is about 150-200 mg/m², about 200-250 mg/m², about 250-300 mg/m², about 300-400 mg/m², about 400-500 mg/m², about 500-750 mg/m², about 150-250 mg/m², about 250-400 mg/m², or about 400-750 mg/m². In some embodiments, cetuximab is infused over 30-180 minutes, about 30-60 minutes, about 60-120 minutes, or about 120-180 minutes. In some embodiments, the maximum infusion rate is about 5 mL/min or about 10 mL/min. In some embodiments, cetuximab is administered about every day, about every week, about every two weeks, about every three week, or about every four weeks. In some embodiments, the dosage of cetuximab is about 500 mg/m² infused over 60-120 minutes every two weeks. In some embodiments, cetuximab is administered in accordance with a schedule comprising an initial dose followed by several subsequent doses. In some embodiments, the initial dose is about 250-500 mg/m², about 250-300 mg/m², about 300-400 mg/m² or about 400-500 mg/m². In some embodiments, the initial dose is about 250 mg/m², about 400 mg/m² or about 500 mg/m². In some embodiments, the subsequent dose is about 50-300 mg/m², about 50-150 mg/m², about 150-200 mg/m² or about 200-300 mg/m². In some embodiments, the subsequent dose is about 50 mg/m², about 150 mg/m² or about 250 mg/m². In some embodiments, cetuximab is administered in 400 mg/m² infused over 120 minutes followed by 250 mg/m² weekly infused over 60 minutes. In some embodiments, cetuximab is administered in 400 mg/m² infused over 120 minutes followed by 150 mg/m² weekly infused over 60 minutes.

In some embodiments, the daily dosage of cobimetinib or a salt thereof is in the amount of about 0.1-0.25 mg/kg, about 0.25-0.5 mg/kg, about 0.5-0.75 mg/kg, about 0.75-1 mg/kg, about 1-1.25 mg/kg, about 1.25-1.5 mg/kg, about 1.5-1.75 mg/kg, about 1.75-2 mg/kg, or about 0.25-1 mg/kg. In some embodiments, the daily dosage of cobimetinib or a salt thereof is in the amount of about 20 mg, about 40 mg, or about 60 mg. In some embodiments, the daily dosage of cobimetinib or a salt thereof is in the amount of less than about 20 mg, about 40 mg, or about 60 mg. In some embodiments, the daily dosage of cobimetinib or a salt thereof is in the amount of about 20-60 mg. In some embodiments, the daily dosage of cobimetinib or a salt thereof is in the amount of about 0.3-1 mg/kg. In some embodiments, the cobimetinib is in its hemifumarate salt form.

In some embodiments, the daily dosage of palbociclib or a salt thereof is in the amount of about 0.25-0.5 mg/kg, about 0.5-1 mg/kg, about 1.5-2 mg/kg, about 2-2.5 mg/kg, about 2.5-3 mg/kg, about 3-3.5 mg/kg, about 1-2.5 mg/kg, about 1-3 mg/kg, or about 3-5 mg/kg. In some embodiments, the daily dosage of palbociclib or a salt thereof is in the amount of about 15 mg, about 50 mg, about 75 mg, about 100 mg, or about 125 mg. In some embodiments, the daily dosage of palbociclib or a salt thereof is in the amount of less than about 15 mg, about 50 mg, about 75 mg, about 100 mg, or about 125 mg. In some embodiments, the daily dosage of palbociclib or a salt thereof is in the amount of about 15-125 mg. In some embodiments, the daily dosage of palbociclib or a salt thereof is in the amount of about 0.25-2.5 mg/kg. In some embodiments, the method comprises administration of cetuximab, cobimetinib or a solvate or a salt thereof, and palbociclib or a solvate or a salt thereof.

In another aspect, the method comprises administering an effective amount of cetuximab, TAK-733 or a salt thereof and palbociclib or a salt thereof. In some embodiments, the daily dosage of TAK-733 or a salt thereof is in the amount of about 0.001-1 mg/kg, about 0.001-0.002 mg/kg, about 0.002-0.005 mg/kg, about 0.005-0.01 mg/kg, about 0.01-0.05 mg/kg, about 0.05-0.1 mg/kg, about 0.1-0.2 mg/kg, about 0.2-0.3 mg/kg, about 0.3-0.4 mg/kg, about 0.4-0.5 mg/kg, about 0.5-0.6 mg/kg, about 0.6-0.7 mg/kg, about 0.7-0.8 mg/kg, about 0.8-0.9 mg/kg, or about 0.9-1 mg/kg of TAK-733. In some embodiments, the daily dosage of TAK-733 or a salt thereof is in the amount of about 10 mg, about 15 mg, about 20 mg, or about 25 mg of TAK-733. In some embodiments, the daily dosage of TAK-733 or a salt thereof is in the amount of less than about 10 mg, about 15 mg, about 20 mg, or about 25 mg of TAK-733. In some embodiments, the daily dosage of TAK-733 or a salt thereof is in the amount of about 0.1-25 mg, about 0.1-1 mg, about 1-5 mg, about 5-10 mg, about 8-16 mg, about 10-15 mg, about 15-20 mg, or about 20-25 mg of TAK-733.

In some embodiments, the dosage of cetuximab is in the amount of about 0.1-20 mg/kg, about 0.1-0.5 mg/kg, about 0.5-1 mg/kg, about 1-2 mg/kg, about 2-3 mg/kg, about 3-5 mg/kg, about 5-7.5 mg/kg, about 7.5-10 mg/kg, about 10-15 mg/kg, about 15-20 mg/kg, or about 0.1-10 mg/kg. In some embodiments, the dosage of cetuximab is about 150-200 mg/m², about 200-250 mg/m², about 250-300 mg/m², about 300-400 mg/m², about 400-500 mg/m², about 500-750 mg/m², about 150-250 mg/m², about 250-400 mg/m², or about 400-750 mg/m². In some embodiments, cetuximab is infused over 30-180 minutes, about 30-60 minutes, about 60-120 minutes, or about 120-180 minutes. In some embodiments, the maximum infusion rate is about 5 mL/min or about 10 mL/min. In some embodiments, cetuximab is administered about every day, about every week, about every two weeks, about every three week, or about every four weeks. In some embodiments, the dosage of cetuximab is about 500 mg/m² infused over 60-120 minutes every two weeks. In some embodiments, cetuximab is administered in accordance with a schedule comprising an initial dose followed by several subsequent doses. In some embodiments, the initial dose is about 250-500 mg/m², about 250-300 mg/m², about 300-400 mg/m² or about 400-500 mg/m². In some embodiments, the initial dose is about 250 mg/m², about 400 mg/m² or about 500 mg/m². In some embodiments, the subsequent dose is about 50-300 mg/m², about 50-150 mg/m², about 150-200 mg/m² or about 200-300 mg/m². In some embodiments, the subsequent dose is about 50 mg/m², about 150 mg/m² or about 250 mg/m². In some embodiments, cetuximab is administered in 400 mg/m² infused over 120 minutes followed by 250 mg/m² weekly infused over 60 minutes. In some embodiments, cetuximab is administered in 400 mg/m² infused over 120 minutes followed by 150 mg/m² weekly infused over 60 minutes.

In some embodiments, the daily dosage of palbociclib or a salt thereof is in the amount of about 0.25-0.5 mg/kg, about 0.5-1 mg/kg, about 1.5-2 mg/kg, about 2-2.5 mg/kg, about 2.5-3 mg/kg, about 3-3.5 mg/kg, about 1-2.5 mg/kg, about 1-3 mg/kg, or about 3-5 mg/kg. In some embodiments, the daily dosage of palbociclib or a salt thereof is in the amount of about 15 mg, about 50 mg, about 75 mg, about 100 mg, or about 125 mg. In some embodiments, the daily dosage of palbociclib or a salt thereof is in the amount of less than about 15 mg, about 50 mg, about 75 mg, about 100 mg, or about 125 mg. In some embodiments, the daily dosage of palbociclib or a salt thereof is in the amount of about 15-125 mg. In some embodiments, the daily dosage of palbociclib or a salt thereof is in the amount of about 0.25-2.5 mg/kg. In some embodiments, the method comprises administration of cetuximab, TAK-733 or a solvate or a salt thereof, and palbociclib or a solvate or a salt thereof.

In another aspect, the method comprises administering an effective amount of osimertinib or a salt thereof, TAK-733 or a salt thereof, and palbociclib or a salt thereof. In some embodiments, the daily dosage of osimertinib or a salt thereof is in the amount of about 0.25-0.5 mg/kg, about 0.5-1 mg/kg, about 1-1.5 mg/kg, about 1.5-2 mg/kg, about 2-2.5 mg/kg, about 2.5-3 mg/kg, about 3-3.5 mg/kg, about 3.5-4 mg/kg, or about 0.5-3 mg/kg of osimertinib. In some embodiments, the daily dosage of osimertinib or a salt thereof is in the amount of about 20 mg, about 40 mg, about 80 mg, or about 160 mg of osimertinib. In some embodiments, the daily dosage of osimertinib or a salt thereof is in the amount of less than about 20 mg, about 40 mg, about 80 mg, or about 160 mg of osimertinib. In some embodiments, the dosage of osimertinib or a salt thereof is in the amount of about 20-240 mg, about 20-40 mg, about 40-80 mg, about 80-160 mg, about 160-240 mg or about 40-160 mg of osimertinib. In some embodiments, the daily dosage of osimertinib or a salt thereof is in the amount of about 0.6-2.7 mg/kg of osimertinib. In some embodiments, osimertinib is in its mesylate salt form.

In some embodiments, the daily dosage of TAK-733 or a salt thereof is in the amount of about 0.001-1 mg/kg, about 0.001-0.002 mg/kg, about 0.002-0.005 mg/kg, about 0.005-0.01 mg/kg, about 0.01-0.05 mg/kg, about 0.05-0.1 mg/kg, about 0.1-0.2 mg/kg, about 0.2-0.3 mg/kg, about 0.3-0.4 mg/kg, about 0.4-0.5 mg/kg, about 0.5-0.6 mg/kg, about 0.6-0.7 mg/kg, about 0.7-0.8 mg/kg, about 0.8-0.9 mg/kg, or about 0.9-1 mg/kg of TAK-733. In some embodiments, the daily dosage of TAK-733 or a salt thereof is in the amount of about 10 mg, about 15 mg, about 20 mg, or about 25 mg of TAK-733. In some embodiments, the daily dosage of TAK-733 or a salt thereof is in the amount of less than about 10 mg, about 15 mg, about 20 mg, or about 25 mg of TAK-733. In some embodiments, the daily dosage of TAK-733 or a salt thereof is in the amount of about 0.1-25 mg, about 0.1-1 mg, about 1-5 mg, about 5-10 mg, about 8-16 mg, about 10-15 mg, about 15-20 mg, or about 20-25 mg of TAK-733.

In some embodiments, the daily dosage of palbociclib or a salt thereof is in the amount of about 0.25-0.5 mg/kg, about 0.5-1 mg/kg, about 1.5-2 mg/kg, about 2-2.5 mg/kg, about 2.5-3 mg/kg, about 3-3.5 mg/kg, about 1-2.5 mg/kg, about 1-3 mg/kg, or about 3-5 mg/kg of palbociclib. In some embodiments, the daily dosage of palbociclib or a salt thereof is in the amount of about 15 mg, about 50 mg, about 50 mg, about 75 mg, about 100 mg, or about 125 mg of palbociclib. In some embodiments, the daily dosage of palbociclib or a salt thereof is in the amount of less than about 15 mg, about 50 mg, about 50 mg, about 75 mg, about 100 mg, or about 125 mg of palbociclib. In some embodiments, the daily dosage of palbociclib or a salt thereof is in the amount of about 15-225 mg, about 25-50 mg, about 50-75 mg, about 75-125 mg, about 125-150 mg about 150-200 mg, or about 200-225 mg of palbociclib. In some embodiments, the daily dosage of palbociclib or a salt thereof is in the amount of about 0.25-2.5 mg/kg of palbociclib. In some embodiments, the method comprises administration of osimertinib or a solvate or a salt thereof, TAK-733 or a solvate or a salt thereof, and palbociclib or a solvate or a salt thereof.

In another aspect, the method comprises administering an effective amount of osimertinib or a salt thereof, TAK-733 or a salt thereof, and palbociclib or a salt thereof. In some embodiments, the daily dosage of osimertinib or a salt thereof is in the amount of about 0.25-0.5 mg/kg, about 0.5-1 mg/kg, about 1-1.5 mg/kg, about 1.5-2 mg/kg, about 2-2.5 mg/kg, about 2.5-3 mg/kg, about 3-3.5 mg/kg, about 3.5-4 mg/kg, or about 0.5-3 mg/kg of osimertinib. In some embodiments, the daily dosage of osimertinib or a salt thereof is in the amount of about 20 mg, about 40 mg, about 80 mg, or about 160 mg of osimertinib. In some embodiments, the daily dosage of osimertinib or a salt thereof is in the amount of less than about 20 mg, about 40 mg, about 80 mg, or about 160 mg of osimertinib. In some embodiments, the dosage of osimertinib or a salt thereof is in the amount of about 20-240 mg, about 20-40 mg, about 40-80 mg, about 80-160 mg, about 160-240 mg or about 40-160 mg of osimertinib. In some embodiments, the daily dosage of osimertinib or a salt thereof is in the amount of about 0.6-2.7 mg/kg of osimertinib. In some embodiments, osimertinib is in its mesylate salt form.

In some embodiments, the daily dosage of TAK-733 or a salt thereof is in the amount of about 0.001-1 mg/kg, about 0.001-0.002 mg/kg, about 0.002-0.005 mg/kg, about 0.005-0.01 mg/kg, about 0.01-0.05 mg/kg, about 0.05-0.1 mg/kg, about 0.1-0.2 mg/kg, about 0.2-0.3 mg/kg, about 0.3-0.4 mg/kg, about 0.4-0.5 mg/kg, about 0.5-0.6 mg/kg, about 0.6-0.7 mg/kg, about 0.7-0.8 mg/kg, about 0.8-0.9 mg/kg, or about 0.9-1 mg/kg of TAK-733. In some embodiments, the daily dosage of TAK-733 or a salt thereof is in the amount of about 10 mg, about 15 mg, about 20 mg, or about 25 mg of TAK-733. In some embodiments, the daily dosage of TAK-733 or a salt thereof is in the amount of less than about 10 mg, about 15 mg, about 20 mg, or about 25 mg of TAK-733. In some embodiments, the daily dosage of TAK-733 or a salt thereof is in the amount of about 0.1-25 mg, about 0.1-1 mg, about 1-5 mg, about 5-10 mg, about 8-16 mg, about 10-15 mg, about 15-20 mg, or about 20-25 mg of TAK-733.

In some embodiments, the daily dosage of palbociclib or a salt thereof is in the amount of about 0.25-0.5 mg/kg, about 0.5-1 mg/kg, about 1.5-2 mg/kg, about 2-2.5 mg/kg, about 2.5-3 mg/kg, about 3-3.5 mg/kg, about 1-2.5 mg/kg, about 1-3 mg/kg, or about 3-5 mg/kg of palbociclib. In some embodiments, the daily dosage of palbociclib or a salt thereof is in the amount of about 15 mg, about 50 mg, about 50 mg, about 75 mg, about 100 mg, or about 125 mg of palbociclib. In some embodiments, the daily dosage of palbociclib or a salt thereof is in the amount of less than about 15 mg, about 50 mg, about 50 mg, about 75 mg, about 100 mg, or about 125 mg of palbociclib. In some embodiments, the daily dosage of palbociclib or a salt thereof is in the amount of about 15-225 mg, about 25-50 mg, about 50-75 mg, about 75-125 mg, about 125-150 mg about 150-200 mg, or about 200-225 mg of palbociclib. In some embodiments, the daily dosage of palbociclib or a salt thereof is in the amount of about 0.25-2.5 mg/kg of palbociclib. In some embodiments, the method comprises administration of osimertinib or a solvate or a salt thereof, TAK-733 or a solvate or a salt thereof, and palbociclib or a solvate or a salt thereof.

In another aspect, the method comprises administering an effective amount of cetuximab, trametinib or a salt thereof and palbociclib or a salt thereof. In some embodiments, the daily dosage of trametinib or a salt thereof is in the amount of about 0.01-1 mg/kg, about 0.01-0.02 mg/kg, about 0.02-0.03 mg/kg, about 0.03-0.05 mg/kg, about 0.05-0.08 mg/kg, about 0.08-0.1 mg/kg, about 0.1-0.2 mg/kg, about 0.2-0.3 mg/kg, about 0.3-0.4 mg/kg, about 0.4-0.5 mg/kg, about 0.5-0.6 mg/kg, about 0.6-0.7 mg/kg, about 0.7-0.8 mg/kg, about 0.8-0.9 mg/kg, or about 0.9-1 mg/kg of trametinib. In some embodiments, the daily dosage of trametinib or a salt thereof is in the amount of about 0.5 mg, about 1 mg, about 2 mg, or about 4 mg of trametinib. In some embodiments, the daily dosage of trametinib or a salt thereof is in the amount of less than about 0.5 mg, about 1 mg, about 2 mg, or about 4 mg of trametinib. In some embodiments, the daily dosage of trametinib or a salt thereof is in the amount of about 0.1-25 mg, about 0.1-0.5 mg, about 0.5-1 mg, about 1-2 mg, about 2-4 mg, about 4-10 mg or about 0.5-2 mg of trametinib.

In some embodiments, the dosage of cetuximab is in the amount of about 0.1-20 mg/kg, about 0.1-0.5 mg/kg, about 0.5-1 mg/kg, about 1-2 mg/kg, about 2-3 mg/kg, about 3-5 mg/kg, about 5-7.5 mg/kg, about 7.5-10 mg/kg, about 10-15 mg/kg, about 15-20 mg/kg, or about 0.1-10 mg/kg. In some embodiments, the dosage of cetuximab is about 150-200 mg/m², about 200-250 mg/m², about 250-300 mg/m², about 300-400 mg/m², about 400-500 mg/m², about 500-750 mg/m², about 150-250 mg/m², about 250-400 mg/m², or about 400-750 mg/m². In some embodiments, cetuximab is infused over 30-180 minutes, about 30-60 minutes, about 60-120 minutes, or about 120-180 minutes. In some embodiments, the maximum infusion rate is about 5 mL/min or about 10 mL/min. In some embodiments, cetuximab is administered about every day, about every week, about every two weeks, about every three week, or about every four weeks. In some embodiments, the dosage of cetuximab is about 500 mg/m² infused over 60-120 minutes every two weeks. In some embodiments, cetuximab is administered in accordance with a schedule comprising an initial dose followed by several subsequent doses. In some embodiments, the initial dose is about 250-500 mg/m², about 250-300 mg/m², about 300-400 mg/m² or about 400-500 mg/m². In some embodiments, the initial dose is about 250 mg/m², about 400 mg/m² or about 500 mg/m². In some embodiments, the subsequent dose is about 50-300 mg/m², about 50-150 mg/m², about 150-200 mg/m² or about 200-300 mg/m². In some embodiments, the subsequent dose is about 50 mg/m², about 150 mg/m² or about 250 mg/m². In some embodiments, cetuximab is administered in 400 mg/m² infused over 120 minutes followed by 250 mg/m² weekly infused over 60 minutes. In some embodiments, cetuximab is administered in 400 mg/m² infused over 120 minutes followed by 150 mg/m² weekly infused over 60 minutes.

In some embodiments, the daily dosage of palbociclib or a salt thereof is in the amount of about 0.25-0.5 mg/kg, about 0.5-1 mg/kg, about 1.5-2 mg/kg, about 2-2.5 mg/kg, about 2.5-3 mg/kg, about 3-3.5 mg/kg, about 1-2.5 mg/kg, about 1-3 mg/kg, or about 3-5 mg/kg of palbociclib. In some embodiments, the daily dosage of palbociclib or a salt thereof is in the amount of about 15 mg, about 50 mg, about 50 mg, about 75 mg, about 100 mg, or about 125 mg of palbociclib. In some embodiments, the daily dosage of palbociclib or a salt thereof is in the amount of less than about 15 mg, about 50 mg, about 50 mg, about 75 mg, about 100 mg, or about 125 mg of palbociclib. In some embodiments, the daily dosage of palbociclib or a salt thereof is in the amount of about 15-225 mg, about 25-50 mg, about 50-75 mg, about 75-125 mg, about 125-150 mg about 150-200 mg, or about 200-225 mg of palbociclib. In some embodiments, the daily dosage of palbociclib or a salt thereof is in the amount of about 0.25-2.5 mg/kg of palbociclib. In some embodiments, the method comprises administration of cetuximab, TAK-733 or a solvate or a salt thereof, and palbociclib or a solvate or a salt thereof.

In another aspect, the method comprises administering an effective amount of osimertinib or a salt thereof, trametinib or a salt thereof, and palbociclib or a salt thereof. In some embodiments, the daily dosage of osimertinib or a salt thereof is in the amount of about 0.25-0.5 mg/kg, about 0.5-1 mg/kg, about 1-1.5 mg/kg, about 1.5-2 mg/kg, about 2-2.5 mg/kg, about 2.5-3 mg/kg, about 3-3.5 mg/kg, about 3.5-4 mg/kg, or about 0.5-3 mg/kg of osimertinib. In some embodiments, the daily dosage of osimertinib or a salt thereof is in the amount of about 20 mg, about 40 mg, about 80 mg, or about 160 mg of osimertinib. In some embodiments, the daily dosage of osimertinib or a salt thereof is in the amount of less than about 20 mg, about 40 mg, about 80 mg, or about 160 mg of osimertinib. In some embodiments, the dosage of osimertinib or a salt thereof is in the amount of about 20-240 mg, about 20-40 mg, about 40-80 mg, about 80-160 mg, about 160-240 mg or about 40-160 mg of osimertinib. In some embodiments, the daily dosage of osimertinib or a salt thereof is in the amount of about 0.6-2.7 mg/kg of osimertinib. In some embodiments, osimertinib is in its mesylate salt form.

In some embodiments, the daily dosage of trametinib or a salt thereof is in the amount of about 0.01-1 mg/kg, about 0.01-0.02 mg/kg, about 0.02-0.03 mg/kg, about 0.03-0.05 mg/kg, about 0.05-0.08 mg/kg, about 0.08-0.1 mg/kg, about 0.1-0.2 mg/kg, about 0.2-0.3 mg/kg, about 0.3-0.4 mg/kg, about 0.4-0.5 mg/kg, about 0.5-0.6 mg/kg, about 0.6-0.7 mg/kg, about 0.7-0.8 mg/kg, about 0.8-0.9 mg/kg, or about 0.9-1 mg/kg of trametinib. In some embodiments, the daily dosage of trametinib or a salt thereof is in the amount of about 0.5 mg, about 1 mg, about 2 mg, or about 4 mg of trametinib. In some embodiments, the daily dosage of trametinib or a salt thereof is in the amount of less than about 0.5 mg, about 1 mg, about 2 mg, or about 4 mg of trametinib. In some embodiments, the daily dosage of trametinib or a salt thereof is in the amount of about 0.1-25 mg, about 0.1-0.5 mg, about 0.5-1 mg, about 1-2 mg, about 2-4 mg, about 4-10 mg or about 0.5-2 mg of trametinib.

In some embodiments, the daily dosage of palbociclib or a salt thereof is in the amount of about 0.25-0.5 mg/kg, about 0.5-1 mg/kg, about 1.5-2 mg/kg, about 2-2.5 mg/kg, about 2.5-3 mg/kg, about 3-3.5 mg/kg, about 1-2.5 mg/kg, about 1-3 mg/kg, or about 3-5 mg/kg of palbociclib. In some embodiments, the daily dosage of palbociclib or a salt thereof is in the amount of about 15 mg, about 50 mg, about 50 mg, about 75 mg, about 100 mg, or about 125 mg of palbociclib. In some embodiments, the daily dosage of palbociclib or a salt thereof is in the amount of less than about 15 mg, about 50 mg, about 50 mg, about 75 mg, about 100 mg, or about 125 mg of palbociclib. In some embodiments, the daily dosage of palbociclib or a salt thereof is in the amount of about 15-225 mg, about 25-50 mg, about 50-75 mg, about 75-125 mg, about 125-150 mg about 150-200 mg, or about 200-225 mg of palbociclib. In some embodiments, the daily dosage of palbociclib or a salt thereof is in the amount of about 0.25-2.5 mg/kg of palbociclib. In some embodiments, the method comprises administration of osimertinib or a solvate or a salt thereof, trametinib or a solvate or a salt thereof, and palbociclib or a solvate or a salt thereof.

In another aspect, the method provides administering an effective amount of an EGFR inhibitor (e.g., osimertinib, lapatinib, or cetuximab), a MEK 1/2 inhibitor (e.g., cobimetinib, trametinib, binimetinib, or TAK-733) and a CDK 4/6 inhibitor (e.g., palbociclib or abemaciclib) until disease progression or unacceptable toxicity. In some embodiments, the method provides administering an effective amount of an EGFR inhibitor (e.g., osimertinib, lapatinib, or cetuximab), a MEK 1/2 inhibitor (e.g., cobimetinib, trametinib, binimetinib, or TAK-733) and a CDK 4/6 inhibitor (e.g., palbociclib or abemaciclib) for at least about 1-2 weeks, about 2-3 weeks, about 3-4 weeks, about 4-5 weeks, about 5-6 weeks, about 6-7 weeks, about 7-8 weeks, about 8-9 weeks, about 9-10 weeks, about 2-3 months, about 3-4 months, about 4-5 months, about 5-6 months, about 6-12 months, or about 12-24 months. In some embodiments, the effective amount of an EGFR inhibitor (e.g., osimertinib, lapatinib, or cetuximab), a MEK 1/2 inhibitor (e.g., cobimetinib, trametinib, binimetinib, or TAK-733) and a CDK 4/6 inhibitor (e.g., palbociclib or abemaciclib) is administered for 21 consecutive days followed by 7 days off to comprise a complete cycle of 28 days. In some embodiments, the subject is administered an effective amount of an EGFR inhibitor (e.g., osimertinib, lapatinib, or cetuximab), a MEK 1/2 inhibitor (e.g., cobimetinib, trametinib, binimetinib, or TAK-733) and a CDK 4/6 inhibitor (e.g., palbociclib or abemaciclib) for one, two, three, four, five, six, seven, eight, nine, ten or more cycles of 28 days. In some embodiments, the EGFR inhibitor (e.g., osimertinib, lapatinib, or cetuximab), the MEK 1/2 inhibitor (e.g., cobimetinib, trametinib, binimetinib, or TAK-733) and the CDK 4/6 inhibitor (e.g., palbociclib or abemaciclib) are administered for different durations.

In another aspect, the effective amount of an EGFR inhibitor (e.g., osimertinib, lapatinib, or cetuximab), a MEK 1/2 inhibitor (e.g., cobimetinib, trametinib, binimetinib, or TAK-733) and a CDK 4/6 inhibitor (e.g., palbociclib or abemaciclib) are formulated as one composition. In another aspect, the effective amount of an EGFR inhibitor (e.g., osimertinib, lapatinib, or cetuximab), a MEK 1/2 inhibitor (e.g., cobimetinib, trametinib, binimetinib, or TAK-733) and a CDK 4/6 inhibitor (e.g., palbociclib or abemaciclib) are formulated separately. In some embodiments, osimertinib or a salt thereof, cobimetinib or a salt thereof, and palbociclib or a salt thereof are formulated as one composition or separate compositions for oral administration. In some embodiments, osimertinib or a salt thereof, TAK-733 or a salt thereof, and palbociclib or a salt thereof are formulated as one composition or separate compositions for oral administration. In some embodiments, lapatinib or a salt thereof, trametinib or a salt thereof, and palbociclib or a salt thereof are formulated as one composition or separate compositions for oral administration. In some embodiments, osimertinib or a salt thereof, binimetinib or a salt thereof, and palbociclib or a salt thereof are formulated as one composition or separate compositions for oral administration. In some embodiments, osimertinib or a salt thereof, trametinib or a salt thereof, and palbociclib or a salt thereof are formulated as one composition or separate compositions for oral administration. In some embodiments, cobimetinib or a salt thereof and palbociclib or a salt thereof are formulated as one composition or separate compositions for oral administration. In some embodiments, TAK-733 or a salt thereof and palbociclib or a salt thereof are formulated as one composition or separate compositions for oral administration. In some embodiments, trametinib or a salt thereof and palbociclib or a salt thereof are formulated as one composition or separate compositions for oral administration. For oral administration, the method may comprise formulating the compounds in a solid form, such as a tablet or capsule, or as a solution, emulsion, or suspension. To prepare the oral compositions, the compounds may be formulated to yield a daily dosage described herein. In some embodiments, cetuximab is formulated for intravenous infusion.

In some embodiments, the EGFR inhibitor (e.g., osimertinib, lapatinib, or cetuximab), the MEK 1/2 inhibitor (e.g., cobimetinib, trametinib, binimetinib, or TAK-733) and the CDK 4/6 inhibitor (e.g., palbociclib or abemaciclib) are formulated in the same form. In some embodiments, osimertinib or a salt thereof, cobimetinib or a salt thereof, and/or palbociclib or a salt thereof are formulated in a solid form, such as a tablet or capsule. In some embodiments, osimertinib or a salt thereof, TAK-733 or a salt thereof, and/or palbociclib or a salt thereof are formulated in a solid form, such as a tablet or capsule. In some embodiments, osimertinib or a salt thereof, trametinib or a salt thereof, and/or palbociclib or a salt thereof are formulated in a solid form, such as a tablet or capsule. In some embodiments, cetuximab, cobimetinib or a salt thereof, and/or palbociclib or a salt thereof are formulated in a liquid form, such as suspensions, solutions, emulsions, or syrups, or may be lyophilized. In some embodiments, cetuximab, TAK-733 or a salt thereof, and/or palbociclib or a salt thereof are formulated in a liquid form, such as suspensions, solutions, emulsions, or syrups, or may be lyophilized. In some embodiments, cetuximab, trametinib or a salt thereof, and/or palbociclib or a salt thereof are formulated in a liquid form, such as suspensions, solutions, emulsions, or syrups, or may be lyophilized. In some embodiments, lapatinib or a salt thereof, trametinib or a salt thereof, and/or palbociclib or a salt thereof are formulated in a liquid form, such as suspensions, solutions, emulsions, or syrups, or may be lyophilized. In some embodiments, osimertinib or a salt thereof, binimetinib or a salt thereof, and/or palbociclib or a salt thereof are formulated in a liquid form, such as suspensions, solutions, emulsions, or syrups, or may be lyophilized. In some embodiments, the EGFR inhibitor (e.g., osimertinib, lapatinib, or cetuximab), the MEK 1/2 inhibitor (e.g., cobimetinib, trametinib, binimetinib, or TAK-733) and the CDK 4/6 inhibitor (e.g., palbociclib or abemaciclib) are formulated in the different forms.

In another aspect, the EGFR inhibitor (e.g., osimertinib, lapatinib, or cetuximab), the MEK 1/2 inhibitor (e.g., cobimetinib, trametinib, binimetinib, or TAK-733) and the CDK 4/6 inhibitor (e.g., palbociclib or abemaciclib) are administered simultaneously or intermittently. In some embodiments, the EGFR inhibitor (e.g., osimertinib, lapatinib, or cetuximab), the MEK 1/2 inhibitor (e.g., cobimetinib, trametinib, binimetinib, or TAK-733) and the CDK 4/6 inhibitor (e.g., palbociclib or abemaciclib) are formulated as one composition and administered as one composition. In some embodiments, the EGFR inhibitor (e.g., osimertinib, lapatinib, or cetuximab), the MEK 1/2 inhibitor (e.g., cobimetinib, trametinib, binimetinib, or TAK-733) and the CDK 4/6 inhibitor (e.g., palbociclib or abemaciclib) are formulated separately and administered simultaneously. In some embodiments, the EGFR inhibitor (e.g., osimertinib, lapatinib, or cetuximab), the MEK 1/2 inhibitor (e.g., cobimetinib, trametinib, binimetinib, or TAK-733) and the CDK 4/6 inhibitor (e.g., palbociclib or abemaciclib) are formulated separately and administered intermittently. In some embodiments, the EGFR inhibitor (e.g., osimertinib, lapatinib, or cetuximab), the MEK 1/2 inhibitor (e.g., cobimetinib, trametinib, binimetinib, or TAK-733) and the CDK 4/6 inhibitor (e.g., palbociclib or abemaciclib) are formulated separately and administered with different dosing frequencies.

In another aspect, the method comprises administering an effective amount of osimertinib or a salt thereof, cobimetinib or a salt thereof, and palbociclib or a salt thereof. In some embodiments, osimertinib or a salt thereof is administered before cobimetinib or a salt thereof and palbociclib or a salt thereof. In some embodiments, cobimetinib or a salt thereof is administered before osimertinib or a salt thereof and palbociclib or a salt thereof. In some embodiments, palbociclib or a salt thereof is administered before osimertinib or a salt thereof and cobimetinib or a salt thereof. In some embodiments, osimertinib or a salt thereof and cobimetinib or a salt thereof are administered together in the same or separate compositions. In some embodiments, osimertinib or a salt thereof and palbociclib or a salt thereof are administered together in the same or separate compositions. In some embodiments, cobimetinib or a salt thereof and palbociclib or a salt thereof are administered together in the same or separate compositions.

In another aspect, the method comprises administering an effective amount of cetuximab, cobimetinib or a salt thereof, and palbociclib or a salt thereof. In some embodiments, cetuximab is administered before cobimetinib or a salt thereof and palbociclib or a salt thereof. In some embodiments, cobimetinib or a salt thereof is administered before cetuximab and palbociclib or a salt thereof. In some embodiments, palbociclib or a salt thereof is administered before cetuximab and cobimetinib or a salt thereof. In some embodiments, cetuximab and cobimetinib or a salt thereof are administered together in the same or separate compositions. In some embodiments, cetuximab and palbociclib or a salt thereof are administered together in the same or separate compositions. In some embodiments, cobimetinib or a salt thereof and palbociclib or a salt thereof are administered together in the same or separate compositions. In some embodiments, cetuximab is administered in a separate composition from cobimetinib or a salt thereof, and palbociclib or a salt thereof. In some embodiments, cetuximab is administered every week or every two weeks, while cobimetinib or a salt thereof and palbociclib or a salt thereof are administered on a daily basis.

In another aspect, the method comprises administering an effective amount of osimertinib or a salt thereof, TAK-733 or a salt thereof, and palbociclib or a salt thereof. In some embodiments, osimertinib or a salt thereof is administered before TAK-733 or a salt thereof and palbociclib or a salt thereof. In some embodiments, TAK-733 or a salt thereof is administered before osimertinib or a salt thereof and palbociclib or a salt thereof. In some embodiments, palbociclib or a salt thereof is administered before osimertinib or a salt thereof and TAK-733 or a salt thereof. In some embodiments, osimertinib or a salt thereof and TAK-733 or a salt thereof are administered together in the same or separate compositions. In some embodiments, osimertinib or a salt thereof and palbociclib or a salt thereof are administered together in the same or separate compositions. In some embodiments, TAK-733 or a salt thereof and palbociclib or a salt thereof are administered together in the same or separate compositions.

In another aspect, the method comprises administering an effective amount of cetuximab, TAK-733 or a salt thereof, and palbociclib or a salt thereof. In some embodiments, cetuximab is administered before TAK-733 or a salt thereof and palbociclib or a salt thereof. In some embodiments, TAK-733 or a salt thereof is administered before cetuximab and palbociclib or a salt thereof. In some embodiments, palbociclib or a salt thereof is administered before cetuximab and TAK-733 or a salt thereof. In some embodiments, cetuximab and TAK-733 or a salt thereof are administered together in the same or separate compositions. In some embodiments, cetuximab and palbociclib or a salt thereof are administered together in the same or separate compositions. In some embodiments, TAK-733 or a salt thereof and palbociclib or a salt thereof are administered together in the same or separate compositions. In some embodiments, cetuximab is administered in a separate composition from TAK-733 or a salt thereof, and palbociclib or a salt thereof. In some embodiments, cetuximab is administered every week or every two weeks, while TAK-733 or a salt thereof and palbociclib or a salt thereof are administered on a daily basis.

In another aspect, the method comprises administering an effective amount of cetuximab, trametinib or a salt thereof, and palbociclib or a salt thereof. In some embodiments, cetuximab is administered before trametinib or a salt thereof and palbociclib or a salt thereof. In some embodiments, trametinib or a salt thereof is administered before cetuximab and palbociclib or a salt thereof. In some embodiments, palbociclib or a salt thereof is administered before cetuximab and trametinib or a salt thereof. In some embodiments, cetuximab and trametinib or a salt thereof are administered together in the same or separate compositions. In some embodiments, cetuximab and palbociclib or a salt thereof are administered together in the same or separate compositions. In some embodiments, trametinib or a salt thereof and palbociclib or a salt thereof are administered together in the same or separate compositions. In some embodiments, cetuximab is administered in a separate composition from trametinib or a salt thereof and palbociclib or a salt thereof. In some embodiments, cetuximab is administered every week or every two weeks, while trametinib or a salt thereof and palbociclib or a salt thereof are administered on a daily basis.

In another aspect, the method comprises administering an effective amount of lapatinib or a salt thereof, trametinib or a salt thereof, and palbociclib or a salt thereof. In some embodiments, lapatinib or a salt thereof is administered before trametinib or a salt thereof and palbociclib or a salt thereof. In some embodiments, trametinib or a salt thereof is administered before lapatinib or a salt thereof and palbociclib or a salt thereof. In some embodiments, palbociclib or a salt thereof is administered before lapatinib or a salt thereof and trametinib or a salt thereof. In some embodiments, lapatinib or a salt thereof and trametinib or a salt thereof are administered together in the same or separate compositions. In some embodiments, lapatinib or a salt thereof and palbociclib or a salt thereof are administered together in the same or separate compositions. In some embodiments, trametinib or a salt thereof and palbociclib or a salt thereof are administered together in the same or separate compositions. In some embodiments, lapatinib or a salt thereof, trametinib or a salt thereof and palbociclib or a salt thereof are administered in the same composition. In some embodiments, lapatinib or a salt thereof, trametinib or a salt thereof and palbociclib or a salt thereof are administered on a daily basis.

In another aspect, the method comprises administering an effective amount of osimertinib or a salt thereof, binimetinib or a salt thereof, and palbociclib or a salt thereof. In some embodiments, osimertinib or a salt thereof is administered before binimetinib or a salt thereof and palbociclib or a salt thereof. In some embodiments, binimetinib or a salt thereof is administered before osimertinib or a salt thereof and palbociclib or a salt thereof. In some embodiments, palbociclib or a salt thereof is administered before osimertinib or a salt thereof and binimetinib or a salt thereof. In some embodiments, osimertinib or a salt thereof and binimetinib or a salt thereof are administered together in the same or separate compositions. In some embodiments, osimertinib or a salt thereof and palbociclib or a salt thereof are administered together in the same or separate compositions. In some embodiments, binimetinib or a salt thereof and palbociclib or a salt thereof are administered together in the same or separate compositions. In some embodiments, osimertinib or a salt thereof, binimetinib or a salt thereof and palbociclib or a salt thereof are administered in the same composition. In some embodiments, osimertinib or a salt thereof, binimetinib or a salt thereof and palbociclib or a salt thereof are administered on a daily basis.

In another aspect, the method comprises administering an effective amount of cetuximab, binimetinib or a salt thereof, and palbociclib or a salt thereof. In some embodiments, cetuximab is administered before binimetinib or a salt thereof and palbociclib or a salt thereof. In some embodiments, binimetinib or a salt thereof is administered before cetuximab and palbociclib or a salt thereof. In some embodiments, palbociclib or a salt thereof is administered before cetuximab and binimetinib or a salt thereof. In some embodiments, cetuximab and binimetinib or a salt thereof are administered together in the same or separate compositions. In some embodiments, cetuximab and palbociclib or a salt thereof are administered together in the same or separate compositions. In some embodiments, binimetinib or a salt thereof and palbociclib or a salt thereof are administered together in the same or separate compositions. In some embodiments, cetuximab, binimetinib or a salt thereof and palbociclib or a salt thereof are administered in the same composition. In some embodiments, cetuximab is administered every week or every two weeks, while binimetinib or a salt thereof and palbociclib or a salt thereof are administered on a daily basis.

In another aspect, the method comprises administering an effective amount of cetuximab, cobimetinib or a salt thereof, and abemaciclib or a salt thereof. In some embodiments, cetuximab is administered before cobimetinib or a salt thereof and abemaciclib or a salt thereof. In some embodiments, cobimetinib or a salt thereof is administered before cetuximab and abemaciclib or a salt thereof. In some embodiments, abemaciclib or a salt thereof is administered before cetuximab and cobimetinib or a salt thereof. In some embodiments, cetuximab and cobimetinib or a salt thereof are administered together in the same or separate compositions. In some embodiments, cetuximab and abemaciclib or a salt thereof are administered together in the same or separate compositions. In some embodiments, cobimetinib or a salt thereof and abemaciclib or a salt thereof are administered together in the same or separate compositions. In some embodiments, cetuximab, cobimetinib or a salt thereof and abemaciclib or a salt thereof are administered in the same composition. In some embodiments, cetuximab is administered every week or every two weeks, while cobimetinib or a salt thereof and abemaciclib or a salt thereof are administered on a daily basis.

In some embodiments, intermittent administrations are about 1-30 minutes apart, about 30-60 minutes apart, about 60-120 minutes apart, about 120-240 minutes apart, about 240-480 minutes apart, about 480-720 minutes apart, about 720-960 minutes apart or about 960-1440 minutes apart. In some embodiments, intermittent administrations are about 1-2 days apart, 2-3 days apart, 3-4 days apart, 4-5 days apart, 5-6 days apart, or 6-7 days apart.

In another aspect, the subject has been previously treated with a KRAS inhibitor. In another aspect, the subject has not been previously treated with a KRAS inhibitor. In another aspect, the subject has been previously treated with a combination of KRAS inhibitor and a second active agent. In another aspect, the subject has not been previously treated with a combination of KRAS inhibitor and a second active agent. In some embodiments, the subject has developed acquired or adaptive resistance to a KRAS inhibitor. KRAS inhibitors include, without limitation, a small molecule or antibody that specifically binds to wild or mutated KRAS or a ligand thereof, such as AMG-510 (“Amgen”; pyrido(2,3-d)pyrimidin-2(1H)-one, 6-fluoro-7-(2-fluoro-6-hydroxyphenyl)-1-(4-methyl-2-(1-methylethyl)-3-pyridinyl)-4-((2S)-1-oxo-2-propen-1-yl)-1-piperazinyl)), DCAI (2-(4,6-dichloro-2-methyl-1h-indol-3-yl)ethanamine,4,6-Dichloro-2-methyl-3-aminoethylindole), MRTX849 and ARS-1620.

In another aspect, the method described herein reduces cancer cell growth and/or increase cancer cell-killing by at least about 5%, 10%, 15%, 20%, 25%, 30%, 35%, 40%, 45%, 50%, 55%, 60%, 65%, 70%, 75%, 80%, 85%, 90%, 100%, 200%, 300%, 400%, 500%, 600%, 700%, 800%, 900%, 1000%, 2000% or more than administration of only one or two of (a) an epidermal growth factor receptor (EGFR) inhibitor (e.g., osimertinib, lapatinib, or cetuximab); (b) a mitogen-activated protein kinase (MEK) 1/2 inhibitor (e.g., cobimetinib, trametinib, binimetinib, or TAK-733); and (c) a cyclin dependent kinase (CDK) 4/6 inhibitor (e.g., palbociclib or abemaciclib). And the method described herein has demonstrated a synergistic effect upon treatment of cancer with a KRAS mutation. In some embodiments, the efficacy of the method described herein is at least about 5%, 10%, 15%, 20%, 25%, 30%, 35%, 40%, 45%, 50%, 55%, 60%, 65%, 70%, 75%, 80%, 85%, 90%, 100%, 200%, 300%, 400%, 500%, 600%, 700%, 800%, 900%, 1000%, 2000% or more than the additive efficacy of the individual administration of (a) an epidermal growth factor receptor (EGFR) inhibitor (e.g., osimertinib, lapatinib, or cetuximab); (b) a mitogen-activated protein kinase (MEK) 1/2 inhibitor (e.g., cobimetinib, trametinib, binimetinib, or TAK-733); or (c) a cyclin dependent kinase (CDK) 4/6 inhibitor (e.g., palbociclib or abemaciclib).

In another aspect, the method described herein reduces mean tumor volume by about 20-95%. In some embodiments, the mean tumor volume is reduced by at least about 10%, 15%, 20%, 25%, 30%, 35%, 40%, 45%, 50%, 55%, 60%, 65%, 70%, 75%, 80%, 85%, 90%, or 95%. In another aspect, the method described herein reduces mean tumor volume in a subject by about 20-95%. In some embodiments, the mean tumor volume in a subject is reduced by at least about 10%, 15%, 20%, 25%, 30%, 35%, 40%, 45%, 50%, 55%, 60%, 65%, 70%, 75%, 80%, 85%, 90%, or 95%.

In another aspect, the method described herein causes body weight changes in the subject at less than about 25%, less than about 20%, less than about 15%, or less than about 5%. In some embodiments, the method does not cause body weight change.

In another aspect, the method described herein has a Maximum Inhibition Index (MI) value of at least about 10, at least about 25, at least about 50, at least about 75, at least about 100, at least about 150, at least about 200, at least about 500, at least about 750, at least about 1000, at least about 2000, at least about 3000, at least about 4000, at least about 5000, or at least about 7500.

In yet another aspect, the method described herein comprises administration of a loading dose of the combination of (a) an epidermal growth factor receptor (EGFR) inhibitor (e.g., osimertinib, lapatinib, or cetuximab); (b) a mitogen-activated protein kinase (MEK) 1/2 inhibitor (e.g., cobimetinib, trametinib, binimetinib, or TAK-733); and (c) a cyclin dependent kinase (CDK) 4/6 inhibitor (e.g., palbociclib or abemaciclib) followed by multiple separate maintenance doses of the combination. In some embodiments, each loading dose of the three inhibitors is higher than each maintenance doses. In some embodiments, the method described herein provides lower dosages, safety and/or tolerability for long-term administrations and/or treatments.

### III. Compositions

Also provided herein are compositions comprising (a) an epidermal growth factor receptor (EGFR) inhibitor (e.g., osimertinib, lapatinib, or cetuximab); (b) a mitogen-activated protein kinase (MEK) 1/2 inhibitor (e.g., cobimetinib, trametinib, binimetinib, or TAK-733); and (c) a cyclin dependent kinase (CDK) 4/6 inhibitor (e.g., palbociclib or abemaciclib); wherein the composition does not comprises a KRAS inhibitor.

Also provided herein is a composition comprising osimertinib or a salt thereof, cobimetinib or a salt thereof, and palbociclib or a salt thereof. Also provided herein is a composition comprising cetuximab, cobimetinib or a salt thereof, and palbociclib or a salt thereof. Also provided herein is a composition comprising osimertinib or a salt thereof, TAK-733 or a salt thereof, and palbociclib or a salt thereof. Also provided herein is a composition comprising osimertinib or a salt thereof, trametinib or a salt thereof, and palbociclib or a salt thereof. Also provided herein is a composition comprising osimertinib or a salt thereof, TAK-733 or a salt thereof, and palbociclib or a salt thereof. Also provided herein is a composition comprising lapatinib or a salt thereof, trametinib or a salt thereof, and palbociclib or a salt thereof. Also provided herein is a composition comprising abemaciclib, cobimetinib or a salt thereof, and palbociclib or a salt thereof. Also provided herein is a composition comprising osimertinib or a salt thereof, binimetinib or a salt thereof, and palbociclib or a salt thereof. Also provided herein is a composition comprising cetuximab, binimetinib or a salt thereof, and palbociclib or a salt thereof. These compositions may be used for treating and delaying progression of cancer with a KRAS mutation in a method described herein. In some embodiments, the composition does not comprise a KRAS inhibitor.

In one aspect, the composition may further comprise a pharmaceutically acceptable carrier, excipient, binder, or diluent. A pharmaceutically-acceptable excipient is a substance that is non-toxic and otherwise biologically suitable for administration to a subject. Such excipients facilitate administration of the compounds described herein and are compatible with the active ingredient. Examples of pharmaceutically-acceptable excipients include stabilizers, lubricants, surfactants, diluents, anti-oxidants, binders, coloring agents, bulking agents, emulsifiers, or taste-modifying agents. In some embodiments, pharmaceutical compositions according to the embodiments are sterile compositions. Pharmaceutical compositions may be prepared using compounding techniques known or that become available to those skilled in the art. Sterile compositions are also contemplated by the embodiments, including compositions that are in accord with national and local regulations governing such compositions.

The pharmaceutical compositions and compounds described herein may be formulated as solutions, emulsions, suspensions, dispersions, or inclusion complexes such as cyclodextrins in suitable pharmaceutical solvents or carriers, or as pills, tablets, lozenges, suppositories, sachets, dragees, granules, powders, powders for reconstitution, or capsules along with solid carriers according to conventional methods known in the art for preparation of various dosage forms. Pharmaceutical compositions provided herein may be administered by a suitable route of delivery, such as oral, parenteral, rectal, nasal, or topical route, or by inhalation. In some embodiments, the compositions are formulated for intravenous or oral administration.

In another aspect, the composition is formulated for oral administration. For oral administration, composition may be formulated in a solid form, such as a tablet or capsule, or as a solution, emulsion, or suspension. Oral tablets may include the active ingredient(s) mixed with compatible pharmaceutically acceptable excipients such as diluents, disintegrating agents, binding agents, lubricating agents, sweetening agents, flavoring agents, coloring agents and preservative agents. Suitable inert fillers include sodium and calcium carbonate, sodium and calcium phosphate, lactose, starch, sugar, glucose, methyl cellulose, magnesium stearate, mannitol, sorbitol, and the like. Exemplary liquid oral excipients include ethanol, glycerol, water, and the like. Starch, polyvinyl-pyrrolidone (PVP), sodium starch glycolate, microcrystalline cellulose, and alginic acid are exemplary disintegrating agents. Binding agents may include starch and gelatin. The lubricating agent, if present, may be magnesium stearate, stearic acid, or talc. If desired, the tablets may be coated with a material such as glyceryl monostearate or glyceryl distearate to delay absorption in the gastrointestinal tract, or may be coated with an enteric coating.

Capsules for oral administration include hard and soft gelatin capsules. To prepare hard gelatin capsules, active ingredient(s) may be mixed with a solid, semi-solid, or liquid diluent. Soft gelatin capsules may be prepared by mixing the active ingredient with water, an oil such as peanut oil or olive oil, liquid paraffin, a mixture of mono and di-glycerides of short chain fatty acids, polyethylene glycol 400, or propylene glycol.

Liquids for oral administration may be in the form of suspensions, solutions, emulsions, or syrups, or may be lyophilized or presented as a dry product for reconstitution with water or other suitable vehicle before use. Such liquid compositions may optionally contain: pharmaceutically-acceptable excipients such as suspending agents (for example, sorbitol, methyl cellulose, sodium alginate, gelatin, hydroxyethylcellulose, carboxymethylcellulose, aluminum stearate gel and the like); non-aqueous vehicles, e.g., oil (for example, almond oil or fractionated coconut oil), propylene glycol, ethyl alcohol, or water; preservatives (for example, methyl or propyl p-hydroxybenzoate or sorbic acid); wetting agents such as lecithin; and, if desired, flavoring or coloring agents.

In another aspect, the composition comprises osimertinib or a salt thereof, cobimetinib or a salt thereof, and palbociclib or a salt thereof. In some embodiments, the composition comprises osimertinib or a salt thereof at greater than about 20%, or greater than about 25%, or greater than about 30%, or greater than about 35%, or greater than about 40%, or greater than about 45%, or greater than about 50%, or greater than about 55%, or greater than about 60%, or greater than about 65%, or greater than about 70% by weight.

In some embodiments, the composition comprises cobimetinib or a salt thereof at greater than about 20%, or greater than about 25%, or greater than about 30%, or greater than about 35%, or greater than about 40%, or greater than about 45%, or greater than about 50% by weight.

In some embodiments, the composition comprises palbociclib or a salt thereof at greater than about 30%, or greater than about 35%, or greater than about 40%, or greater than about 45%, or greater than about 50%, or greater than about 55%, or greater than about 60%, or greater than about 65%, or greater than about 70%, or greater than about 75%, or greater than about 80%, or greater than about 85%, or greater than about 90% by weight.

In some embodiments, the ratios of osimertinib or a salt thereof, cobimetinib or a salt thereof, and palbociclib or a salt thereof by weight in the compositions are about 1:1:1, 2:1:1, 3:1:1, 4:1:1, 1:2:1, 1:1:2, 1:1:3, 1:1:4, 1:1:5, 1:1:6, 1:1:7, 1:1:8, 1:1:9, 1:1:10, 2:1:2, 2:1:3, 2:1:4, 2:1:5, 2:1:6, 2:1:7, 2:1:8, 2:1:9, 2:1:10, 3:1:1, 3:1:2, 3:1:3, 3:1:4, 3:1:5, 3:1:6, 3:1:7, 3:1:8, 3:1:9, or 3:1:10. In some embodiments, the ratio of osimertinib or a salt thereof, cobimetinib or a salt thereof, and palbociclib or a salt thereof by weight in the compositions is about 2:1:4. In some embodiments, the ratio of osimertinib or a salt thereof and cobimetinib or a salt thereof by weight is in the range of about 2:3 to 4:1. In some embodiments, the ratio of osimertinib or a salt thereof and palbociclib or a salt thereof by weight is in the range of about 40:125 to 80:75. In some embodiments, the ratio of cobimetinib or a salt thereof and palbociclib or a salt thereof by weight is in the range of about 20:125 to 60:75.

In another aspect, the composition comprises osimertinib or a salt thereof, TAK-733 or a salt thereof, and palbociclib or a salt thereof. In some embodiments, the composition comprises osimertinib or a salt thereof at greater than about 20%, or greater than about 25%, or greater than about 30%, or greater than about 35%, or greater than about 40%, or greater than about 45%, or greater than about 50%, or greater than about 55%, or greater than about 60%, or greater than about 65%, or greater than about 70% by weight.

In some embodiments, the composition comprises TAK-733 or a salt thereof at greater than about 20%, or greater than about 25%, or greater than about 30%, or greater than about 35%, or greater than about 40%, or greater than about 45%, or greater than about 50% by weight.

In some embodiments, the composition comprises palbociclib or a salt thereof at greater than about 30%, or greater than about 35%, or greater than about 40%, or greater than about 45%, or greater than about 50%, or greater than about 55%, or greater than about 60%, or greater than about 65%, or greater than about 70%, or greater than about 75%, or greater than about 80%, or greater than about 85%, or greater than about 90% by weight.

In some embodiments, the ratios of osimertinib or a salt thereof, TAK-733 or a salt thereof and palbociclib or a salt thereof by weight in the compositions are about 1:1:1, 2:1:1, 3:1:1, 4:1:1, 1:2:1, 1:1:2, 1:1:3, 1:1:4, 1:1:5, 1:1:6, 1:1:7, 1:1:8, 1:1:9, 1:1:10, 2:1:2, 2:1:3, 2:1:4, 2:1:5, 2:1:6, 2:1:7, 2:1:8, 2:1:9, 2:1:10, 3:1:1, 3:1:2, 3:1:3, 3:1:4, 3:1:5, 3:1:6, 3:1:7, 3:1:8, 3:1:9, or 3:1:10. In some embodiments, the ratio of osimertinib or a salt thereof and TAK-733 or a salt thereof by weight in the compositions is in the range of about 5:2 to 10:1. In some embodiments, the ratio of osimertinib or a salt thereof and palbociclib or a salt thereof by weight in the compositions is in the range of about 40:125 to 80:75. In some embodiments, the ratio of TAK-733 or a salt thereof and palbociclib or a salt thereof by weight in the compositions is in the range of about 8:125 to 16:75.

In another aspect, the composition comprises lapatinib or a salt thereof, trametinib or a salt thereof, and palbociclib or a salt thereof. In some embodiments, the composition comprises lapatinib or a salt thereof at greater than about 20%, or greater than about 25%, or greater than about 30%, or greater than about 35%, or greater than about 40%, or greater than about 45%, or greater than about 50%, or greater than about 55%, or greater than about 60%, or greater than about 65%, or greater than about 70%, or greater than about 75%, or greater than about 80%, or greater than about 85%, or greater than about 90% by weight.

In some embodiments, the composition comprises trametinib or a salt thereof at greater than about 20%, or greater than about 25%, or greater than about 30%, or greater than about 35%, or greater than about 40%, or greater than about 45%, or greater than about 50% by weight.

In some embodiments, the composition comprises palbociclib or a salt thereof at greater than about 30%, or greater than about 35%, or greater than about 40%, or greater than about 45%, or greater than about 50%, or greater than about 55%, or greater than about 60%, or greater than about 65%, or greater than about 70%, or greater than about 75%, or greater than about 80%, or greater than about 85%, or greater than about 90% by weight.

In some embodiments, the ratios of lapatinib or a salt thereof, trametinib or a salt thereof, and palbociclib or a salt thereof by weight in the compositions are about 1:1:1, 2:1:1, 3:1:1, 4:1:1, 1:2:1, 1:1:2, 1:1:3, 1:1:4, 1:1:5, 1:1:6, 1:1:7, 1:1:8, 1:1:9, 1:1:10, 2:1:2, 2:1:3, 2:1:4, 2:1:5, 2:1:6, 2:1:7, 2:1:8, 2:1:9, 2:1:10, 3:1:1, 3:1:2, 3:1:3, 3:1:4, 3:1:5, 3:1:6, 3:1:7, 3:1:8, 3:1:9, 3:1:10, 4:1:3, 5:1:3, 6:1:3, 7:1:3, 8:1:3, 9:1:3, 10:1:3, 20:1:3, or 30:1:3. In some embodiments, the ratio of lapatinib or a salt thereof, trametinib or a salt thereof, and palbociclib or a salt thereof by weight in the compositions is about 20:1:3. In some embodiments, the ratio of lapatinib or a salt thereof and trametinib or a salt thereof by weight is in the range of about 2:3 to 40:1. In some embodiments, the ratio of lapatinib or a salt thereof and palbociclib or a salt thereof by weight is in the range of about 2:3 to 20:1. In some embodiments, the ratio of trametinib or a salt thereof and palbociclib or a salt thereof by weight is in the range of about 20:125 to 60:75.

In another aspect, the composition comprises osimertinib or a salt thereof, binimetinib or a salt thereof, and palbociclib or a salt thereof. In some embodiments, the composition comprises osimertinib or a salt thereof at greater than about 20%, or greater than about 25%, or greater than about 30%, or greater than about 35%, or greater than about 40%, or greater than about 45%, or greater than about 50%, or greater than about 55%, or greater than about 60%, or greater than about 65%, or greater than about 70% by weight.

In some embodiments, the composition comprises binimetinib or a salt thereof at greater than about 20%, or greater than about 25%, or greater than about 30%, or greater than about 35%, or greater than about 40%, or greater than about 45%, or greater than about 50% by weight.

In some embodiments, the composition comprises palbociclib or a salt thereof at greater than about 30%, or greater than about 35%, or greater than about 40%, or greater than about 45%, or greater than about 50%, or greater than about 55%, or greater than about 60%, or greater than about 65%, or greater than about 70%, or greater than about 75%, or greater than about 80%, or greater than about 85%, or greater than about 90% by weight.

In some embodiments, the ratios of osimertinib or a salt thereof, binimetinib or a salt thereof, and palbociclib or a salt thereof by weight in the compositions are about 1:1:1, 2:1:1, 3:1:1, 4:1:1, 1:2:1, 1:1:2, 1:1:3, 1:1:4, 1:1:5, 1:1:6, 1:1:7, 1:1:8, 1:1:9, 1:1:10, 2:1:2, 2:1:3, 2:1:4, 2:1:5, 2:1:6, 2:1:7, 2:1:8, 2:1:9, 2:1:10, 3:1:1, 3:1:2, 3:1:3, 3:1:4, 3:1:5, 3:1:6, 3:1:7, 3:1:8, 3:1:9, or 3:1:10. In some embodiments, the ratio of osimertinib or a salt thereof, binimetinib or a salt thereof, and palbociclib or a salt thereof by weight in the compositions is about 2:1:4. In some embodiments, the ratio of osimertinib or a salt thereof and binimetinib or a salt thereof by weight is in the range of about 2:3 to 4:1. In some embodiments, the ratio of osimertinib or a salt thereof and palbociclib or a salt thereof by weight is in the range of about 40:125 to 80:75. In some embodiments, the ratio of binimetinib or a salt thereof and palbociclib or a salt thereof by weight is in the range of about 20:125 to 60:75.

In another aspect, the composition comprises salts of osimertinib, lapatinib, cobimetinib, TAK-733 or palbociclib. In some embodiments, the salts are pharmaceutically acceptable salts. Non-limiting examples of pharmaceutically acceptable salts include, without limitation, sulfates, pyrosulfates, bisulfates, sulfites, bisulfites, phosphates, monohydrogen-phosphates, dihydrogenphosphates, metaphosphates, pyrophosphates, chlorides, bromides, iodides, acetates, propionates, decanoates, caprylates, acrylates, formates, isobutyrates, caproates, heptanoates, propiolates, oxalates, malonates, succinates, suberates, sebacates, fumarates, maleates, butyne-1,4-dioates, hexyne-1,6-dioates, benzoates, chlorobenzoates, methylbenzoates, dinitrobenzoates, hydroxybenzoates, methoxybenzoates, phthalates, sulfonates, methylsulfonates, propylsulfonates, besylates, xylenesulfonates, naphthalene-1-sulfonates, naphthalene-2-sulfonates, phenylacetates, mesylates phenylpropionates, phenylbutyrates, citrates, lactates, γ-hydroxybutyrates, glycolates, tartrates, and mandelates. In some embodiments, the composition comprises mesylate salts of osimertinib, cobimetinib, TAK-733 or palbociclib. In some embodiments, the composition comprises fumarate salts of osimertinib, cobimetinib, TAK-733 or palbociclib. In some embodiments, the composition comprises a solvate of osimertinib, cobimetinib, TAK-733 or palbociclib.

### IV. Kits

Also provided herein are kits comprising (a) an epidermal growth factor receptor (EGFR) inhibitor (e.g., osimertinib, lapatinib, or cetuximab); (b) a mitogen-activated protein kinase (MEK) 1/2 inhibitor (e.g., cobimetinib, trametinib, binimetinib, or TAK-733); and (c) a cyclin dependent kinase (CDK) 4/6 inhibitor (e.g., palbociclib or abemaciclib); wherein the kit does not comprises a KRAS inhibitor. The kits may be used for treating and delaying progression of cancer (e.g., colorectal cancer) with a KRAS mutation in a method described herein. The kit may comprise any compositions described herein.

Also provided herein is a kit comprising osimertinib or a salt thereof, cobimetinib or a salt thereof, and palbociclib or a salt thereof. In some embodiments, the kit comprises a pharmaceutical composition comprising osimertinib or a salt thereof and a pharmaceutically acceptable carrier, excipient, binder, or diluent. In some embodiments, the kit comprises a pharmaceutical composition comprising cobimetinib or a salt thereof and a pharmaceutically acceptable carrier, excipient, binder, or diluent. In some embodiments, the kit comprises a pharmaceutical composition comprising palbociclib or a salt thereof and a pharmaceutically acceptable carrier, excipient, binder, or diluent.

In some embodiments, osimertinib or a salt thereof, cobimetinib or a salt thereof, and palbociclib or a salt thereof are formulated as one composition in the kit. In some embodiments, osimertinib or a salt thereof, cobimetinib or a salt thereof, and palbociclib or a salt thereof are formulated separately. In some embodiments, osimertinib or a salt thereof, cobimetinib or a salt thereof, and/or palbociclib or a salt thereof are formulated for oral administration. In some embodiments, osimertinib or a salt thereof, cobimetinib or a salt thereof, and palbociclib or a salt thereof are formulated in the same form, such as solid or liquid form. In some embodiments, osimertinib or a salt thereof, cobimetinib or a salt thereof, and/or palbociclib or a salt thereof are formulated as solutions, emulsions, suspensions, dispersions, or inclusion complexes such as cyclodextrins in suitable pharmaceutical solvents or carriers, or as pills, tablets, lozenges, suppositories, sachets, dragees, granules, powders, powders for reconstitution, or capsules along with solid carriers according to conventional methods known in the art for preparation of various dosage forms.

In some embodiments, the ratios of osimertinib or a salt thereof, cobimetinib or a salt thereof, and palbociclib or a salt thereof by weight provided in the kits are about 1:1:1:, 2:1:1, 3:1:1, 4:1:1:, 1:2:1, 1:1:2, 1:1:3, 1:1:4, 1:1:5, 1:1:6, 1:1:7, 1:1:8, 1:1:9, 1:1:10, 2:1:2, 2:1:3, 2:1:4, 2:1:5, 2:1:6, 2:1:7, 2:1:8, 2:1:9, 2:1:10, 3:1:1, 3:1:2, 3:1:3, 3:1:4, 3:1:5, 3:1:6, 3:1:7, 3:1:8, 3:1:9, or 3:1:10. In some embodiments, the ratio of osimertinib or a salt thereof, cobimetinib or a salt thereof, and palbociclib or a salt thereof by weight in the kits is about 2:1:4. In some embodiments, the ratio of osimertinib or a salt thereof and cobimetinib or a salt thereof by weight in the kits is in the range of about 2:3 to 4:1. In some embodiments, the ratio of osimertinib or a salt thereof and palbociclib or a salt thereof by weight in the kits is in the range of about 40:125 to 80:75. In some embodiments, the ratio of cobimetinib or a salt thereof and palbociclib or a salt thereof by weight in the kits is in the range of about 20:125 to 60:75.

Also provided herein is a kit comprising cetuximab, cobimetinib or a salt thereof, and palbociclib or a salt thereof. In some embodiments, the kit comprises a pharmaceutical composition comprising cetuximab and a pharmaceutically acceptable carrier, excipient, binder, or diluent. In some embodiments, the kit comprises a pharmaceutical composition comprising cobimetinib or a salt thereof and a pharmaceutically acceptable carrier, excipient, binder, or diluent. In some embodiments, the kit comprises a pharmaceutical composition comprising palbociclib or a salt thereof and a pharmaceutically acceptable carrier, excipient, binder, or diluent.

In some embodiments, cetuximab, cobimetinib or a salt thereof, and palbociclib or a salt thereof are formulated as one composition in the kit. In some embodiments, cetuximab, cobimetinib or a salt thereof, and palbociclib or a salt thereof are formulated as two or more compositions in the kit. In some embodiments, cetuximab, cobimetinib or a salt thereof, and palbociclib or a salt thereof are formulated separately. In some embodiments, cobimetinib or a salt thereof, and/or palbociclib or a salt thereof are formulated for oral administration. In some embodiments, cobimetinib or a salt thereof, and palbociclib or a salt thereof are formulated in the same form, such as solid or liquid form. In some embodiments, cetuximab, cobimetinib or a salt thereof, and/or palbociclib or a salt thereof are formulated as solutions, emulsions, suspensions, dispersions, or inclusion complexes such as cyclodextrins in suitable pharmaceutical solvents or carriers, or as pills, tablets, lozenges, suppositories, sachets, dragees, granules, powders, powders for reconstitution, or capsules along with solid carriers according to conventional methods known in the art for preparation of various dosage forms. In some embodiments, cetuximab is formulated for administration via intravenous infusion. In some embodiments, cobimetinib or a salt thereof and palbociclib or a salt thereof are formulated as one composition for oral administration.

In some embodiments, the ratios of cetuximab, cobimetinib or a salt thereof, and palbociclib or a salt thereof by weight provided in the kits are about 1:1:1:, 2:1:1, 3:1:1, 4:1:, 1:2:1, 3:1:2, 3:1:3, 3:1:4, 3:1:5, 3:1:6, 3:1:7, 3:1:8, 3:1:9, 3:1:10, 4:1:2, 4:1:3, 4:1:4, 4:1:5, 4:1:6, 4:1:7, 4:1:8, 4:1:9, 4:1:10, 5:1:1, 5:1:2, 5:1:3, 5:1:4, 5:1:5, 5:1:6, 5:1:7, 5:1:8, 5:1:9, 5:1:10, 6:1:1, 6:1:2, 6:1:3, 6:1:4, 6:1:5, 6:1:6, 6:1:7, 6:1:8, 6:1:9, or 6:1:10. In some embodiments, the ratio of cetuximab, cobimetinib or a salt thereof, and palbociclib or a salt thereof by weight in the kits is about 20:3:6. In some embodiments, the ratio of cetuximab and cobimetinib or a salt thereof by weight in the kits is the range of about 85:60 to 85:2. In some embodiments, the ratio of cetuximab and palbociclib or a salt thereof by weight in the kits is the range of about 17:25 to 34:3. In some embodiments, the ratio of cobimetinib or a salt thereof and palbociclib or a salt thereof by weight in the kits is the range of about 20:125 to 60:75.

In some embodiments, cetuximab, TAK-733 or a salt thereof, and palbociclib or a salt thereof are formulated as one composition in the kit. In some embodiments, cetuximab, TAK-733 or a salt thereof and palbociclib or a salt thereof are formulated as two or more compositions in the kit. In some embodiments, cetuximab, TAK-733 or a salt thereof and palbociclib or a salt thereof are formulated separately. In some embodiments, TAK-733 or a salt thereof and/or palbociclib or a salt thereof are formulated for oral administration. In some embodiments, TAK-733 or a salt thereof and palbociclib or a salt thereof are formulated in the same form, such as solid or liquid form. In some embodiments, cetuximab, TAK-733 or a salt thereof and/or palbociclib or a salt thereof are formulated as solutions, emulsions, suspensions, dispersions, or inclusion complexes such as cyclodextrins in suitable pharmaceutical solvents or carriers, or as pills, tablets, lozenges, suppositories, sachets, dragees, granules, powders, powders for reconstitution, or capsules along with solid carriers according to conventional methods known in the art for preparation of various dosage forms. In some embodiments, cetuximab is formulated for administration via intravenous infusion. In some embodiments, TAK-733 or a salt thereof and palbociclib or a salt thereof are formulated as one composition for oral administration.

In some embodiments, the ratios of cetuximab, TAK-733 or a salt thereof and palbociclib or a salt thereof by weight provided in the kits are about 5:1:5:, 10:1:5, 15:1:5, 20:1:5:, 10:2:1, 10:1:2, 10:1:3, 10:1:4, 10:1:5, 10:1:6, 10:1:7, 10:1:8, 5:1:9, 10:1:10, 15:1:2, 15:1:3, 15:1:4, 15:1:5, 15:1:6, 15:1:7, 15:1:8, 15:1:9, 15:1:10, 20:1:1, 20:1:2, 20:1:3, 20:1:4, 20:1:5, 20:1:6, 20:1:7, 20:1:8, 20:1:9, or 20:1:10. In some embodiments, the ratio of cetuximab, TAK-733 or a salt thereof and palbociclib or a salt thereof by weight in the kits is about 20:1:6. In some embodiments, the ratio of cetuximab and TAK-733 or a salt thereof by weight in the kits is in the range of about 85:16 to 850:8. In some embodiments, the ratio of cetuximab and palbociclib or a salt thereof by weight in the kits is in the range of about 17:25 to 34:3. In some embodiments, the ratio of TAK-733 or a salt thereof and palbociclib or a salt thereof by weight in the kits is in the range of about 8:125 to 16:75.

Also provided herein is a kit comprising osimertinib or a salt thereof, TAK-733 or a salt thereof, and palbociclib or a salt thereof. In some embodiments, the kit comprises a pharmaceutical composition comprising osimertinib or a salt thereof and a pharmaceutically acceptable carrier, excipient, binder, or diluent. In some embodiments, the kit comprises a pharmaceutical composition comprising TAK-733 or a salt thereof and a pharmaceutically acceptable carrier, excipient, binder, or diluent. In some embodiments, the kit comprises a pharmaceutical composition comprising palbociclib or a salt thereof and a pharmaceutically acceptable carrier, excipient, binder, or diluent.

In some embodiments, osimertinib or a salt thereof, TAK-733 or a salt thereof, and palbociclib or a salt thereof are formulated as one composition in the kit. In some embodiments, osimertinib or a salt thereof, TAK-733 or a salt thereof, and palbociclib or a salt thereof are formulated separately. In some embodiments, osimertinib or a salt thereof, TAK-733 or a salt thereof, and/or palbociclib or a salt thereof are formulated for oral administration. In some embodiments, osimertinib or a salt thereof, TAK-733 or a salt thereof, and palbociclib or a salt thereof are formulated in the same form, such as solid or liquid form. In some embodiments, osimertinib or a salt thereof, TAK-733 or a salt thereof, and/or palbociclib or a salt thereof are formulated as solutions, emulsions, suspensions, dispersions, or inclusion complexes such as cyclodextrins in suitable pharmaceutical solvents or carriers, or as pills, tablets, lozenges, suppositories, sachets, dragees, granules, powders, powders for reconstitution, or capsules along with solid carriers according to conventional methods known in the art for preparation of various dosage forms.

In some embodiments, the ratios of osimertinib or a salt thereof, TAK-733 or a salt thereof, and palbociclib or a salt thereof by weight provided in the kits are about 1:1:1:, 2:1:1, 3:1:1, 4:1:1:, 1:2:1, 1:1:2, 1:1:3, 1:1:4, 1:1:5, 1:1:6, 1:1:7, 1:1:8, 1:1:9, 1:1:10, 2:1:2, 2:1:3, 2:1:4, 2:1:5, 2:1:6, 2:1:7, 2:1:8, 2:1:9, 2:1:10, 3:1:1, 3:1:2, 3:1:3, 3:1:4, 3:1:5, 3:1:6, 3:1:7, 3:1:8, 3:1:9, or 3:1:10. In some embodiments, the ratio of osimertinib or a salt thereof, TAK-733 or a salt thereof and palbociclib or a salt thereof by weight in the compositions is about 3:1:6. In some embodiments, the ratio of osimertinib or a salt thereof and TAK-733 or a salt thereof by weight in the kits is in the range of about 5:2 to 10:1. In some embodiments, the ratio of osimertinib or a salt thereof and palbociclib or a salt thereof by weight in the kits is in the range of about 40:125 to 80:75. In some embodiments, the ratio of TAK-733 or a salt thereof and palbociclib or a salt thereof by weight in the kits is in the range of about 8:125 to 16:75.

Also provided herein is a kit comprising cetuximab, trametinib or a salt thereof, and palbociclib or a salt thereof. In some embodiments, the kit comprises a pharmaceutical composition comprising cetuximab and a pharmaceutically acceptable carrier, excipient, binder, or diluent. In some embodiments, the kit comprises a pharmaceutical composition comprising trametinib or a salt thereof and a pharmaceutically acceptable carrier, excipient, binder, or diluent. In some embodiments, the kit comprises a pharmaceutical composition comprising palbociclib or a salt thereof and a pharmaceutically acceptable carrier, excipient, binder, or diluent.

In some embodiments, cetuximab, trametinib or a salt thereof and palbociclib or a salt thereof are formulated as two or more compositions (e.g., two or three compositions) in the kit. In some embodiments, cetuximab, trametinib or a salt thereof and palbociclib or a salt thereof are formulated separately. In some embodiments, trametinib or a salt thereof and/or palbociclib or a salt thereof are formulated for oral administration. In some embodiments, trametinib or a salt thereof and palbociclib or a salt thereof are formulated in the same form, such as solid or liquid form. In some embodiments, cetuximab, trametinib or a salt thereof and/or palbociclib or a salt thereof are formulated as solutions, emulsions, suspensions, dispersions, or inclusion complexes such as cyclodextrins in suitable pharmaceutical solvents or carriers, or as pills, tablets, lozenges, suppositories, sachets, dragees, granules, powders, powders for reconstitution, or capsules along with solid carriers according to conventional methods known in the art for preparation of various dosage forms. In some embodiments, cetuximab is formulated for administration via intravenous infusion. In some embodiments, trametinib or a salt thereof and palbociclib or a salt thereof are formulated as one composition for oral administration.

Also provided herein is a kit comprising cetuximab, binimetinib or a salt thereof, and palbociclib or a salt thereof. In some embodiments, the kit comprises a pharmaceutical composition comprising cetuximab and a pharmaceutically acceptable carrier, excipient, binder, or diluent. In some embodiments, the kit comprises a pharmaceutical composition comprising binimetinib or a salt thereof, and a pharmaceutically acceptable carrier, excipient, binder, or diluent. In some embodiments, the kit comprises a pharmaceutical composition comprising palbociclib or a salt thereof and a pharmaceutically acceptable carrier, excipient, binder, or diluent.

In some embodiments, cetuximab, binimetinib or a salt thereof, and palbociclib or a salt thereof are formulated as two or more compositions in the kit. In some embodiments, cetuximab, binimetinib or a salt thereof, and palbociclib or a salt thereof are formulated separately. In some embodiments, binimetinib or a salt thereof, and/or palbociclib or a salt thereof are formulated for oral administration. In some embodiments, binimetinib or a salt thereof, and palbociclib or a salt thereof are formulated in the same form, such as solid or liquid form. In some embodiments, cetuximab, binimetinib or a salt thereof, and/or palbociclib or a salt thereof are formulated as solutions, emulsions, suspensions, dispersions, or inclusion complexes such as cyclodextrins in suitable pharmaceutical solvents or carriers, or as pills, tablets, lozenges, suppositories, sachets, dragees, granules, powders, powders for reconstitution, or capsules along with solid carriers according to conventional methods known in the art for preparation of various dosage forms. In some embodiments, cetuximab is formulated for administration via intravenous infusion. In some embodiments, binimetinib or a salt thereof and palbociclib or a salt thereof are formulated as one composition for oral administration.

In some embodiments, the ratios of cetuximab, binimetinib or a salt thereof, and palbociclib or a salt thereof by weight provided in the kits are about 1:1:1:, 2:1:1, 3:1:1, 4:1:1:, 1:2:1, 3:1:2, 3:1:3, 3:1:4, 3:1:5, 3:1:6, 3:1:7, 3:1:8, 3:1:9, 3:1:10, 4:1:2, 4:1:3, 4:1:4, 4:1:5, 4:1:6, 4:1:7, 4:1:8, 4:1:9, 4:1:10, 5:1:1, 5:1:2, 5:1:3, 5:1:4, 5:1:5, 5:1:6, 5:1:7, 5:1:8, 5:1:9, 5:1:10, 6:1:1, 6:1:2, 6:1:3, 6:1:4, 6:1:5, 6:1:6, 6:1:7, 6:1:8, 6:1:9, or 6:1:10. In some embodiments, the ratio of cetuximab, binimetinib or a salt thereof, and palbociclib or a salt thereof by weight in the kits is about 20:3:6. In some embodiments, the ratio of cetuximab and binimetinib or a salt thereof by weight in the kits is the range of about 85:60 to 85:2. In some embodiments, the ratio of cetuximab and palbociclib or a salt thereof by weight in the kits is the range of about 17:25 to 34:3. In some embodiments, the ratio of binimetinib or a salt thereof and palbociclib or a salt thereof by weight in the kits is the range of about 20:125 to 60:75.

Also provided herein is a kit comprising osimertinib or a salt thereof, binimetinib or a salt thereof, and palbociclib or a salt thereof. In some embodiments, the kit comprises a pharmaceutical composition comprising osimertinib or a salt thereof and a pharmaceutically acceptable carrier, excipient, binder, or diluent. In some embodiments, the kit comprises a pharmaceutical composition comprising binimetinib or a salt thereof and a pharmaceutically acceptable carrier, excipient, binder, or diluent. In some embodiments, the kit comprises a pharmaceutical composition comprising palbociclib or a salt thereof and a pharmaceutically acceptable carrier, excipient, binder, or diluent.

In some embodiments, osimertinib or a salt thereof, binimetinib or a salt thereof, and palbociclib or a salt thereof are formulated as one composition in the kit. In some embodiments, osimertinib or a salt thereof, binimetinib or a salt thereof, and palbociclib or a salt thereof are formulated as two or more compositions in the kit. In some embodiments, osimertinib or a salt thereof, binimetinib or a salt thereof, and palbociclib or a salt thereof are formulated separately. In some embodiments, osimertinib or a salt thereof, binimetinib or a salt thereof, and/or palbociclib or a salt thereof are formulated for oral administration. In some embodiments, osimertinib or a salt thereof, binimetinib or a salt thereof, and palbociclib or a salt thereof are formulated in the same form, such as solid or liquid form. In some embodiments, osimertinib or a salt thereof, binimetinib or a salt thereof, and/or palbociclib or a salt thereof are formulated as solutions, emulsions, suspensions, dispersions, or inclusion complexes such as cyclodextrins in suitable pharmaceutical solvents or carriers, or as pills, tablets, lozenges, suppositories, sachets, dragees, granules, powders, powders for reconstitution, or capsules along with solid carriers according to conventional methods known in the art for preparation of various dosage forms. In some embodiments, osimertinib or a salt thereof, binimetinib or a salt thereof and palbociclib or a salt thereof are formulated as one composition for oral administration.

In some embodiments, the ratios of osimertinib or a salt thereof, binimetinib or a salt thereof, and palbociclib or a salt thereof by weight provided in the kits are about 1:1:1:, 2:1:1, 3:1:1, 4:1:1:, 1:2:1, 3:1:2, 3:1:3, 3:1:4, 3:1:5, 3:1:6, 3:1:7, 3:1:8, 3:1:9, 3:1:10, 4:1:2, 4:1:3, 4:1:4, 4:1:5, 4:1:6, 4:1:7, 4:1:8, 4:1:9, 4:1:10, 5:1:1, 5:1:2, 5:1:3, 5:1:4, 5:1:5, 5:1:6, 5:1:7, 5:1:8, 5:1:9, 5:1:10, 6:1:1, 6:1:2, 6:1:3, 6:1:4, 6:1:5, 6:1:6, 6:1:7, 6:1:8, 6:1:9, or 6:1:10. In some embodiments, the ratio of osimertinib or a salt thereof, binimetinib or a salt thereof, and palbociclib or a salt thereof by weight in the kits is about 20:3:6. In some embodiments, the ratio of osimertinib or a salt thereof and binimetinib or a salt thereof by weight in the kits is the range of about 85:60 to 85:2. In some embodiments, the ratio of osimertinib or a salt thereof and palbociclib or a salt thereof by weight in the kits is the range of about 17:25 to 34:3. In some embodiments, the ratio of binimetinib or a salt thereof and palbociclib or a salt thereof by weight in the kits is the range of about 20:125 to 60:75.

Also provided herein is a kit comprising cetuximab, cobimetinib or a salt thereof, and abemaciclib or a salt thereof. In some embodiments, the kit comprises a pharmaceutical composition comprising cetuximab and a pharmaceutically acceptable carrier, excipient, binder, or diluent. In some embodiments, the kit comprises a pharmaceutical composition comprising cobimetinib or a salt thereof and a pharmaceutically acceptable carrier, excipient, binder, or diluent. In some embodiments, the kit comprises a pharmaceutical composition comprising abemaciclib or a salt thereof and a pharmaceutically acceptable carrier, excipient, binder, or diluent.

In some embodiments, cetuximab, cobimetinib or a salt thereof, and abemaciclib or a salt thereof are formulated as one composition in the kit. In some embodiments, cetuximab, cobimetinib or a salt thereof, and abemaciclib or a salt thereof are formulated as two or more compositions in the kit. In some embodiments, cetuximab, cobimetinib or a salt thereof, and abemaciclib or a salt thereof are formulated separately. In some embodiments, cobimetinib or a salt thereof, and/or abemaciclib or a salt thereof are formulated for oral administration. In some embodiments, cobimetinib or a salt thereof, and abemaciclib or a salt thereof are formulated in the same form, such as solid or liquid form. In some embodiments, cetuximab, cobimetinib or a salt thereof, and/or abemaciclib or a salt thereof are formulated as solutions, emulsions, suspensions, dispersions, or inclusion complexes such as cyclodextrins in suitable pharmaceutical solvents or carriers, or as pills, tablets, lozenges, suppositories, sachets, dragees, granules, powders, powders for reconstitution, or capsules along with solid carriers according to conventional methods known in the art for preparation of various dosage forms. In some embodiments, cetuximab is formulated for administration via intravenous infusion. In some embodiments, cobimetinib or a salt thereof and abemaciclib or a salt thereof are formulated as one composition for oral administration.

In some embodiments, the ratios of cetuximab, cobimetinib or a salt thereof, and abemaciclib or a salt thereof by weight provided in the kits are about 1:1:1:, 2:1:1, 3:1:1, 4:1:1:, 1:2:1, 3:1:2, 3:1:3, 3:1:4, 3:1:5, 3:1:6, 3:1:7, 3:1:8, 3:1:9, 3:1:10, 4:1:2, 4:1:3, 4:1:4, 4:1:5, 4:1:6, 4:1:7, 4:1:8, 4:1:9, 4:1:10, 5:1:1, 5:1:2, 5:1:3, 5:1:4, 5:1:5, 5:1:6, 5:1:7, 5:1:8, 5:1:9, 5:1:10, 6:1:1, 6:1:2, 6:1:3, 6:1:4, 6:1:5, 6:1:6, 6:1:7, 6:1:8, 6:1:9, or 6:1:10. In some embodiments, the ratio of cetuximab, cobimetinib or a salt thereof, and abemaciclib or a salt thereof by weight in the kits is about 20:3:6. In some embodiments, the ratio of cetuximab and cobimetinib or a salt thereof by weight in the kits is the range of about 85:60 to 85:2. In some embodiments, the ratio of cetuximab and abemaciclib or a salt thereof by weight in the kits is the range of about 17:25 to 34:3. In some embodiments, the ratio of cobimetinib or a salt thereof and abemaciclib or a salt thereof by weight in the kits is the range of about 20:125 to 60:75.

In some embodiments, cetuximab, trametinib or a salt thereof, and palbociclib or a salt thereof are formulated as one composition in the kit. In some embodiments, cetuximab, trametinib or a salt thereof and palbociclib or a salt thereof are formulated as two or more compositions in the kit. In some embodiments, cetuximab, trametinib or a salt thereof and palbociclib or a salt thereof are formulated separately. In some embodiments, trametinib or a salt thereof and/or palbociclib or a salt thereof are formulated for oral administration. In some embodiments, trametinib or a salt thereof and palbociclib or a salt thereof are formulated in the same form, such as solid or liquid form. In some embodiments, cetuximab, trametinib or a salt thereof and/or palbociclib or a salt thereof are formulated as solutions, emulsions, suspensions, dispersions, or inclusion complexes such as cyclodextrins in suitable pharmaceutical solvents or carriers, or as pills, tablets, lozenges, suppositories, sachets, dragees, granules, powders, powders for reconstitution, or capsules along with solid carriers according to conventional methods known in the art for preparation of various dosage forms. In some embodiments, cetuximab is formulated for administration via intravenous infusion. In some embodiments, trametinib or a salt thereof and palbociclib or a salt thereof are formulated as one composition for oral administration.

Also provided herein is a kit comprising cetuximab, binimetinib or a salt thereof, and palbociclib or a salt thereof. In some embodiments, the kit comprises a pharmaceutical composition comprising cetuximab and a pharmaceutically acceptable carrier, excipient, binder, or diluent. In some embodiments, the kit comprises a pharmaceutical composition comprising binimetinib or a salt thereof and a pharmaceutically acceptable carrier, excipient, binder, or diluent. In some embodiments, the kit comprises a pharmaceutical composition comprising palbociclib or a salt thereof and a pharmaceutically acceptable carrier, excipient, binder, or diluent.

In some embodiments, cetuximab, binimetinib or a salt thereof, and palbociclib or a salt thereof are formulated as one composition in the kit. In some embodiments, cetuximab, binimetinib or a salt thereof, and palbociclib or a salt thereof are formulated as two or more compositions in the kit. In some embodiments, cetuximab, binimetinib or a salt thereof, and palbociclib or a salt thereof are formulated separately. In some embodiments, binimetinib or a salt thereof, and/or palbociclib or a salt thereof are formulated for oral administration. In some embodiments, binimetinib or a salt thereof, and palbociclib or a salt thereof are formulated in the same form, such as solid or liquid form. In some embodiments, cetuximab, binimetinib or a salt thereof, and/or palbociclib or a salt thereof are formulated as solutions, emulsions, suspensions, dispersions, or inclusion complexes such as cyclodextrins in suitable pharmaceutical solvents or carriers, or as pills, tablets, lozenges, suppositories, sachets, dragees, granules, powders, powders for reconstitution, or capsules along with solid carriers according to conventional methods known in the art for preparation of various dosage forms. In some embodiments, cetuximab is formulated for administration via intravenous infusion. In some embodiments, binimetinib or a salt thereof and palbociclib or a salt thereof are formulated as one composition for oral administration.

In some embodiments, the ratios of cetuximab, binimetinib or a salt thereof, and palbociclib or a salt thereof by weight provided in the kits are about 1:1:1:, 2:1:1, 3:1:1, 4:1:, 1:2:1, 3:1:2, 3:1:3, 3:1:4, 3:1:5, 3:1:6, 3:1:7, 3:1:8, 3:1:9, 3:1:10, 4:1:2, 4:1:3, 4:1:4, 4:1:5, 4:1:6, 4:1:7, 4:1:8, 4:1:9, 4:1:10, 5:1:1, 5:1:2, 5:1:3, 5:1:4, 5:1:5, 5:1:6, 5:1:7, 5:1:8, 5:1:9, 5:1:10, 6:1:1, 6:1:2, 6:1:3, 6:1:4, 6:1:5, 6:1:6, 6:1:7, 6:1:8, 6:1:9, or 6:1:10. In some embodiments, the ratio of cetuximab, binimetinib or a salt thereof, and palbociclib or a salt thereof by weight in the kits is about 20:3:6. In some embodiments, the ratio of cetuximab and binimetinib or a salt thereof by weight in the kits is the range of about 85:60 to 85:2. In some embodiments, the ratio of cetuximab and palbociclib or a salt thereof by weight in the kits is the range of about 17:25 to 34:3. In some embodiments, the ratio of binimetinib or a salt thereof and palbociclib or a salt thereof by weight in the kits is the range of about 20:125 to 60:75.

Also provided herein is a kit comprising osimertinib or salt thereof, binimetinib or a salt thereof, and palbociclib or a salt thereof. In some embodiments, the kit comprises a pharmaceutical composition comprising osimertinib or salt thereof and a pharmaceutically acceptable carrier, excipient, binder, or diluent. In some embodiments, the kit comprises a pharmaceutical composition comprising binimetinib or a salt thereof and a pharmaceutically acceptable carrier, excipient, binder, or diluent. In some embodiments, the kit comprises a pharmaceutical composition comprising palbociclib or a salt thereof and a pharmaceutically acceptable carrier, excipient, binder, or diluent.

In some embodiments, osimertinib or salt thereof, binimetinib or a salt thereof, and palbociclib or a salt thereof are formulated as one composition in the kit. In some embodiments, osimertinib or salt thereof, binimetinib or a salt thereof, and palbociclib or a salt thereof are formulated as two or more compositions in the kit. In some embodiments, osimertinib or salt thereof, binimetinib or a salt thereof, and palbociclib or a salt thereof are formulated separately. In some embodiments, binimetinib or a salt thereof, and/or palbociclib or a salt thereof are formulated for oral administration. In some embodiments, binimetinib or a salt thereof, and palbociclib or a salt thereof are formulated in the same form, such as solid or liquid form. In some embodiments, osimertinib or salt thereof, binimetinib or a salt thereof, and/or palbociclib or a salt thereof are formulated as solutions, emulsions, suspensions, dispersions, or inclusion complexes such as cyclodextrins in suitable pharmaceutical solvents or carriers, or as pills, tablets, lozenges, suppositories, sachets, dragees, granules, powders, powders for reconstitution, or capsules along with solid carriers according to conventional methods known in the art for preparation of various dosage forms. In some embodiments, osimertinib or salt thereof is formulated for administration via intravenous infusion. In some embodiments, binimetinib or a salt thereof and palbociclib or a salt thereof are formulated as one composition for oral administration.

In some embodiments, the ratios of osimertinib or salt thereof, binimetinib or a salt thereof, and palbociclib or a salt thereof by weight provided in the kits are about 1:1:1:, 2:1:1, 3:1:1, 4:1:1:, 1:2:1, 3:1:2, 3:1:3, 3:1:4, 3:1:5, 3:1:6, 3:1:7, 3:1:8, 3:1:9, 3:1:10, 4:1:2, 4:1:3, 4:1:4, 4:1:5, 4:1:6, 4:1:7, 4:1:8, 4:1:9, 4:1:10, 5:1:1, 5:1:2, 5:1:3, 5:1:4, 5:1:5, 5:1:6, 5:1:7, 5:1:8, 5:1:9, 5:1:10, 6:1:1, 6:1:2, 6:1:3, 6:1:4, 6:1:5, 6:1:6, 6:1:7, 6:1:8, 6:1:9, or 6:1:10. In some embodiments, the ratio of osimertinib or salt thereof, binimetinib or a salt thereof, and palbociclib or a salt thereof by weight in the kits is about 20:3:6. In some embodiments, the ratio of osimertinib or salt thereof and binimetinib or a salt thereof by weight in the kits is the range of about 85:60 to 85:2. In some embodiments, the ratio of osimertinib or salt thereof and palbociclib or a salt thereof by weight in the kits is the range of about 17:25 to 34:3. In some embodiments, the ratio of binimetinib or a salt thereof and palbociclib or a salt thereof by weight in the kits is the range of about 20:125 to 60:75.

Also provided herein is a kit comprising cetuximab, cobimetinib or a salt thereof, and abemaciclib or a salt thereof. In some embodiments, the kit comprises a pharmaceutical composition comprising cetuximab and a pharmaceutically acceptable carrier, excipient, binder, or diluent. In some embodiments, the kit comprises a pharmaceutical composition comprising cobimetinib or a salt thereof and a pharmaceutically acceptable carrier, excipient, binder, or diluent. In some embodiments, the kit comprises a pharmaceutical composition comprising abemaciclib or a salt thereof and a pharmaceutically acceptable carrier, excipient, binder, or diluent.

In some embodiments, cetuximab, cobimetinib or a salt thereof, and abemaciclib or a salt thereof are formulated as one composition in the kit. In some embodiments, cetuximab, cobimetinib or a salt thereof, and abemaciclib or a salt thereof are formulated as two or more compositions in the kit. In some embodiments, cetuximab, cobimetinib or a salt thereof, and abemaciclib or a salt thereof are formulated separately. In some embodiments, cobimetinib or a salt thereof, and/or abemaciclib or a salt thereof are formulated for oral administration. In some embodiments, cobimetinib or a salt thereof, and abemaciclib or a salt thereof are formulated in the same form, such as solid or liquid form. In some embodiments, cetuximab, cobimetinib or a salt thereof, and/or abemaciclib or a salt thereof are formulated as solutions, emulsions, suspensions, dispersions, or inclusion complexes such as cyclodextrins in suitable pharmaceutical solvents or carriers, or as pills, tablets, lozenges, suppositories, sachets, dragees, granules, powders, powders for reconstitution, or capsules along with solid carriers according to conventional methods known in the art for preparation of various dosage forms. In some embodiments, cetuximab is formulated for administration via intravenous infusion. In some embodiments, cobimetinib or a salt thereof and abemaciclib or a salt thereof are formulated as one composition for oral administration.

In some embodiments, the ratios of cetuximab, cobimetinib or a salt thereof, and abemaciclib or a salt thereof by weight provided in the kits are about 1:1:1:, 2:1:1, 3:1:1, 4:1:1:, 1:2:1, 3:1:2, 3:1:3, 3:1:4, 3:1:5, 3:1:6, 3:1:7, 3:1:8, 3:1:9, 3:1:10, 4:1:2, 4:1:3, 4:1:4, 4:1:5, 4:1:6, 4:1:7, 4:1:8, 4:1:9, 4:1:10, 5:1:1, 5:1:2, 5:1:3, 5:1:4, 5:1:5, 5:1:6, 5:1:7, 5:1:8, 5:1:9, 5:1:10, 6:1:1, 6:1:2, 6:1:3, 6:1:4, 6:1:5, 6:1:6, 6:1:7, 6:1:8, 6:1:9, or 6:1:10. In some embodiments, the ratio of cetuximab, cobimetinib or a salt thereof, and abemaciclib or a salt thereof by weight in the kits is about 20:3:6. In some embodiments, the ratio of cetuximab and cobimetinib or a salt thereof by weight in the kits is the range of about 85:60 to 85:2. In some embodiments, the ratio of cetuximab and abemaciclib or a salt thereof by weight in the kits is the range of about 17:25 to 34:3. In some embodiments, the ratio of cobimetinib or a salt thereof and abemaciclib or a salt thereof by weight in the kits is the range of about 20:125 to 60:75.

Also provided herein is a kit comprising osimertinib or a salt thereof, trametinib or a salt thereof, and palbociclib or a salt thereof. In some embodiments, the kit comprises a pharmaceutical composition comprising osimertinib or a salt thereof and a pharmaceutically acceptable carrier, excipient, binder, or diluent. In some embodiments, the kit comprises a pharmaceutical composition comprising trametinib or a salt thereof and a pharmaceutically acceptable carrier, excipient, binder, or diluent. In some embodiments, the kit comprises a pharmaceutical composition comprising palbociclib or a salt thereof and a pharmaceutically acceptable carrier, excipient, binder, or diluent.

In some embodiments, osimertinib or a salt thereof, trametinib or a salt thereof, and palbociclib or a salt thereof are formulated as one composition in the kit. In some embodiments, osimertinib or a salt thereof, trametinib or a salt thereof, and palbociclib or a salt thereof are formulated separately. In some embodiments, osimertinib or a salt thereof, trametinib or a salt thereof, and/or palbociclib or a salt thereof are formulated for oral administration. In some embodiments, osimertinib or a salt thereof, trametinib or a salt thereof, and palbociclib or a salt thereof are formulated in the same form, such as solid or liquid form. In some embodiments, osimertinib or a salt thereof, trametinib or a salt thereof, and/or palbociclib or a salt thereof are formulated as solutions, emulsions, suspensions, dispersions, or inclusion complexes such as cyclodextrins in suitable pharmaceutical solvents or carriers, or as pills, tablets, lozenges, suppositories, sachets, dragees, granules, powders, powders for reconstitution, or capsules along with solid carriers according to conventional methods known in the art for preparation of various dosage forms.

In some embodiments, the ratios of osimertinib or a salt thereof, trametinib or a salt thereof, and palbociclib or a salt thereof by weight provided in the kits are about 15:1:10, 20:1:10, 25:1:10, 30:1:10, 35:1:10, 40:1:10, 50:1:10, 15:1:20, 20:1:20, 25:1:20, 30:1:20, 35:1:20, 40:1:20, 50:1:20, 15:1:30, 20:1:30, 25:1:30, 30:1:30, 35:1:30, 40:1:30, 50:1:30 or 40:1:50. In some embodiments, the ratio of osimertinib or a salt thereof, trametinib or a salt thereof, and palbociclib or a salt thereof by weight in the compositions is about 15:1:12. In some embodiments, the ratio of osimertinib or a salt thereof and trametinib or a salt thereof by weight in the kits is the range of about 20:1 to 160:1. In some embodiments, the ratio of osimertinib or a salt thereof and palbociclib or a salt thereof by weight in the kits is the range of about 40:125 to 80:75. In some embodiments, the ratio of trametinib or a salt thereof and palbociclib or a salt thereof by weight in the kits is the range of about 1:250 to 2:75.

Also provided herein is a kit comprising cetuximab, TAK-733 or a salt thereof, and palbociclib or a salt thereof. In some embodiments, the kit comprises a pharmaceutical composition comprising cetuximab and a pharmaceutically acceptable carrier, excipient, binder, or diluent. In some embodiments, the kit comprises a pharmaceutical composition comprising TAK-733 or a salt thereof and a pharmaceutically acceptable carrier, excipient, binder, or diluent. In some embodiments, the kit comprises a pharmaceutical composition comprising palbociclib or a salt thereof and a pharmaceutically acceptable carrier, excipient, binder, or diluent.

Also provided herein is a kit comprising lapatinib or a salt thereof, trametinib or a salt thereof, and palbociclib or a salt thereof. In some embodiments, the kit comprises a pharmaceutical composition comprising lapatinib or a salt thereof and a pharmaceutically acceptable carrier, excipient, binder, or diluent. In some embodiments, the kit comprises a pharmaceutical composition comprising trametinib or a salt thereof and a pharmaceutically acceptable carrier, excipient, binder, or diluent. In some embodiments, the kit comprises a pharmaceutical composition comprising palbociclib or a salt thereof and a pharmaceutically acceptable carrier, excipient, binder, or diluent.

In some embodiments, lapatinib or a salt thereof, trametinib or a salt thereof, and palbociclib or a salt thereof are formulated as one composition in the kit. In some embodiments, lapatinib or a salt thereof, trametinib or a salt thereof, and palbociclib or a salt thereof are formulated separately. In some embodiments, lapatinib or a salt thereof, trametinib or a salt thereof, and/or palbociclib or a salt thereof are formulated for oral administration. In some embodiments, lapatinib or a salt thereof, trametinib or a salt thereof, and palbociclib or a salt thereof are formulated in the same form, such as solid or liquid form. In some embodiments, lapatinib or a salt thereof, trametinib or a salt thereof, and/or palbociclib or a salt thereof are formulated as solutions, emulsions, suspensions, dispersions, or inclusion complexes such as cyclodextrins in suitable pharmaceutical solvents or carriers, or as pills, tablets, lozenges, suppositories, sachets, dragees, granules, powders, powders for reconstitution, or capsules along with solid carriers according to conventional methods known in the art for preparation of various dosage forms.

In some embodiments, the ratios of lapatinib or a salt thereof, trametinib or a salt thereof, and palbociclib or a salt thereof by weight provided in the kits are about 1:1:1, 2:1:1, 3:1:1, 4:1:1, 1:2:1, 1:1:2, 1:1:3, 1:1:4, 1:1:5, 1:1:6, 1:1:7, 1:1:8, 1:1:9, 1:1:10, 2:1:2, 2:1:3, 2:1:4, 2:1:5, 2:1:6, 2:1:7, 2:1:8, 2:1:9, 2:1:10, 3:1:1, 3:1:2, 3:1:3, 3:1:4, 3:1:5, 3:1:6, 3:1:7, 3:1:8, 3:1:9, 3:1:10, 4:1:3, 5:1:3, 6:1:3, 7:1:3, 8:1:3, 9:1:3, 10:1:3, 20:1:3, or 30:1:3. In some embodiments, the ratio of lapatinib or a salt thereof, trametinib or a salt thereof, and palbociclib or a salt thereof by weight in the compositions is about 20:1:3. In some embodiments, the ratio of lapatinib or a salt thereof and trametinib or a salt thereof by weight is in the range of about 2:3 to 40:1. In some embodiments, the ratio of lapatinib or a salt thereof and palbociclib or a salt thereof by weight is in the range of about 2:3 to 20:1. In some embodiments, the ratio of trametinib or a salt thereof and palbociclib or a salt thereof by weight is in the range of about 20:125 to 60:75.

In another aspect, the kit further comprises a package insert including, without limitation, appropriate instructions for preparation and administration of the formulation, side effects of the formulation, and any other relevant information. The instructions may be in any suitable format, including, but not limited to, printed matter, videotape, computer readable disk, optical disc or directions to internet-based instructions.

In another aspect, kits for treating an individual who suffers from or is susceptible to the conditions described herein are provided, comprising a first container comprising a dosage amount of a composition or formulation as disclosed herein, and a package insert for use. The container may be any of those known in the art and appropriate for storage and delivery of intravenous formulation. In certain embodiments, the kit further comprises a second container comprising a pharmaceutically acceptable carrier, diluent, adjuvant, etc. for preparation of the formulation to be administered to the individual.

In another aspect, kits may also be provided that contain sufficient dosages of the compositions described herein (including pharmaceutical compositions thereof) to provide effective treatment for an individual for an extended period, such as 1-3 days, 1-5 days, a week, 2 weeks, 3, weeks, 4 weeks, 6 weeks, 8 weeks, 1 cycle, 2 cycles, 3 cycles, 4 cycles, 5 cycles, 6 cycles, 7 cycles, 8 cycles or more.

In some embodiments, the kits may also include multiple doses and may be packaged in quantities sufficient for storage and use in pharmacies, for example, hospital pharmacies and compounding pharmacies. In certain embodiments the kits may include a dosage amount of at least one composition as disclosed herein.

### EXAMPLES

The following examples are offered to illustrate but not to limit the invention. One of skill in the art will recognize that the following procedures may be modified using methods known to one of ordinary skill in the art.

### Example 1 In Vitro Test of Maximum Inhibition Index (MI) and Growth Inhibition Rate (GI %) for Cell Line with KRAS Mutations Treated with Solo and Combination Therapies

The experimental procedures are briefly outlined as follows. For in vitro drug testing, ˜3000 cells were exposed to a combination of drugs or single drugs for 4 days or 7 days.

Following exposure with drug, the cells were labeled with 5-ethynyl-2′-deoxyuridine (Edu) to assess the tumor cell proliferation rates. The labeling lasted 24 hours in the presence of drug exposure. In the control group, epithelial tumor cells received no drug exposure with media change (with 0.1% DMSO), but were similarly labeled with Edu. The labeled cells were fixed and blocked with 3% BSA and 0.5% Triton X-100 for 2 hours at room temperature, followed by staining with Hoechst 33342 and EpCAM antibody (1:4000) in PBS solution containing 0.5% Triton X-100 overnight at 4° C. Subsequently, the cells were rinsed with PBST then incubated with Alexa Fluor® 647 conjugated goat-anti-mouse secondary antibody for 30 minutes at room temperature.

The incorporated Edu was detected by Click-iT reaction where fixed cells were incubated with a reaction mixture containing 1× Click-iT Edu reaction buffer, CuSO₄, and azide-conjugated Alexa Fluor dye in the dark. The stained cells were washed with PBS two times before image acquisition and analysis.

For image acquisition and analysis, the stained tumor cells were imaged by a high-content screening (HCS) platform (Thermo Scientific CellomicsArrayScanXTi HCS reader). The 10× objective was used to collect images. Twenty-five fields were imaged for each well for the analysis. From the images three fluorescent signals were obtained from the HCS reader. Blue fluorescent signals recorded nucleus signals stained with Hoechst 33342, green fluorescent signal detected the Edu incorporated in newly synthesized DNA, and red fluorescent signal detected the EpCAM positive epithelial cells population.

The MI (Maximum Inhibition Index) was calculated using the EpCAM and Edu positive readout (Tables 1-2). MI=Edu positive cells in control/Edu positive cells in treatment. MI4 indicates an MI value after 4 days of treatment and MI7 indicates an MI values after 7 days of treatment.

The MI values and growth inhibition rate (GI %) for KRAS^{G12V}-CRC (SW620 and SW480) and KRAS^{G13D}-CRC (HCT-116) cell lines are shown in Tables 1 and 2, respectively. GI %=(1−1/MI)*100. Triplet combinations showed strong growth inhibition rate (in general G1%>90% after 4 days). The G1% for the triplet combinations is equal or higher than that for doublet combinations, and significantly higher than that for the singlet drug.

TABLE 1
Maximum Inhibition Index (MI) and Growth Inhibition Rate (GI %) during Solo
and Combination Therapies for KRAS^{G12V}-CRC (SW620 and SW480 cell lines)

| | Concentration | SW620 | | SW480 | |
|---|---|---|---|---|---|
| Samples | (uM) | GI % | MI 4 | GI % | MI4 |
| Osimertinib + Cobimetinib + Palbociclib | 0.5 + 0.4 + 0.15 | 97.30 | 37.03 | 94.32 | 17.60 |
| Osimertinib + Cobimetinib + Ribociclib | 0.5 + 0.4 + 5 | 99.49 | 195.39 | 99.86 | 739.18 |
| Osimertinib + Trametinib + Palbociclib | 0.5 + 0.02 + 0.15 | 95.66 | 23.04 | 86.24 | 7.27 |
| Osimertinib + Trametinib + Ribociclib | 0.5 + 0.02 + 5 | 97.73 | 44.03 | 99.40 | 166.15 |
| Afatinib + Trametinib + Palbociclib | 0.2 + 0.02 + 0.15 | 91.12 | 11.26 | 2.53 | 1.03 |
| Afatinib + Trametinib + Ribociclib | 0.2 + 0.02 + 5 | 95.60 | 22.74 | — | — |
| Afatinib + TAK-733 + Ribociclib | 0.2 + 0.18 + 5 | 97.83 | 46.14 | 99.43 | 176.22 |
| Afatinib + TAK-733 + Palbociclib | 0.2 + 0.18 + 0.15 | 97.18 | 35.46 | 89.90 | 9.90 |
| Osimertinib + TAK-733 + Ribociclib | 0.5 + 0.18 + 5 | 99.08 | 108.63 | 99.38 | 161.69 |
| Osimertinib + TAK-733 + Palbociclib | 0.5 + 0.18 + 0.15 | 97.30 | 37.09 | 86.38 | 7.34 |
| Osimertinib + TAK733 | 0.5 + 0.18 | 94.19 | 17.21 | 72.33 | 3.61 |
| Palbociclib + TAK-733 | 0.15 + 0.18 | 96.95 | 32.82 | 85.68 | 6.98 |
| Cobimetinib + Palbociclib | 0.4 + 0.15 | 96.83 | 31.58 | 90.50 | 10.53 |
| Osimertinib + Palbociclib | 0.5 + 0.15 | 75.76 | 4.13 | 48.85 | 1.96 |
| Osimertinib + Cobimetinib | 0.5 + 0.4 | 95.77 | 23.63 | 80.91 | 5.24 |
| TAK-733 | 0.18 | 88.39 | 8.61 | 67.79 | 3.10 |
| Trametinib | 0.02 | 70.41 | 3.38 | 62.07 | 2.64 |
| Ribociclib | 5 | 86.58 | 7.45 | 78.59 | 4.67 |
| Palbociclib | 0.15 | 67.69 | 3.10 | 40.51 | 1.68 |
| Cobimetinib | 0.4 | 91.84 | 12.25 | 79.85 | 4.96 |
| Afatinib | 0.2 | 26.78 | 1.37 | 21.08 | 1.27 |
| Osimertinib | 0.5 | 24.11 | 1.32 | 27.83 | 1.39 |
| Afatinib + Cobimetinib + Ribociclib | 0.2 + 0.4 + 5 | | | 99.68 | 314.83 |
| Ribociclib + TAK733 | 5 + 0.18 | | | 98.74 | 79.65 |
| Cobimetinib + Ribociclib | 0.4 + 5 | | | 99.31 | 145.81 |
| Osimertinib + Ribociclib | 0.5 + 5 | | | 84.24 | 6.34 |
| Osimertinib + Trametinib | 0.5 + 0.02 | | | 70.56 | 3.40 |

TABLE 2
Maximum Inhibition Index (MI) and Growth Inhibition
Rate (GI %) during Solo and Combination Therapies for
KRAS^{G13D}-CRC (HCT-116 cell line)

| | Concentration | HCT-116 | |
|---|---|---|---|
| Samples | uM | GI % | MI 4 |
| Osimertinib + Cobimetinib + | 0.5 + 0.4 + 0.15 | 99.81 | 519.94 |
| Palbociclib | | | |
| Osimertinib + Cobimetinib + | 0.5 + 0.4 + 5 | 99.88 | 826.10 |
| Ribociclib | | | |
| Osimertinib + Trametinib + | 0.5 + 0.02 + 0.15 | 98.80 | 83.07 |
| Palbociclib | | | |
| Osimertinib + Trametinib + | 0.5 + 0.02 + 5 | 99.48 | 192.95 |
| Ribociclib | | | |
| Afatinib + Trametinib + | 0.2 + 0.02 + 0.15 | 95.91 | 24.47 |
| Palbociclib | | | |
| Afatinib + Trametinib + | 0.2 + 0.02 + 5 | 99.26 | 135.19 |
| Ribociclib | | | |
| Afatinib + TAK-733 + Ribociclib | 0.2 + 0.18 + 5 | 99.54 | 217.08 |
| Afatinib + TAK-733 + Palbociclib | 0.2 + 0.18 + 0.15 | 98.98 | 98.51 |
| Osimertinib + TAK-733 + | 0.5 + 0.18 + 5 | 99.58 | 239.46 |
| Ribociclib | | | |
| Osimertinib + TAK-733 + | 0.5 + 0.18 + 0.15 | 98.83 | 85.64 |
| Palbociclib | | | |
| Osimertinib + TAK733 | 0.5 + 0.18 | 96.28 | 26.90 |
| Palbociclib + TAK-733 | 0.15 + 0.18 | 98.86 | 87.46 |
| Cobimetinib + Palbociclib | 0.4 + 0.15 | 99.57 | 234.42 |
| Osimertinib + Palbociclib | 0.5 + 0.15 | 82.86 | 5.83 |
| Osimertinib + Cobimetinib | 0.5 + 0.4 | 99.57 | 231.20 |
| TAK-733 | 0.18 | 90.18 | 10.18 |
| Trametinib | 0.02 | 76.37 | 4.23 |
| Ribociclib | 5 | 85.49 | 6.89 |
| Palbociclib | 0.15 | 71.62 | 3.52 |
| Cobimetinib | 0.4 | 98.13 | 53.44 |
| Afatinib | 0.2 | 13.99 | 1.16 |
| Osimertinib | 0.5 | 27.62 | 1.38 |

### Example 2 Ex Vivo Drug Tests Using Conditional Reprogramming Cell Pools (PDX Model)

Surgical specimen from colorectal cancer patients were obtained from a hospital after receiving patient consensus. Patient-derived xenograft tumor specimens were obtained from Nod/SCID mice inoculated with surgical tumor specimen from patients.

For ex vivo drug sensitivity assays, the colorectal tumor cells (CRC) were isolated from the patient tissue sample or PDX xenograft tumor tissue. Briefly, the tumor tissues were cut into small pieces less than 1 mm in diameter using scissors. The tumor fragments were transferred into a sterile 100-ml triangle glass flask loaded with a magnet stir bar. A 10-15 ml digestion media containing 0.25 U/ml Liberase DH was added into the minced tumor tissues to start enzyme digestion. The enzyme mixture was incubated at 37° C. for 1-2 hours with moderate stirring. The digested tumor tissue was filtered through a 100-μm cell retainer. The filtrates were re-filtered through a 40-μm cell restrainer. The CRC clusters retained on the 40-μm cell restrainer was collected, wash twice with HBSS, then re-suspended in a defined growth media supplemented with several stem cell growth factors.

The CRC clusters retained on the 40-μm cell restrainer were collected, wash twice with HBSS, then re-suspended in a defined growth media supplemented with cell growth factors and small molecule inhibitors. The CRC clusters were recovered in a defined growth medium overnight. The defined growth medium was StemPro® hESC SFM (defined, serum- and feeder-free medium (SFM)) supplemented with: Nicotinamide, Wnt3A, Noggin (Bone Morphogenetic Protein (BMP) inhibitor), Rspondin-1 (Wnt/β-catenin signaling agonist), and Y27632 (Rho-associated, coiled-coil containing protein kinase (ROCK-1) inhibitor).

For ex vivo drug testing, 1,000-5,000 CRCs were seeded and subsequently exposed to various combinations of drugs for 4 or 7 days. The combinations were prepared in DMSO with a final DMSO concentration of 0.1% in media.

Following exposure with drug, the CRCs were labeled with 5-ethynyl-2′-deoxyuridine (Edu) to assess the tumor cell proliferation rates. The labeling lasted 24 hours in the presence of drug exposure. In the control group, epithelial tumor cells received no drug exposure with each change of media (containing 0.1% DMSO), but were otherwise similarly labeled with Edu. The labeled cells were fixed and blocked with 3% BSA and 0.5% Triton X-100 for 2 hours at room temperature, followed by staining with Hoechst 33342 and EpCAM antibody (1:4000) in PBS solution containing 0.5% Triton X-100 overnight at 4° C. Subsequently, the cells were rinsed with PBST then incubated with Alexa Fluor® 647 conjugated goat-anti-mouse secondary antibody for 30 minutes at room temperature.

The incorporated Edu was detected by Click-iT reaction where fixed cells were incubated with a reaction mixture containing 1× Click-iT Edu reaction buffer, CuSO₄, and azide-conjugated Alexa Fluor dye in the dark. The stained cells were washed with PBS two times before image acquisition and analysis.

The MI (Maximum Inhibition Index) was calculated using the EpCAM and Edu positive readout (Table 8). MI=Edu positive cells in control/Edu positive cells in treatment. Growth inhibition rate (GI %)=(1−1/MI)*100. Triplet combinations inhibited growth of patient-derived cells lines harboring various KRAS mutations at higher or equal GI % when compared to doublet combinations, and at significantly higher GI % compared to singlet treatments (mono- or solo-drug).

The MI and GI % values for KRAS^{G12V}-PDX (NYP-031, NYP-028 and NYL-205), KRAS^{G12D}-PDX (NYL-233, NYP-044 and CKY-041), KRAS^{G13D}-PDX (NYL-178, NYL-GZ-085, NYL-GZ-094), KRAS^{G12C}-PDX (NYL-229 and NYL-194), KRAS^{G12A}-PDX (NYL-HEB-061) and KRAS^{WT}-PDX (NYL-161) are shown in Tables 3-9, respectively.

TABLE 3
Maximum Inhibition Index (MI) and Growth Inhibition Rate (GI %)
during Solo and Combination Therapies for KRAS^{G12V}-CRC (PDX)

| | Concentration | NYP-031 | | NYP-028 | | NYL-205 | |
|---|---|---|---|---|---|---|---|
| Samples | (uM) | GI % | MI 7 | GI % | MI 7 | GI % | MI 7 |
| Osimertinib + Cobimetinib + | 0.5 + 0.4 + 0.15 | 50.86 | 2.03 | 98.68 | 75.64 | 100.00 | 13148.55 |
| Palbociclib | | | | | | | |
| Osimertinib + Cobimetinib + | 0.5 + 0.4 + 5 | 73.27 | 3.74 | 96.21 | 26.38 | 100.00 | 13148.55 |
| Ribociclib | | | | | | | |
| Osimertinib + Trametinib + | 0.5 + 0.02 + 0.15 | 23.72 | 1.31 | 97.54 | 40.59 | — | — |
| Palbociclib | | | | | | | |
| Osimertinib + Trametinib + | 0.5 + 0.02 + 5 | 26.33 | 1.36 | 98.36 | 60.99 | — | — |
| Ribociclib | | | | | | | |
| Afatinib + Trametinib + Palbociclib | 0.2 + 0.02 + 0.15 | 15.32 | 1.18 | 8.33 | 1.09 | — | — |
| Afatinib + Trametinib + Ribociclib | 0.2 + 0.02 + 5 | — | — | — | — | — | — |
| Afatinib + TAK-733 + Ribociclib | 0.2 + 0.18 + 5 | 74.89 | 3.98 | 95.74 | 23.48 | 99.97 | 3291.25 |
| Afatinib + TAK-733 + Palbociclib | 0.2 + 0.18 + 0.15 | 59.54 | 2.47 | 97.56 | 40.91 | 99.89 | 932.85 |
| Osimertinib + TAK-733 + Ribociclib | 0.5 + 0.18 + 5 | 58.63 | 2.42 | 97.16 | 35.26 | 100.00 | 13148.55 |
| Osimertinib + TAK-733 + Palbociclib | 0.5 + 0.18 + 0.15 | 36.63 | 1.58 | 97.01 | 33.46 | 99.90 | 1028.03 |
| Osimertinib + TAK733 | 0.5 + 0.18 | 7.01 | 1.08 | 94.73 | 18.99 | 99.01 | 101.28 |
| Palbociclib + TAK-733 | 0.15 + 0.18 | 3.57 | 1.04 | 45.44 | 1.83 | 32.73 | 1.49 |
| Cobimetinib + Palbociclib | 0.4 + 0.15 | 2.68 | 1.03 | 22.49 | 1.29 | 55.46 | 2.24 |
| Osimertinib + Palbociclib | 0.5 + 0.15 | −85.58 | 0.54 | 11.89 | 1.13 | 92.58 | 13.48 |
| Osimertinib + Cobimetinib | 0.5 + 0.4 | 34.01 | 1.52 | 97.38 | 38.16 | 99.69 | 327.75 |
| TAK-733 | 0.18 | −15.90 | 0.86 | 9.31 | 1.10 | −0.06 | 1.00 |
| Trametinib | 0.02 | 16.38 | 1.20 | −33.32 | 0.75 | −4.47 | 0.96 |
| Ribociclib | 5 | 1.44 | 1.01 | 67.26 | 3.05 | 63.99 | 2.78 |
| Palbociclib | 0.15 | 2.94 | 1.03 | 41.26 | 1.70 | 31.10 | 1.45 |
| Cobimetinib | 0.4 | −25.67 | 0.80 | −7.70 | 0.93 | 27.39 | 1.38 |
| Afatinib | 0.2 | −48.75 | 0.67 | −75.81 | 0.57 | 75.40 | 4.06 |
| Osimertinib | 0.5 | −117.79 | 0.46 | −86.23 | 0.54 | 65.96 | 2.94 |
| Afatinib + Cobimetinib + Ribociclib | 0.2 + 0.4 + 5 | 73.22 | 3.73 | 94.09 | 16.91 | 100.00 | 13148.55 |
| Ribociclib + TAK733 | 5 + 0.18 | 1.67 | 1.02 | 59.64 | 2.48 | 70.70 | 3.41 |
| Cobimetinib + Ribociclib | 0.4 + 5 | 18.60 | 1.23 | 65.20 | 2.87 | 73.37 | 3.76 |
| Osimertinib + Ribociclib | 0.5 + 5 | −25.39 | 0.80 | 68.99 | 3.22 | 96.91 | 32.41 |
| Osimertinib + Trametinib | 0.5 + 0.02 | 0.42 | 1.00 | 92.51 | 13.36 | — | — |
| Binimetinib | 0.2 | | | | | −3.06 | 0.97 |
| Osimertinib + Binimetinib | 0.5 + 0.2 | | | | | 98.30 | 58.74 |
| Afatinib + Cobimetinib + Palbociclib | 0.2 + 0.4 + 0.15 | | | | | 100.00 | 13148.55 |
| Binimetinib + Ribociclib | 0.2 + 5 | | | | | 65.72 | 2.92 |

TABLE 4
Maximum Inhibition Index (MI) and Growth Inhibition Rate (GI %)
during Solo and Combination Therapies for KRAS^{G12D}-CRC (PDX)

| | Concentration | NYL-233 | | NYP-044 | | CKY-041 | |
|---|---|---|---|---|---|---|---|
| Samples | (uM) | GI % | MI 7 | GI % | MI 7 | GI % | MI 7 |
| Osimertinib + Cobimetinib + | 0.5 + 0.4 + 0.15 | 79.86 | 4.96 | 48.28 | 1.93 | 99.85 | 666.63 |
| Palbociclib | | | | | | | |
| Osimertinib + Cobimetinib + | 0.5 + 0.4 + 5 | 90.81 | 10.88 | 76.14 | 4.19 | 99.75 | 407.57 |
| Ribociclib | | | | | | | |
| Osimertinib + Trametinib + | 0.5 + 0.02 + 0.15 | 72.14 | 3.59 | 37.13 | 1.59 | 98.84 | 86.01 |
| Palbociclib | | | | | | | |
| Osimertinib + Trametinib + | 0.5 + 0.02 + 5 | 79.02 | 4.77 | 64.00 | 2.78 | 99.08 | 108.33 |
| Ribociclib | | | | | | | |
| Osimertinib + Encorafinib + | 0.5 + 0.6 + 0.2 | | | | | 65.20 | 2.87 |
| Binimetnib | | | | | | | |
| Afatinib + Trametinib + | 0.2 + 0.02 + 0.15 | 73.98 | 3.84 | 8.37 | 1.09 | — | — |
| Palbociclib | | | | | | | |
| Afatinib + Trametinib + | 0.2 + 0.02 + 5 | 86.59 | 7.46 | — | — | — | — |
| Ribociclib | | | | | | | |
| Afatinib + TAK-733 + | 0.2 + 0.18 + 5 | 88.31 | 8.55 | 57.73 | 2.37 | 99.24 | 131.93 |
| Ribociclib | | | | | | | |
| Afatinib + TAK-733 + | 0.2 + 0.18 + 0.15 | 79.33 | 4.84 | 34.08 | 1.52 | 98.96 | 96.57 |
| Palbociclib | | | | | | | |
| Osimertinib + TAK-733 + | 0.5 + 0.18 + 5 | 84.18 | 6.32 | 65.53 | 2.90 | 99.19 | 123.21 |
| Ribociclib | | | | | | | |
| Osimertinib + TAK-733 + | 0.5 + 0.18 + 0.15 | 77.69 | 4.48 | 32.65 | 1.48 | 98.87 | 88.82 |
| Palbociclib | | | | | | | |
| Osimertinib + TAK733 | 0.5 + 0.18 | 50.59 | 2.02 | 28.20 | 1.39 | 97.38 | 38.12 |
| Palbociclib + TAK-733 | 0.15 + 0.18 | 7.70 | 1.08 | 37.81 | 1.61 | 7.61 | 1.08 |
| Cobimetinib + Palbociclib | 0.4 + 0.15 | 19.22 | 1.24 | 39.18 | 1.64 | −3.64 | 0.96 |
| Osimertinib + Palbociclib | 0.5 + 0.15 | 13.45 | 1.16 | 42.78 | 1.75 | 80.72 | 5.19 |
| Osimertinib + Cobimetinib | 0.5 + 0.4 | 67.47 | 3.07 | 48.25 | 1.93 | 99.31 | 145.40 |
| TAK-733 | 0.18 | −3.08 | 0.97 | 25.59 | 1.34 | 11.57 | 1.13 |
| Trametinib | 0.02 | −7.53 | 0.93 | 9.18 | 1.10 | −42.05 | 0.70 |
| Ribociclib | 5 | 14.94 | 1.18 | 60.73 | 2.55 | 9.50 | 1.10 |
| Palbociclib | 0.15 | 0.94 | 1.01 | 36.85 | 1.58 | −5.35 | 0.95 |
| Cobimetinib | 0.4 | −4.60 | 0.96 | 13.58 | 1.16 | −20.92 | 0.83 |
| Afatinib | 0.2 | −10.30 | 0.91 | 12.82 | 1.15 | 30.29 | 1.43 |
| Osimertinib | 0.5 | −4.68 | 0.96 | 10.16 | 1.11 | 32.97 | 1.49 |
| Afatinib + Cobimetinib + | 0.2 + 0.4 + 5 | | | 63.55 | 2.74 | 99.77 | 429.43 |
| Ribociclib | | | | | | | |
| Ribociclib + TAK733 | 5 + 0.18 | | | 54.11 | 2.18 | 1.65 | 1.02 |
| Cobimetinib + Ribociclib | 0.4 + 5 | | | 52.27 | 2.10 | 22.18 | 1.29 |
| Osimertinib + Ribociclib | 0.5 + 5 | | | 63.70 | 2.75 | 75.68 | 4.11 |
| Osimertinib + Trametinib | 0.5 + 0.02 | | | 28.18 | 1.39 | 95.49 | 22.17 |

As shown in Table 4, the triplet combination of osimertinib, cobimetinib and palbociclib, which are an EGFR inhibitor, an MEK1/2 inhibitor and a CDK4/6 inhibitor, respectively, showed significantly higher maximum inhibition (MI) and growth inhibition rate (GI %) for a patient-derived KRASG12D-CRC cell line of CKY-041 than the triplet combination of osimertinib, encorafenib, and binimetinib, which are an EGFR inhibitor, a BRAF inhibitor, and an NMEK1/2 inhibitor, respectively.

TABLE 5
Maximum Inhibition Index (MI) and Growth
Inhibition Rate (GI %) during Solo and Combination
Therapies for KRAS^{G13D}-CRC (PDX)

| | Concentration | H358 | | H23 | |
|---|---|---|---|---|---|
| Samples | (uM) | GI % | MI 4 | GI % | MI 4 |
| Osimertinib + | 0.5 + 0.4 + 0.15 | 99.88 | 810.84 | 89.43 | 9.46 |
| Cobimetinib + | | | | | |
| Palbociclib | | | | | |
| Osimertinib + | 0.5 + 0.6 + 0.2 | 88.96 | 9.06 | 69.56 | 3.29 |
| Encorafinib + | | | | | |
| Binimetinib | | | | | |

As shown in Table 5, the triplet combination of osimertinib, cobimetinib and palbociclib, which are an EGFR inhibitor, an MEK1/2 inhibitor and a CDK4/6 inhibitor, respectively, showed significantly maximum inhibition (MI) and growth inhibition rate (GI %) for a patient-derived KRAS^{G13D}-CRC cell line of NYL-GZ-085.

TABLE 6
Maximum Inhibition Index (MI) and Growth Inhibition Rate (GI %)
during Solo and Combination Therapies for KRAS^{G12C}-CRC (PDX)

| | Concentration | NYL-229 | | NYP-194 | |
|---|---|---|---|---|---|
| Samples | (uM) | TGI % | MI 7 | TGI % | MI 7 |
| Osimertinib + Cobimetinib + Palbociclib | 0.5 + 0.4 + 0.15 | 84.35 | 6.39 | 96.11 | 25.68 |
| Osimertinib + Cobimetinib + Ribociclib | 0.5 + 0.4 + 5 | 94.53 | 18.29 | 94.88 | 19.53 |
| Osimertinib + Trametinib + Palbociclib | 0.5 + 0.02 + 0.15 | 1.40 | 1.01 | 86.54 | 7.43 |
| Osimertinib + Trametinib + Ribociclib | 0.5 + 0.02 + 5 | 41.60 | 1.71 | 85.98 | 7.13 |
| Afatinib + Trametinib + Palbociclib | 0.2 + 0.02 + 0.15 | −38.05 | 0.72 | 5.27 | 1.06 |
| Afatinib + Trametinib + Ribociclib | 0.2 + 0.02 + 5 | 35.54 | 1.55 | — | — |
| Afatinib + TAK-733 + Ribociclib | 0.2 + 0.18 + 5 | 47.00 | 1.89 | 95.04 | 20.15 |
| Afatinib + TAK-733 + Palbociclib | 0.2 + 0.18 + 0.15 | 10.77 | 1.12 | 86.14 | 7.21 |
| Osimertinib + TAK-733 + Ribociclib | 0.5 + 0.18 + 5 | 67.09 | 3.04 | 93.69 | 15.84 |
| Osimertinib + TAK-733 + Palbociclib | 0.5 + 0.18 + 0.15 | −0.94 | 0.99 | 83.68 | 6.13 |
| Osimertinib + TAK733 | 0.5 + 0.18 | −2.60 | 0.97 | 73.51 | 3.77 |
| Palbociclib + TAK-733 | 0.15 + 0.18 | −39.04 | 0.72 | 60.13 | 2.51 |
| Cobimetinib + Palbociclib | 0.4 + 0.15 | −35.21 | 0.74 | 46.50 | 1.87 |
| Osimertinib + Palbociclib | 0.5 + 0.15 | −50.51 | 0.66 | 26.58 | 1.36 |
| Osimertinib + Cobimetinib | 0.5 + 0.4 | 73.50 | 3.77 | 90.45 | 10.47 |
| TAK-733 | 0.18 | −15.53 | 0.87 | 41.27 | 1.70 |
| Trametinib | 0.02 | −38.39 | 0.72 | 3.89 | 1.04 |
| Ribociclib | 5 | 31.91 | 1.47 | 57.53 | 2.35 |
| Palbociclib | 0.15 | 3.78 | 1.04 | 24.72 | 1.33 |
| Cobimetinib | 0.4 | −23.11 | 0.81 | 36.07 | 1.56 |
| Afatinib | 0.2 | −59.14 | 0.63 | 11.92 | 1.14 |
| Osimertinib | 0.5 | −70.94 | 0.58 | 12.01 | 1.14 |
| Afatinib + Cobimetinib + Ribociclib | 0.2 + 0.4 + 5 | | | 93.79 | 16.10 |
| Ribociclib + TAK733 | 5 + 0.18 | | | 61.09 | 2.57 |
| Cobimetinib + Ribociclib | 0.4 + 5 | | | 59.53 | 2.47 |
| Osimertinib + Ribociclib | 0.5 + 5 | | | 58.23 | 2.39 |
| Osimertinib + Trametinib | 0.5 + 0.02 | | | 55.79 | 2.26 |

TABLE 7
Maximum Inhibition Index (MI) and Growth
Inhibition Rate (GI %) during Solo and Combination
Therapies for KRAS^{G12A}-CRC (PDX)

| | Concentration | NYL-HEB-061 | |
|---|---|---|---|
| Samples | (uM) | GI % | MI 7 |
| Osimertinib + Cobimetinib + | 0.5 + 0.4 + 0.15 | 57.89 | 2.37 |
| Palbociclib | | | |
| Osimertinib + Cobimetinib + | 0.5 + 0.4 + 5 | 79.00 | 4.76 |
| Ribociclib | | | |
| Osimertinib + Trametinib + | 0.5 + 0.02 + 0.15 | 35.42 | 1.55 |
| Palbociclib | | | |
| Osimertinib + Trametinib + | 0.5 + 0.02 + 5 | 45.74 | 1.84 |
| Ribociclib | | | |
| Afatinib + Trametinib + | 0.2 + 0.02 + 0.15 | 28.29 | 1.39 |
| Palbociclib | | | |
| Afatinib + Trametinib + | 0.2 + 0.02 + 5 | 35.82 | 1.56 |
| Ribociclib | | | |
| Afatinib + TAK-733 + Ribociclib | 0.2 + 0.18 + 5 | 63.53 | 2.74 |
| Afatinib + TAK-733 + Palbociclib | 0.2 + 0.18 + 0.15 | 52.70 | 2.11 |
| Osimertinib + TAK-733 + | 0.5 + 0.18 + 5 | 53.96 | 2.17 |
| Ribociclib | | | |
| Osimertinib + TAK-733 + | 0.5 + 0.18 + 0.15 | 36.57 | 1.58 |
| Palbociclib | | | |
| Osimertinib + TAK733 | 0.5 + 0.18 | 20.35 | 1.26 |
| Palbociclib + TAK-733 | 0.15 + 0.18 | 10.72 | 1.12 |
| Cobimetinib + Palbociclib | 0.4 + 0.15 | 11.97 | 1.14 |
| Osimertinib + Palbociclib | 0.5 + 0.15 | 21.84 | 1.28 |
| Osimertinib + Cobimetinib | 0.5 + 0.4 | 62.99 | 2.70 |
| TAK-733 | 0.18 | −0.69 | 0.99 |
| Trametinib | 0.02 | 4.91 | 1.05 |
| Ribociclib | 5 | 60.88 | 2.56 |
| Palbociclib | 0.15 | 20.98 | 1.27 |
| Cobimetinib | 0.4 | 22.18 | 1.29 |
| Afatinib | 0.2 | 16.06 | 1.19 |
| Osimertinib | 0.5 | 26.18 | 1.35 |

TABLE 8
Maximum Inhibition Index (MI) and Growth Inhibition Rate (GI
%) during Solo and Combination Therapies for KRAS^{WT}-CRC (PDX)

| | Concentration | NYL-161 | |
|---|---|---|---|
| Samples | uM | GI % | MI 7 |
| Osimertinib + Cobimetinib + | 0.5 + 0.4 + 0.15 | 98.54 | 68.41 |
| Palbociclib | | | |
| Osimertinib + Cobimetinib + | 0.5 + 0.4 + 5 | 99.66 | 292.34 |
| Ribociclib | | | |
| Osimertinib + Trametinib + | 0.5 + 0.02 + 0.15 | — | — |
| Palbociclib | | | |
| Osimertinib + Trametinib + | 0.5 + 0.02 + 5 | — | — |
| Ribociclib | | | |
| Afatinib + Trametinib + Palbociclib | 0.2 + 0.02 + 0.15 | — | — |
| Afatinib + Trametinib + Ribociclib | 0.2 + 0.02 + 5 | — | — |
| Afatinib + TAK-733 + Ribociclib | 0.2 + 0.18 + 5 | 99.92 | 1195.45 |
| Afatinib + TAK-733 + Palbociclib | 0.2 + 0.18 + 0.15 | 98.84 | 86.07 |
| Osimertinib + TAK-733 + Ribociclib | 0.5 + 0.18 + 5 | 99.43 | 174.83 |
| Osimertinib + TAK-733 + | 0.5 + 0.18 + 0.15 | 97.29 | 36.94 |
| Palbociclib | | | |
| Vemurafenib + TAK733 | 5 + 0.18 | 24.91 | 1.33 |
| Osimertinib + TAK733 | 0.5 + 0.18 | 87.38 | 7.92 |
| Palbociclib + TAK-733 | 0.15 + 0.18 | 52.19 | 2.09 |
| Cobimetinib + Palbociclib | 0.4 + 0.15 | 51.45 | 2.06 |
| Osimertinib + Palbociclib | 0.5 + 0.15 | 92.77 | 13.83 |
| Osimertinib + Cobimetinib | 0.5 + 0.4 | 89.80 | 9.80 |
| TAK-733 | 0.18 | 15.32 | 1.18 |
| Trametinib | 0.02 | 9.54 | 1.11 |
| Ribociclib | 5 | 64.27 | 2.80 |
| Palbociclib | 0.15 | 45.15 | 1.82 |
| Cobimetinib | 0.4 | 18.48 | 1.23 |
| Afatinib | 0.2 | 89.21 | 9.27 |
| Osimertinib | 0.5 | 79.59 | 4.90 |
| Afatinib + Cobimetinib + Ribociclib | 0.2 + 0.4 + 5 | 99.95 | 1916.59 |
| Ribociclib + TAK733 | 5 + 0.18 | 60.97 | 2.56 |
| Cobimetinib + Ribociclib | 0.4 + 5 | 67.20 | 3.05 |
| Osimertinib + Ribociclib | 0.5 + 5 | 96.29 | 26.97 |
| Osimertinib + Trametinib | 0.5 + 0.02 | — | — |
| Binimetinib | 0.2 | 11.55 | 1.13 |
| Osimertinib + Binimetinib | 0.5 + 0.2 | 84.70 | 6.53 |
| Afatinib + Cobimetinib + Palbociclib | 0.2 + 0.4 + 0.15 | 98.43 | 63.75 |
| Binimetinib + Ribociclib | 0.2 + 5 | 61.78 | 2.62 |

### Example 3 Tumor Volume Reduction in Mice During Combination Therapy in a PDX Model with Patient CKY041-P1

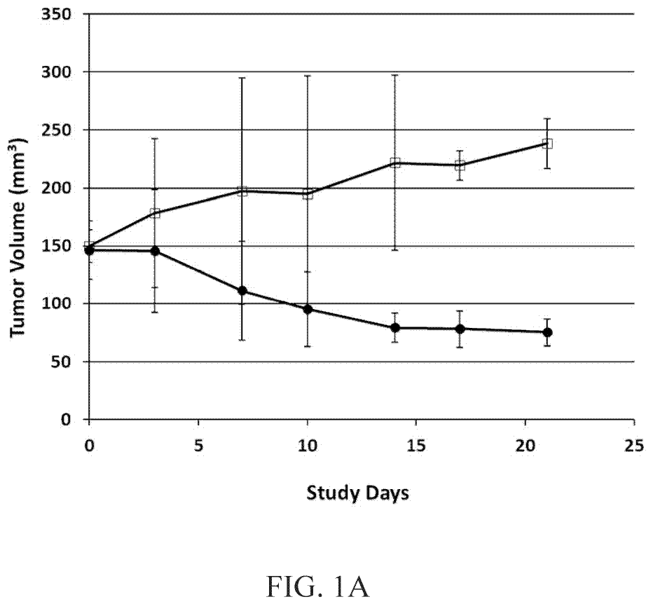

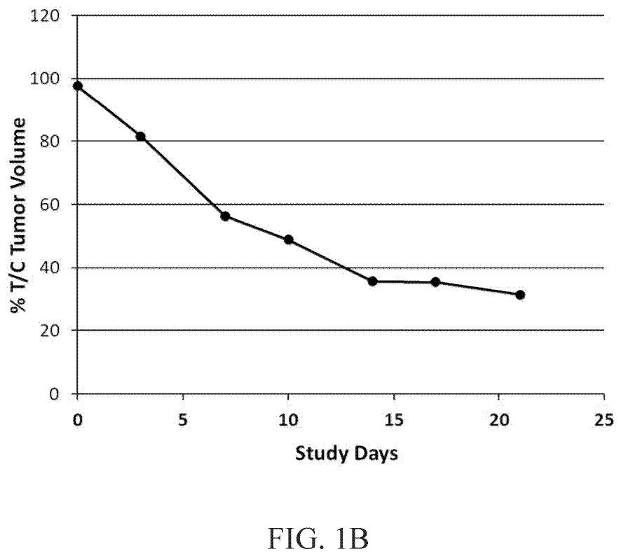

The study was conducted in xenograft tumor PDX model established with surgical tumor tissues from KRAS^{G12D}-CRC patient CKY041-P1. The combination therapy scheme is shown in Table 9. Mean tumor volumes during the indicated combination therapy are shown in FIG. 1A and Table 10. Briefly, 6-8 week-old female NOD/SCID mice were used for the studies. Tumor samples obtained from patients were immediately transferred into tissue preservation solution and sliced into small fragments. Mice were inoculated with the fragments subcutaneously at one flank to produce xenografts called passage 1 (P1). The serial xenografts of different passages were generated using the same procedure. When the average tumor size reached approximately 250-300 mm³ in the mice, the animals were randomly allocated into different groups, with 5 mice per group. The day of randomization was defined as study day 0. Tumor volume is expressed in mm³ using the following formula: V (volume)=(a×b²)/2 where a and b are the long and short diameters of the tumor, respectively.

The mean tumor volumes during the combination therapy are shown in FIG. 1A and Table 10. The mean tumor volume in treated mice (mean (T)) compared to that of control mice (mean(C)) (% T/C Tumor Volume=mean(T)/mean(C)*100%) is shown in FIG. 1B and Table 11. The percentage inhibition of tumor volume in test mice compared to that of control mice (Mean % Inhibition=(mean(C)−mean(T))/mean(C)*100%) was also measured. The tumor inhibition over time compared to initial tumor volume (mean(TO)) in treated mice was compared to that of control mice (% ΔT/ΔC=(mean(Ti)−mean(T0))/(mean(Ci)−mean(C0))*100. The percentage tumor inhibition over time compared to initial tumor volume (mean(T0)) in treated mice as compared to control mice (Mean % ΔInhibition=((mean(Ci)−mean(C0))−(mean(Ti)−mean(T0)))/(mean(Ci)−mean(C0))*100%) was also calculated. The individual tumor volumes in surviving mice in each group, if any, at the end of the study were also recorded. Tumor growth inhibition %=[1−(tumor size of treatment at the end of the study−tumor size of treatment at the start of study)/(tumor size of vehicle control at the end of study-tumor size at the start of study)]*100%. Tumor regression parameter is calculated as % REG=[(tumor volume of the treatment at the start of the study−tumor volume of the treatment at the end of the study)/tumor volume of the treatment at the start of the study]*100%, At Day 28 (after 1^{st} cycle of treatment), the triplet treatment with Lapatinib, 100 mg/kg, QD*21 days; Trametinib, 0.3 mg/kg, QD*21 days; Palbociclib, 75 mg/kg, QD*21 days in Table 10 showed tumor growth inhibition at % REG of 31%.

TABLE 9
Mice groups for Control and Combination
Therapy in CKY041-P1 PDX

| Group | Combination Therapy Scheme |
|---|---|
| Group 01 | Control, 0 mg/kg, QW*3 weeks |
| Group 02 | Lapatinib, 100 mg/kg, QD*21 days; |
| | Trametinib, 0.3 mg/kg, QD*21 days; |
| | Palbociclib, 75 mg/kg, QD*21 days |

TABLE 10
Mean tumor volume (mm³) in CKY041-P1 PDX

| Day | 0 | 3 | 7 | 10 | 14 | 17 | 21 | 24 | 28 |
|---|---|---|---|---|---|---|---|---|---|
| Control | 149.72 ± | 178.35 ± | 197.25 ± | 194.88 ± | 221.64 ± | 219.45 ± | 238.20 ± | 242.92 ± | 284.75 ± |
| | 14.05 | 64.50 | 97.87 | 101.51 | 75.51 | 12.60 | 21.36 | 35.72 | 71.90 |
| Group | 146.11 ± | 145.45 ± | 111.12 ± | 95.10 ± | 79.06 ± | 77.94 ± | 75.13 ± | 95.58 ± | 101.83 ± |
| 02 | 25.26 | 53.26 | 42.99 | 32.21 | 12.41 | 15.77 | 11.37 | 31.65 | 18.44 |

TABLE 11
Mean tumor volume in treated mice versus
control mice (% T/C) in CKY041-P1 PDX

| Day | 0 | 3 | 7 | 10 | 14 | 17 | 21 | 24 | 28 |
|---|---|---|---|---|---|---|---|---|---|
| Group | 97.59 | 81.55 | 56.33 | 48.80 | 35.67 | 35.52 | 31.54 | 39.35 | 35.76 |
| 02 | | | | | | | | | |

### Example 4 Tumor Volume Reduction in Mice During Combination Therapy in a PDX Model with Patient NYL170-P2

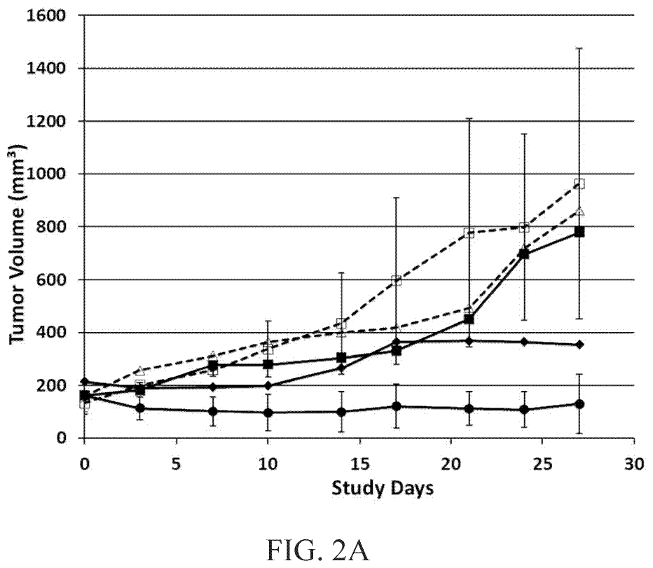

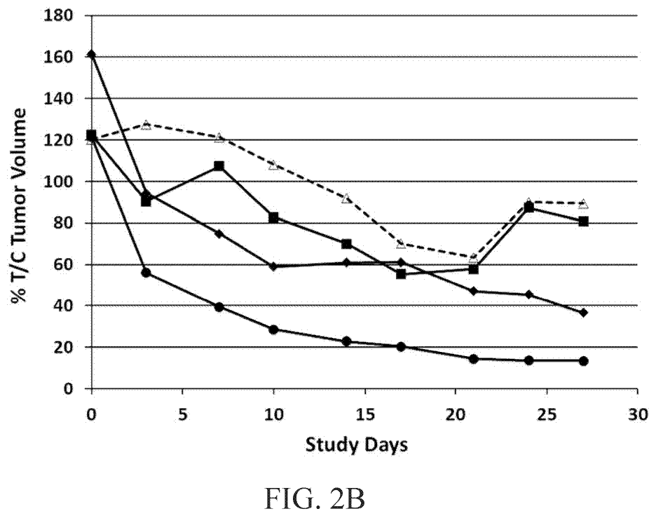

The same procedures were followed as in Example 3 with KRAS^{G12D} CRC patient NYL170-P2 using various combinations as indicated. The combination therapy scheme is shown in Table 12. The mean tumor volumes are shown in FIG. 2A and Table 13. The % T/C Tumor Volume is shown in FIG. 2B and Table 14. After 1 treatment cycle (28 days), the triplet combination of Cetuximab, 1 mg/animal, QW*3 weeks; Trametinib, 0.3 mg/kg, QD*21 days; Palbociclib, 75 mg/kg, QD*21 days showed tumor growth inhibition at TGI % of 108% and tumor regression (tumor size reduction) at REG % of 20%, whereas each of the three single agents of Cetuximab, 1 mg/animal, QW*3 weeks; Trametinib, 0.3 mg/kg, QD*21 days, and Palbociclib, 75 mg/kg, QD*21 days showed modest tumor growth inhibition at 83%, 26% and 16%, respectively (Table 12). Tumor growth inhibition %=[1−(tumor size of treatment at the end of the study−tumor size of treatment at the start of study)/(tumor size of vehicle control at the end of study-tumor size at the start of study)]*100. Tumor regression parameter is calculated as % REG=[(tumor volume of the treatment at the start of the study−tumor volume of the treatment at the end of the study)/tumor volume of the treatment at the start of the study]*100

TABLE 12
Mice groups for Control and Combination Therapy in NYL170-P2 PDX

| | | TGI % after 1 cycle of | REG % after 1 cycle of |
|---|---|---|---|
| Group | Combination Therapy Scheme | treatment (Day 21) | treatment (Day 21) |
| Group 01 | Control, 0 mg/kg, QW*3 weeks | — | — |
| Group 02 | Cetuximab, 1 mg/animal, QW*3 weeks; | 108% | 30% |
| | Trametinib, 0.3 mg/kg, QD*21 days; | | |
| | Palbociclib, 75 mg/kg, QD*21 days | | |
| Group 03 | Cetuximab, 1 mg/animal, QW*3 weeks | 76% | — |
| Group 04 | Trametinib, 0.3 mg/kg, QD*21 days | 55% | — |
| Group 05 | Palbociclib, 75 mg/kg, QD*21 days | 48% | — |

TABLE 13
Mean tumor volume (mm³) in NYL170-P2 PDX

| Day | 0 | 3 | 7 | 10 | 14 | 17 | 21 | 24 | 27 |
|---|---|---|---|---|---|---|---|---|---|
| Control | 132.09 ± | 200.94 ± | 257.60 ± | 336.68 ± | 434.73 ± | 595.61 ± | 777.11 ± | 798.14 ± | 963.40 ± |
| | 43.46 | 9.42 | 24.62 | 105.35 | 192.16 | 314.19 | 433.76 | 353.26 | 512.66 |
| Group 02 | 160.44 ± | 112.70 ± | 101.38 ± | 96.15 ± | 99.36 ± | 121.02 ± | 111.68 ± | 108.06 ± | 129.60 ± |
| | 59.73 | 42.99 | 55.56 | 69.65 | 76.43 | 83.97 | 64.67 | 68.36 | 112.23 |
| Group 03 | 213.23 | 189.39 | 193.14 | 198.28 | 265.03 | 363.61 | 366.72 | 362.87 | 352.73 |
| Group 04 | 161.78 | 181.69 | 276.39 | 278.70 | 304.52 | 329.79 | 449.10 | 695.71 | 778.53 |
| Group 05 | 158.78 | 256.15 | 312.19 | 363.79 | 399.07 | 417.50 | 492.68 | 719.14 | 860.92 |

TABLE 14
Mean tumor volume in treated mice versus
control mice (% T/C) in NYL170-P2 PDX

| Day | 0 | 3 | 7 | 10 | 14 | 17 | 21 | 24 | 27 |
|---|---|---|---|---|---|---|---|---|---|
| Group 02 | 121.46 | 56.09 | 39.36 | 28.56 | 22.86 | 20.32 | 14.37 | 13.54 | 13.45 |
| Group 03 | 161.43 | 94.25 | 74.98 | 58.89 | 60.96 | 61.05 | 47.19 | 45.46 | 36.61 |
| Group 04 | 122.48 | 90.42 | 107.30 | 82.78 | 70.05 | 55.37 | 57.79 | 87.17 | 80.81 |
| Group 05 | 120.20 | 127.48 | 121.19 | 108.05 | 91.80 | 70.10 | 63.40 | 90.10 | 89.36 |

### Example 5 Tumor Volume Reduction in Mice During Combination Therapy in a PDX Model with Patient NYL170-P3

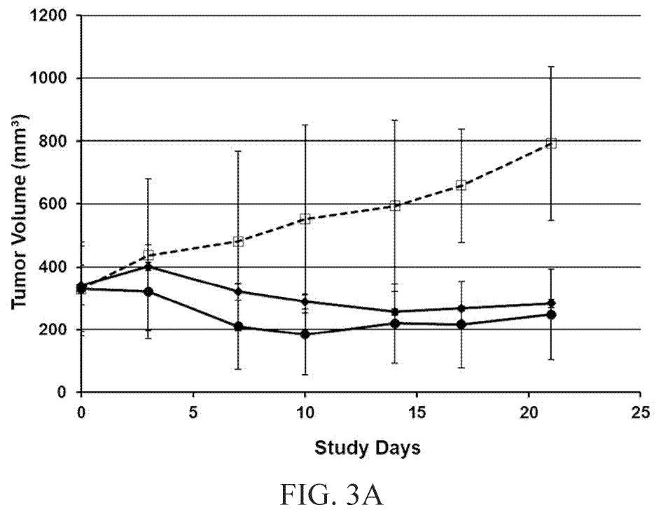

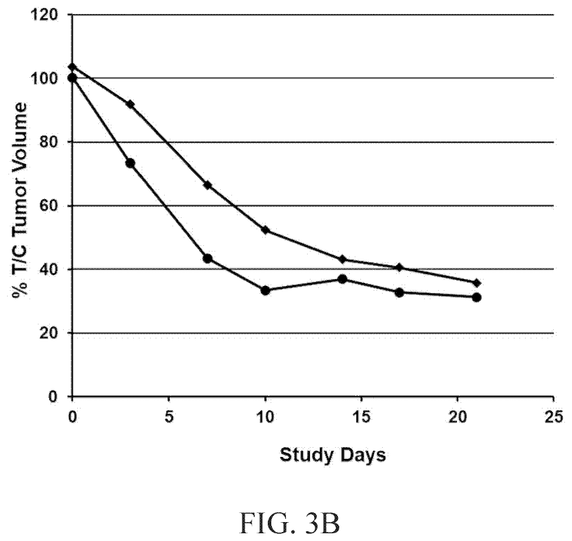

The same procedures were followed as in Example 3 with KRAS^{G12D} CRC patient NYL170-P3 using various combinations as indicated. The combination therapy scheme is shown in Table 15. The mean tumor volumes are shown in FIG. 3A and Table 16. The % T/C Tumor Volume is shown in FIG. 3B and Table 17. After 1 cycle of treatment, the triplet combination of Lapatinib, Tametinib, and Palbociclib showed tumor regression at REG % of 25% and 17%, and tumor growth inhibition (TGI %) of 118% and 113% for Group 02 and Group 03, respectively (Table 15).

TABLE 15
Mice groups for Control and Combination
Therapy in NYL170-P3 PDX

| | | TGI % after | |
|---|---|---|---|
| | | 1 Cycle of | REG |
| Group | Combination Therapy Scheme | treatment | % |
| Group 01 | Control, 0 mg/kg, QW*3 weeks | — | — |
| Group 02 | Lapatinib, 100 mg/kg, QD*21 days; | 118% | 25% |
| | Trametinib, 0.3 mg/kg, QD*21 days; | | |
| | Palbociclib, 75 mg/kg, QD*21 days | | |
| Group 03 | Lapatinib, 50 mg/kg, QD*21 days; | 113% | 17% |
| | Trametinib, 0.15 mg/kg, QD*21 days; | | |
| | Palbociclib, 37.5 mg/kg, QD*21 days | | |

TABLE 16
Mean tumor volume (mm³) in NYL170-P3 PDX

| Day | 0 | 3 | 7 | 10 | 14 | 17 | 21 |
|---|---|---|---|---|---|---|---|
| Control | 329.77 ± | 437.56 ± | 482.01 ± | 551.94 ± | 593.98 ± | 658.54 ± | 792.73 ± |
| | 151.39 | 241.74 | 285.41 | 299.54 | 272.58 | 179.85 | 244.44 |
| Group 02 | 330.61 ± | 321.43 ± | 209.48 ± | 184.32 ± | 219.17 ± | 215.86 ± | 247.50 ± |
| | 136.44 | 150.91 | 135.75 | 129.03 | 125.89 | 137.65 | 145.16 |
| Group 03 | 341.81 ± | 401.49 ± | 321.29 ± | 289.17 ± | 256.16 ± | 266.95 ± | 283.36 ± |
| | 63.75 | 12.97 | 26.95 | 22.40 | 9.59 | 8.29 | 12.69 |

TABLE 17
Mean tumor volume in treated mice versus
control mice (% T/C) in NYL170-P3 PDX

| Day | 0 | 3 | 7 | 10 | 14 | 17 | 21 |
|---|---|---|---|---|---|---|---|
| Group 02 | 100.26 | 73.46 | 43.46 | 33.39 | 36.90 | 32.78 | 31.22 |
| Group 03 | 103.65 | 91.76 | 66.66 | 52.39 | 43.13 | 40.54 | 35.74 |

### Example 6 Tumor Volume Reduction in Mice During Combination Therapy in a PDX Model with Patient NYL178-P2

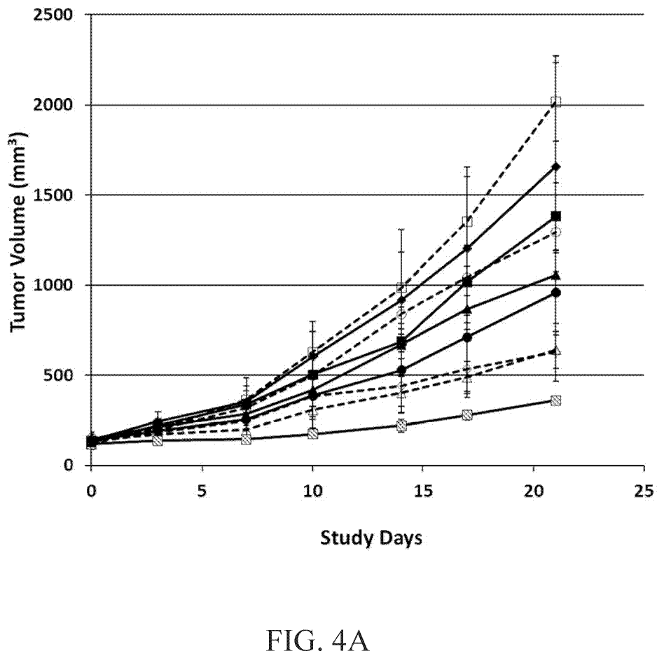

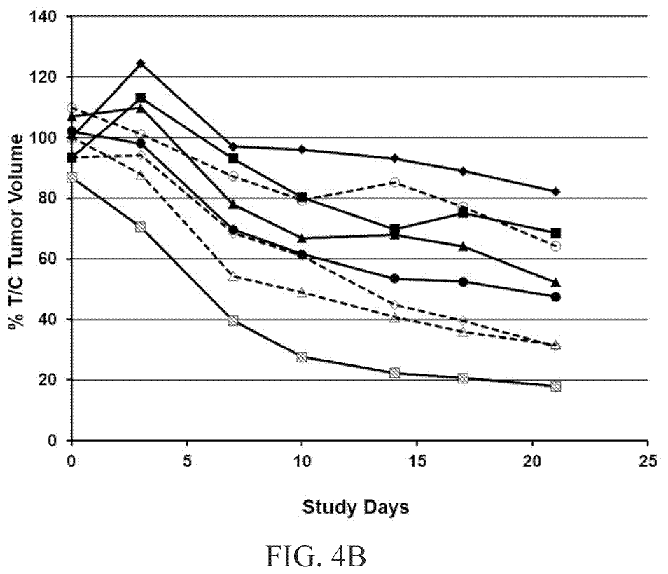

The same procedures were followed as in Example 3 with KRAS^{G13D} CRC patient NYL178-P2 using various combinations as indicated. The combination therapy scheme is shown in Table 18. The mean tumor volumes are shown in FIG. 4A and Table 19. The % TIC Tumor Volume is shown in FIG. 4B and Table 20. The triplet combination of Group 02 and 03 showed the highest tumor growth inhibition at TGI % of 83% and 87% respectively in this study. The doublet combination of Group 04, 05, 06 showed modest tumor growth inhibition at TGI % of 56%, 48% and 73%, respectively. The singlet treatment of Group 07, 08 and 09 showed the lowest TGI % at 19%, 39%, and 37%, respectively (Table 21).

TABLE 18
Mice groups for Control and Combination
Therapy in NYL178-P2 PDX

| | | TG1% after 1 cycle |
|---|---|---|
| Group | Combination Therapy Scheme | of treatment |
| Group 01 | Control, 0 mg/kg, QW*3 weeks | — |
| Group 02 | Cetuximab, 1 mg/animal, QW*3 weeks; | 83% |
| | Trametinib, 0.3 mg/kg, QD*21 days; | |
| | Palbociclib, 75 mg/kg, QD*21 days | |
| Group 03 | Lapatinib, 100 mg/kg, QD*21 days; | 87% |
| | Trametinib, 0.3 mg/kg, QD*21 days; | |
| | Palbociclib, 75 mg/kg, QD*21 days | |
| Group 04 | Cetuximab, 1 mg/animal, QW*3 weeks; | 56% |
| | Palbociclib, 75 mg/kg, QD*21 days | |
| Group 05 | Cetuximab, 1 mg/animal, QW*3 weeks; | 48% |
| | Trametinib, 0.3 mg/kg, QD*21 days | |
| Group 06 | Trametinib, 0.3 mg/kg, QD*21 days; | 73% |
| | Palbociclib, 75 mg/kg, QD*21 days | |
| Group 07 | Cetuximab, 1 mg/animal, QW*3 weeks | 19% |
| Group 08 | Trametinib, 0.3 mg/kg, QD*21 days | 39% |
| Group 09 | Palbociclib, 75 mg/kg, QD*21 days | 37% |

TABLE 19
Mean tumor volume (mm³) in NYL178-P2 PDX

| Day | 0 | 3 | 7 | 10 | 14 | 17 | 21 | 24 | 28 |
|---|---|---|---|---|---|---|---|---|---|
| Control | 136.49 ± | 195.63 ± | 363.77 ± | 627.91 ± | 984.85 ± | 1353.07 ± | 2016.85 ± | — | — |
| | 47.06 | 10.85 | 76.52 | 114.12 | 198.54 | 250.09 | 217.31 | | |
| Group 02 | 136.85 ± | 171.82 ± | 198.10 ± | 308.01 ± | 401.89 ± | 487.59 ± | 640.81 ± | 927.62 ± | — |
| | 35.93 | 46.82 | 47.87 | 100.79 | 113.90 | 89.03 | 101.94 | 179.99 | |
| Group 03 | 118.62 ± | 137.72 ± | 144.44 ± | 173.21 ± | 219.66 ± | 279.14 ± | 360.57 ± | 532.68 ± | 992.69 ± |
| | 18.93 | 6.50 | 21.40 | 3.35 | 36.27 | 26.79 | 24.57 | 59.10 | 212.39 |
| Group 04 | 139.34 ± | 191.88 ± | 253.29 ± | 386.56 ± | 527.10 ± | 711.08 ± | 958.60 ± | — | — |
| | 15.90 | 67.70 | 96.30 | 131.13 | 199.21 | 302.59 | 236.95 | | |
| Group 05 | 146.12 ± | 214.91 ± | 283.98 ± | 419.32 ± | 669.10 ± | 867.75 ± | 1056.43 ± | — | — |
| | 26.22 | 45.66 | 6.08 | 93.74 | 40.37 | 75.25 | 17.49 | | |
| Group 06 | 127.54 ± | 184.45 ± | 249.23 ± | 381.93 ± | 441.80 ± | 535.02 ± | 628.56 ± | 751.37 ± | 1129.60 ± |
| | 20.48 | 63.54 | 59.14 | 111.52 | 149.76 | 157.93 | 160.32 | 172.51 | 253.37 |
| Group 07 | 136.65 ± | 243.46 ± | 353.00 ± | 603.08 ± | 917.09 ± | 1203.07 ± | 1658.38 ± | — | — |
| | 37.27 | 51.93 | 133.25 | 194.60 | 390.50 | 451.10 | 610.94 | | |
| Group 08 | 149.92 ± | 197.90 ± | 317.54 ± | 498.25 ± | 839.27 ± | 1043.54 ± | 1294.21 ± | — | — |
| | 20.97 | 13.82 | 23.94 | 13.08 | 80.77 | 177.20 | 113.61 | | |
| Group 09 | 127.47 ± | 221.27 ± | 338.59 ± | 504.20 ± | 686.82 ± | 1016.88 ± | 1380.44 ± | — | — |
| | 21.27 | 24.53 | 76.99 | 113.44 | 192.90 | 182.51 | 21.27 | | |

TABLE 20
Mean tumor volume in treated mice versus
control mice (% T/C) in NYL178-P2 PDX

| Day | 0 | 3 | 7 | 10 | 14 | 17 | 21 | 24 | 28 |
|---|---|---|---|---|---|---|---|---|---|
| Group | 100.27 | 87.83 | 54.46 | 49.05 | 40.81 | 36.04 | 31.77 | — | — |
| 02 | | | | | | | | | |
| Group | 86.91 | 70.40 | 39.71 | 27.58 | 22.30 | 20.63 | 17.88 | — | — |
| 03 | | | | | | | | | |
| Group | 102.09 | 98.09 | 69.63 | 61.56 | 53.52 | 52.55 | 47.53 | — | — |
| 04 | | | | | | | | | |
| Group | 107.06 | 109.86 | 78.07 | 66.78 | 67.94 | 64.13 | 52.38 | — | — |
| 05 | | | | | | | | | |
| Group | 93.45 | 94.29 | 68.51 | 60.83 | 44.86 | 39.54 | 31.17 | — | — |
| 06 | | | | | | | | | |
| Group | 100.12 | 124.45 | 97.04 | 96.05 | 93.12 | 88.91 | 82.23 | — | — |
| 07 | | | | | | | | | |
| Group | 109.84 | 101.16 | 87.29 | 79.35 | 85.22 | 77.12 | 64.17 | — | — |
| 08 | | | | | | | | | |
| Group | 93.39 | 113.11 | 93.08 | 80.30 | 69.74 | 75.15 | 68.45 | — | — |
| 09 | | | | | | | | | |

TABLE 21
Tumor Growth Inhibition (TGI %) in NYL178-P2 PDX

| | Tumor volume | Tumor volume | TGI % after 1 cycle |
|---|---|---|---|
| Group | at Day 0 (mm³) | at Day 21 (mm³) | of treatment |
| Control | 136.49 ± 47.06 | 2016.85 ± 217.31 | — |
| Group 02 | 136.85 ± 35.93 | 640.81 ± 101.94 | 83% |
| Group 03 | 118.62 ± 18.93 | 360.57 ± 24.57 | 87% |
| Group 04 | 139.34 ± 15.90 | 958.60 ± 236.95 | 56% |
| Group 05 | 146.12 ± 26.22 | 1056.43 ± 17.49 | 48% |
| Group 06 | 127.54 ± 20.48 | 628.56 ± 160.32 | 73% |
| Group 07 | 136.65 ± 37.27 | 1658.38 ± 610.94 | 19% |
| Group 08 | 149.92 ± 20.97 | 1294.21 ± 113.61 | 39% |
| Group 09 | 127.47 ± 21.27 | 1380.44 ± 21.27 | 37% |

### Example 7 Tumor Volume Reduction in Mice during Combination Therapy in a PDX Model with Patient NYL178-P4

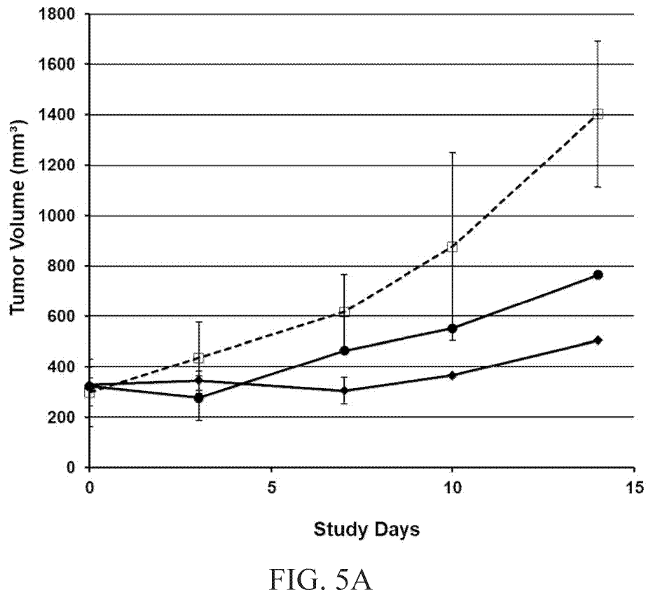

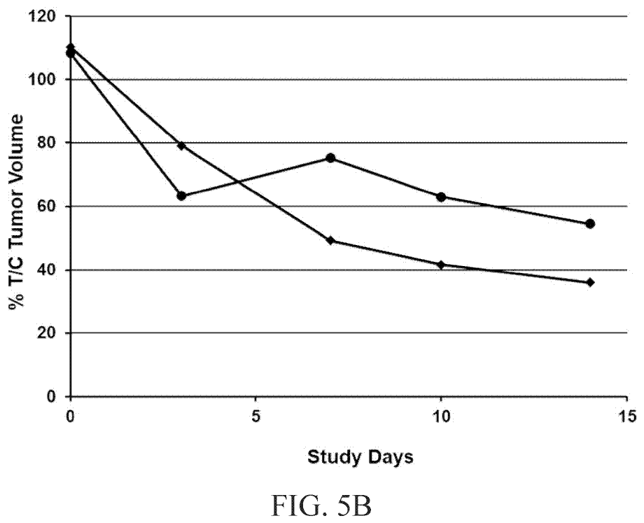

The same procedures were followed as in Example 3 with KRAS^{G13D} CRC patient NYL178-P4 using various combinations as indicated. The combination therapy scheme is shown in Table 22. The mean tumor volumes are shown in FIG. 5A and Table 23. The % T/C Tumor Volume is shown in FIG. 5B and Table 24. The combination treatments in Group 02 and Group 03 showed TGI % at 60% and 84%, respectively, after 14 days of treatment (Table 25).

TABLE 22
Mice groups for Control and Combination
Therapy in NYL178-P4 PDX

| | | TGI % after |
|---|---|---|
| | | 14 Days of |
| Group | Combination Therapy Scheme | treatment |
| Group | Control, 0 mg/kg, QW*3 weeks | — |
| 01 | | |
| Group | Lapatinib, 100 mg/kg, QD*14 days; Cetuximab, | 60% |
| 02 | 1 mg/animal, QW*2 weeks; Palbociclib, | |
| | 75 mg/kg, QD*14 days | |
| Group | Lapatinib, 100 mg/kg, QD*14 days; Trametinib, | 84% |
| 03 | 0.3 mg/kg, QD*14 days; Palbociclib, | |
| | 75 mg/kg, QD*14 days | |

TABLE 23
Mean tumor volume (mm³) in NYL178-P4 PDX

| Day | 0 | 3 | 7 | 10 | 14 |
|---|---|---|---|---|---|
| Control | 297.09 ± | 435.52 ± | 616.53 ± | 876.88 ± | 1402.98 ± |
| | 133.65 | 141.42 | 149.26 | 373.71 | 289.09 |
| Group 02 | 321.89 ± | 275.60 ± | 464.02 | 551.99 | 764.31 |
| | 77.02 | 87.02 | | | |
| Group 03 | 327.55 ± | 344.52 ± | 304.01 ± | 365.11 | 504.22 |
| | 26.17 | 38.59 | 52.82 | | |

TABLE 24
Mean tumor volume in treated mice versus
control mice (% T/C) in NYL178-P4 PDX

| Day | 0 | 3 | 7 | 10 | 14 |
|---|---|---|---|---|---|
| Group 02 | 108.35 | 63.28 | 75.26 | 62.95 | 54.48 |
| Group 03 | 110.25 | 79.11 | 49.31 | 41.64 | 35.94 |

TABLE 25
Tumor Growth Inhibition (TGI %) in NYL178-P4 PDX

| | Tumor volume at | Tumor volume at | TGI % after 14 days |
|---|---|---|---|
| Group | Day 0 (mm³) | Day 14 (mm³) | of treatment |
| Control | 297.09 ± 133.65 | 1402.98 ± 289.09 | — |
| Group 02 | 321.89 ± 77.02 | 764.31 | 60% |
| Group 03 | 327.55 ± 26.17 | 504.22 | 84% |

### Example 8 Tumor Volume Reduction in Mice During Combination Therapy in a PDX Model with Patient NYL-JN-025

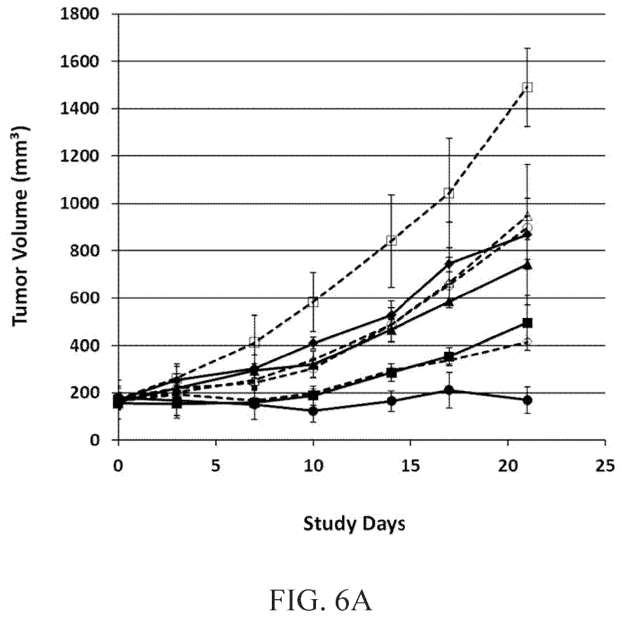

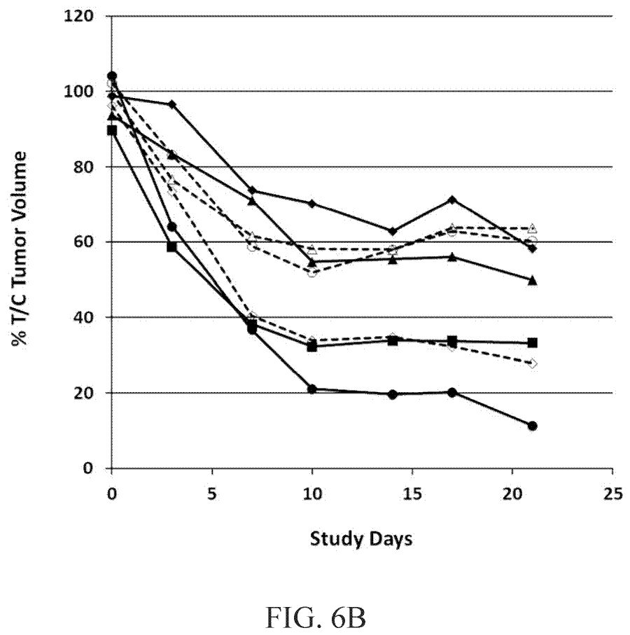

The same procedures were followed as in Example 3 with KRAS^{G12D} CRC patient NYL-JN-025 using various combinations as indicated. The combination therapy scheme is shown in Table 26. The mean tumor volumes are shown in FIG. 6A and Table 27. The % T/C Tumor Volume is shown in FIG. 6B and Table 28. After 1 cycle of treatment, triplet combination of Group 02 showed complete tumor growth inhibition (TGI % at 101%), which is better than any of the doublet combinations in Group 03, 04, 05 (TGI % at 81%, 74%, 56%, respectively), and any of the singlet treatment in Group 06, 07 and 08 (TGI % at 41%, 45% and 47% respectively) (Table 26).

TABLE 26
Mice groups for Control and Combination
Therapy in NYL-JN-025 PDX

| | | TGI % After |
|---|---|---|
| | | 1 Cycle of |
| Group | Combination Therapy Scheme | Treatment |
| Group 01 | Control, 0 mg/kg, QD*14 days | — |
| Group 02 | Cetuximab, 1 mg/animal, QW*3 weeks; | 101% |
| | Trametinib, 0.3 mg/kg, QD*21 days; | |
| | Palbociclib, 75 mg/kg, QD*21 days | |
| Group 03 | Cetuximab, 1 mg/animal, QW*3 weeks; | 81% |
| | Palbociclib, 75 mg/kg, QD*21 days | |
| Group 04 | Cetuximab, 1 mg/animal, QW*3 weeks; | 74% |
| | Trametinib, 0.3 mg/kg, QD*21 days | |
| Group 05 | Trametinib, 0.3 mg/kg, QD*21 days; | 56% |
| | Palbociclib, 75 mg/kg, QD*21 days | |
| Group 06 | Cetuximab, 1 mg/animal, QW*3 weeks | 41% |
| Group 07 | Trametinib, 0.3 mg/kg, QD*21 days | 45% |
| Group 08 | Palbociclib, 75 mg/kg, QD*21 days | 47% |

TABLE 27
Mean tumor volume (mm³) in NYL-JN-025 PDX

| Day | 0 | 3 | 7 | 10 | 14 | 17 | 21 |
|---|---|---|---|---|---|---|---|
| Control | 172.23 ± | 261.91 ± | 412.36 ± | 584.11 ± | 841.31 ± | 1044.77 ± | 1490.04 ± |
| | 30.35 | 61.61 | 115.53 | 125.22 | 195.24 | 231.90 | 166.53 |
| Group 02 | 179.41 ± | 167.85 ± | 151.27 ± | 123.23 ± | 165.10 ± | 210.60 ± | 168.81 ± |
| | 48.85 | 63.49 | 65.85 | 47.83 | 42.52 | 74.00 | 56.78 |
| Group 03 | 165.66 ± | 192.42 ± | 166.99 ± | 197.80 ± | 292.66 ± | 336.84 ± | 415.47 ± |
| | 19.87 | 34.78 | 12.93 | 18.24 | 7.50 | 16.94 | 12.31 |
| Group 04 | 154.47 ± | 153.41 ± | 157.51 ± | 188.65 ± | 285.19 ± | 353.36 ± | 494.85 ± |
| | 27.53 | 1.37 | 5.86 | 40.02 | 36.86 | 39.39 | 115.63 |
| Group 05 | 161.28 ± | 218.39 ± | 293.34 ± | 319.39 ± | 465.76 ± | 585.21 ± | 742.91 ± |
| | 8.26 | 2.59 | 66.32 | 57.51 | 47.29 | 0.16 | 20.10 |
| Group 06 | 171.83 ± | 200.71 ± | 253.58 ± | 339.42 ± | 487.54 ± | 665.59 ± | 948.04 ± |
| | 83.01 | 109.11 | 43.08 | 2.32 | 72.51 | 106.83 | 74.75 |
| Group 07 | 175.95 ± | 217.82 ± | 242.66 ± | 302.87 ± | 487.05 ± | 656.21 ± | 895.96 ± |
| | 42.43 | 28.44 | 20.19 | 38.13 | 22.52 | 55.96 | 50.35 |
| Group 08 | 170.12 ± | 252.65 ± | 303.80 ± | 409.77 ± | 528.04 ± | 744.41 ± | 867.00 ± |
| | 28.56 | 28.21 | 19.06 | 24.21 | 58.23 | 177.02 | 295.40 |

TABLE 28
Mean tumor volume in treated mice versus
control mice (% T/C) in NYL-JN-025 PDX

| Day | 0 | 3 | 7 | 10 | 14 | 17 | 21 |
|---|---|---|---|---|---|---|---|
| Group 02 | 104.17 | 64.09 | 36.68 | 21.10 | 19.62 | 20.16 | 11.33 |
| Group 03 | 96.18 | 73.47 | 40.49 | 33.86 | 34.79 | 32.24 | 27.88 |
| Group 04 | 89.68 | 58.57 | 38.20 | 32.30 | 33.90 | 33.82 | 33.21 |
| Group 05 | 93.64 | 83.38 | 71.14 | 54.68 | 55.36 | 56.01 | 49.86 |
| Group 06 | 99.77 | 76.63 | 61.49 | 58.11 | 57.95 | 63.71 | 63.63 |
| Group 07 | 102.16 | 83.17 | 58.85 | 51.85 | 57.89 | 62.81 | 60.13 |
| Group 08 | 98.77 | 96.46 | 73.67 | 70.15 | 62.76 | 71.25 | 58.19 |

### Example 9 Tumor Volume Reduction in Mice During Combination Therapy in a PDX Model with Patient NYP031-P8

The same procedures were followed as in Example 3 with KRAS^{SNV:c.G35T:p.G12V} CRC patient NYP031-P8 using various combinations as indicated. The combination therapy scheme is shown in Table 29. The mean tumor volumes are shown in Table 30. The % TIC Tumor Volume is shown in Table 31. The triplet combination of Group 06 showed strong growth inhibition at TGI % of 95%, which is higher than any doublet combination or singlet treatment, as well as higher than combination of chemotherapeutics agents. Furthermore, the tumor growth inhibition rate is comparable to that with known chemotherapeutic+antibody combinations such as the BCapOx regimen in Group 13, and the CAPIRI regimen in Group 14, (with TGI % at 105% and 98% respectively) (Table 29).

TABLE 29
Mice groups for Control and Combination
Therapy in NYP031-P8 PDX

| | | TGI % After |
|---|---|---|
| | | 1 Cycle of |
| Group | Combination Therapy Scheme | Treatment |
| Group 01 | Control, 0 mg/kg, QD*3 weeks | — |
| Group 02 | Capecitabine, 200 mg/kg, QD*21 days | 50% |
| Group 03 | Capecitabine, 200 mg/kg, QD*21 days; | 57% |
| | Oxaliplatin, 10 mg/kg, QW*3 weeks | |
| Group 04 | Capecitabine, 200 mg/kg, QD*21 days; | 87% |
| | Irinotecan, 100 mg/kg, QW*3 weeks | |
| Group 05 | Docetaxel, 10 mg/kg, QW*3 weeks | 56% |
| Group 06 | Cetuximab, 1 mg/animal, QW*3 weeks; | 95% |
| | Trametinib, 0.3 mg/kg, QD*21 days; | |
| | Palbociclib, 75 mg/kg, QD*21 days | |
| Group 07 | Cetuximab, 1 mg/animal, QW*3 weeks; | 73% |
| | Trametinib, 0.3 mg/kg, QD*21 days | |
| Group 08 | Cetuximab, 1 mg/animal, QW*3 weeks; | 62% |
| | Palbociclib, 75 mg/kg, QD*21 days | |
| Group 09 | Trametinib, 0.3 mg/kg, QD*21 days; | 70% |
| | Palbociclib, 75 mg/kg, QD*21 days | |
| Group 10 | Cetuximab, 1 mg/animal, QW*3 weeks | 56% |
| Group 11 | Trametinib, 0.3 mg/kg, QD*21 days | 34% |
| Group 12 | Palbociclib, 75 mg/kg, QD*21 days | 30% |
| Group 13 | Oxaliplatin, 10 mg/kg, QW*3 weeks; | 105% |
| | Capecitabine, 200 mg/kg, QD*21 days; | |
| | Bevacizumab, 10 mg/kg, QW*3 weeks | |
| Group 14 | Capecitabine, 200 mg/kg, QD*21 days; | 98% |
| | Irinotecan, 100 mg/kg, QW*3 weeks; | |
| | Bevacizumab, 10 mg/kg, QW*3 weeks | |

TABLE 30
Mean tumor volume (mm³) in NYP031-P8 PDX

| Day | 0 | 3 | 7 | 10 | 14 | 17 | 21 |
|---|---|---|---|---|---|---|---|
| Control | 293.46 ± | 467.49 ± | 642.18 ± | 814.24 ± | 993.76 ± | 1157.78 ± | 1486.25 ± |
| | 70.39 | 140.19 | 140.57 | 119.32 | 125.29 | 29.75 | 182.22 |
| Group 02 | 289.87 ± | 350.23 ± | 397.71 ± | 399.35 ± | 526.12 ± | 670.67 ± | 886.72 ± |
| | 61.75 | 50.85 | 84.48 | 103.65 | 206.07 | 244.84 | 191.23 |
| Group 03 | 296.25 ± | 341.68 ± | 420.11 ± | 442.15 ± | 556.00 ± | 668.59 ± | 803.61 ± |
| | 68.66 | 83.43 | 155.83 | 205.87 | 237.67 | 270.10 | 286.93 |
| Group 04 | 289.87 ± | 422.42 ± | 476.04 ± | 456.88 ± | 498.31 ± | 475.25 ± | 442.37 ± |
| | 57.61 | 71.56 | 119.83 | 76.65 | 36.29 | 43.29 | 36.97 |
| Group 05 | 303.03 ± | 396.64 ± | 478.86 ± | 576.46 ± | 740.73 ± | 767.12 ± | 826.48 ± |
| | 16.63 | 95.89 | 163.95 | 197.89 | 257.84 | 210.86 | 297.65 |
| Group 06 | 289.69 ± | 276.06 ± | 235.68 ± | 238.98 ± | 288.44 ± | 326.17 ± | 353.35 ± |
| | 52.32 | 45.17 | 23.89 | 30.73 | 17.60 | 22.62 | 30.08 |
| Group 07 | 289.22 ± | 299.90 ± | 311.91 ± | 345.85 ± | 482.30 ± | 539.48 ± | 612.23 ± |
| | 91.88 | 130.39 | 150.47 | 155.85 | 232.02 | 245.73 | 360.72 |
| Group 08 | 305.26 ± | 366.07 ± | 369.78 ± | 402.59 ± | 486.70 ± | 623.52 ± | 760.92 ± |
| | 82.89 | 98.66 | 107.12 | 93.19 | 147.70 | 166.65 | 211.35 |
| Group 09 | 301.36 ± | 449.91 ± | 396.46 ± | 408.00 ± | 588.39 ± | 590.35 ± | 662.74 ± |
| | 69.69 | 125.79 | 131.26 | 138.69 | 205.15 | 206.18 | 186.53 |
| Group 10 | 303.85 ± | 354.50 ± | 387.17 ± | 463.79 ± | 583.74 ± | 643.53 ± | 829.94 ± |
| | 72.60 | 146.74 | 124.69 | 91.89 | 137.01 | 114.64 | 124.74 |
| Group 11 | 300.84 ± | 416.34 ± | 500.82 ± | 654.63 ± | 888.43 ± | 956.93 ± | 1086.50 ± |
| | 64.94 | 32.34 | 31.63 | 56.47 | 125.95 | 80.70 | 106.98 |
| Group 12 | 297.31 ± | 450.85 ± | 565.02 ± | 697.27 ± | 969.29 ± | 1052.09 ± | 1134.97 ± |
| | 57.31 | 69.43 | 89.35 | 161.33 | 233.05 | 206.30 | 146.43 |
| Group 13 | 296.27 ± | 346.44 ± | 355.99 ± | 326.73 ± | 265.29 ± | 270.98 ± | 241.24 ± |
| | 53.37 | 106.09 | 73.06 | 61.03 | 49.20 | 41.20 | 26.88 |
| Group 14 | 301.52 ± | 362.39 ± | 359.90 ± | 322.84 ± | 365.45 ± | 354.47 ± | 326.83 ± |
| | 80.41 | 126.70 | 106.60 | 70.41 | 151.11 | 148.38 | 140.27 |

TABLE 31
Mean tumor volume in treated mice versus
control mice (% T/C) in NYP031-P8 PDX

| Day | 0 | 3 | 7 | 10 | 14 | 17 | 21 |
|---|---|---|---|---|---|---|---|
| Group 02 | 98.78 | 74.92 | 61.93 | 49.05 | 52.94 | 57.93 | 59.66 |
| Group 03 | 100.95 | 73.09 | 65.42 | 54.30 | 55.95 | 57.75 | 54.07 |
| Group 04 | 98.78 | 90.36 | 74.13 | 56.11 | 50.14 | 41.05 | 29.76 |
| Group 05 | 103.26 | 84.84 | 74.57 | 70.80 | 74.54 | 66.26 | 55.61 |
| Group 06 | 98.72 | 59.05 | 36.70 | 29.35 | 29.03 | 28.17 | 23.77 |
| Group 07 | 98.55 | 64.15 | 48.57 | 42.48 | 48.53 | 46.60 | 41.19 |
| Group 08 | 104.02 | 78.31 | 57.58 | 49.44 | 48.98 | 53.86 | 51.20 |
| Group 09 | 102.69 | 96.24 | 61.74 | 50.11 | 59.21 | 50.99 | 44.59 |
| Group 10 | 103.54 | 75.83 | 60.29 | 56.96 | 58.74 | 55.58 | 55.84 |
| Group 11 | 102.52 | 89.06 | 77.99 | 80.40 | 89.40 | 82.65 | 73.10 |
| Group 12 | 101.31 | 96.44 | 87.99 | 85.63 | 97.54 | 90.87 | 76.36 |
| Group 13 | 100.96 | 74.11 | 55.43 | 40.13 | 26.70 | 23.41 | 16.23 |
| Group 14 | 102.75 | 77.52 | 56.04 | 39.65 | 36.77 | 30.62 | 21.99 |

### Example 10 Tumor Volume Reduction in Mice during Combination Therapy in a PDX Model with Patient NYP031-P9

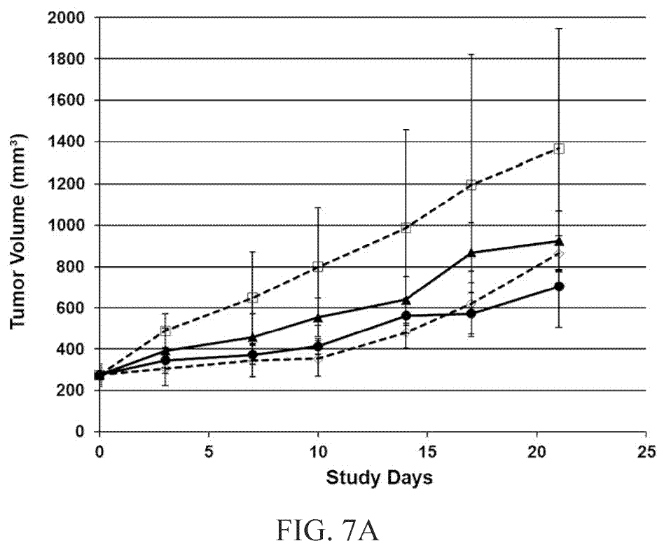

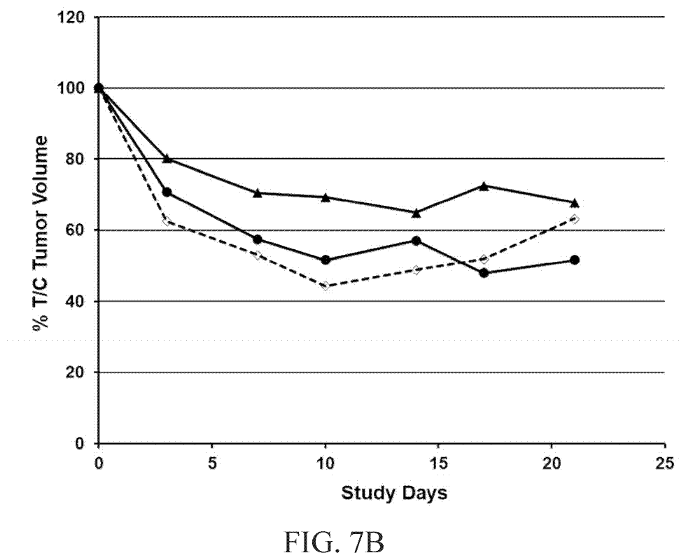

The same procedures were followed as in Example 3 with KRAS^{G12V} CRC patient NYP031-P9 using various combinations as indicated. The combination therapy scheme is shown in Table 32. The mean tumor volumes are shown in FIG. 7A and Table 33. The % T/C Tumor Volume is shown in FIG. 7B and Table 34. After 21 days (one cycle of treatment), the triplet combination treatment of cobimetinib, osimertinib and palbociclib showed cobimetinib-concentration dependent growth inhibition (TGI % at 61%, 46% and 40% for Group 02, 03 and 04, respectively) (Table 32).

TABLE 32
Mice groups for Control and Combination
Therapy in NYP031-P9 PDX

| | | TGI % After |
|---|---|---|
| | | 1 Cycle of |
| Group | Combination Therapy Scheme | Treatment |
| Group 01 | Control, 0 mg/kg, QD*3 weeks | — |
| Group 02 | Cobimetinib, 5.2 mg/kg, QD*21 days; | 61% |
| | Osimertinib, 10 mg/kg, QD*21 days; | |
| | Palbociclib, 75 mg/kg, QD*21 days | |
| Group 03 | Palbociclib, 75 mg/kg, QD*21 days; | 46% |
| | Osimertinib, 10 mg/kg, QD*21 days; | |
| | Cobimetinib, 2.6 mg/kg, QD*21 days | |
| Group 04 | Cobimetinib, 1.3 mg/kg, QD*21 days; | 40% |
| | Osmertinib, 10 mg/kg, QD*21 days; | |
| | Palbociclib, 75 mg/kg, QD*21 days | |

TABLE 33
Mean tumor volume (mm³) in NYP031-P9 PDX

| Day | 0 | 3 | 7 | 10 | 14 | 17 | 21 |
|---|---|---|---|---|---|---|---|
| Control | 274.44 ± | 489.64 ± | 648.05 ± | 799.86 ± | 986.00 ± | 1195.84 ± | 1365.50 ± |
| | 24.71 | 82.00 | 222.03 | 284.36 | 471.70 | 625.37 | 579.75 |
| Group 02 | 274.64 ± | 346.03 ± | 371.79 ± | 412.58 ± | 562.36 ± | 573.18 ± | 704.56 ± |
| | 34.51 | 35.87 | 44.86 | 39.14 | 85.29 | 98.97 | 199.42 |
| Group 03 | 274.91 ± | 305.88 ± | 343.68 ± | 354.37 ± | 481.21 ± | 619.88 ± | 863.00 ± |
| | 44.26 | 81.54 | 77.08 | 85.17 | 78.54 | 159.72 | 87.75 |
| Group 04 | 274.58 ± | 392.11 ± | 456.78 ± | 554.07 ± | 639.28 ± | 867.89 ± | 924.51 ± |
| | 54.00 | 109.46 | 113.41 | 93.36 | 113.88 | 144.61 | 142.02 |

TABLE 34
Mean tumor volume in treated mice versus
control mice (% T/C) in NYP031-P9 PDX

| Day | 0 | 3 | 7 | 10 | 14 | 17 | 21 |
|---|---|---|---|---|---|---|---|
| Group 02 | 100.07 | 70.67 | 57.37 | 51.58 | 57.03 | 47.93 | 51.60 |
| Group 03 | 100.17 | 62.47 | 53.03 | 44.30 | 48.80 | 51.84 | 63.20 |
| Group 04 | 100.05 | 80.08 | 70.49 | 69.27 | 64.84 | 72.58 | 67.70 |

### Example 11 Tumor Volume Reduction in Mice during Combination Therapy in a PDX Model with Patient ZKB171-P2

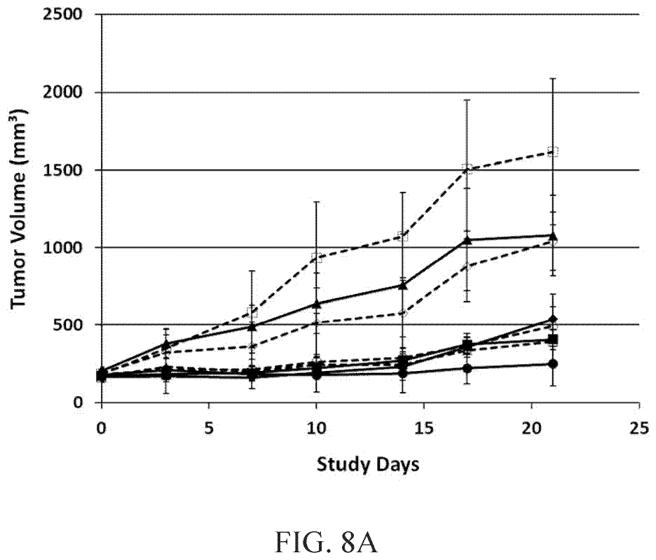

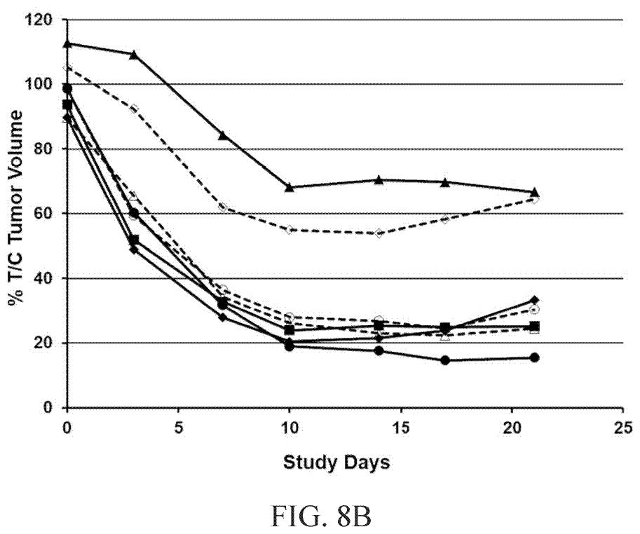

The same procedures were followed as in Example 3 with KRAS^{G12D} CRC patient ZKB171 using various combinations as indicated. The combination therapy scheme is shown in Table 35. The mean tumor volumes are shown in FIG. 8A and Table 36. The % T/C Tumor Volume is shown in FIG. 7B and Table 37. After 21 days (one cycle of treatment), the triplet combination treatment (Group 02) showed the highest growth inhibition (TGI % 95%), as compared to the doublet combination (Group 03, 04, 05, TGI %=78%, 84%, 84%, respectively) and singlet treatment (Group 06, 07, 08, TGI %=74%, 3=41%, 39%, respectively) treatments (Table 38).

TABLE 35
Mice groups for Control and Combination
Therapy in ZKB171-P2 PDX

| | | TG1% After |
|---|---|---|
| | | 1 Cycle of |
| Group | Combination Therapy Scheme | Treatment |
| Group 01 | Control, 0 mg/kg, QD*14 days | — |
| Group 02 | Cetuximab, 1 mg/animal, QW*3 weeks; | 95% |
| | Trametinib, 0.3 mg/kg, QD*21 days; | |
| | Palbociclib, 75 mg/kg, QD*21 days | |
| Group 03 | Cetuximab, 1 mg/animal, QW*3 weeks; | 78% |
| | Palbociclib, 75 mg/kg, QD*21 days | |
| Group 04 | Cetuximab, 1 mg/animal, QW*3 weeks; | 84% |
| | Trametinib, 0.3 mg/kg, QD*21 days | |
| Group 05 | Trametinib, 0.3 mg/kg, QD*21 days; | 84% |
| | Palbociclib, 75 mg/kg, QD*21 days | |
| Group 06 | Cetuximab, 1 mg/animal, QW*3 weeks | 74% |
| Group 07 | Trametinib, 0.3 mg/kg, QD*21 days | 41% |
| Group 08 | Palbociclib, 75 mg/kg, QD*21 days | 39% |

TABLE 36
Mean tumor volume (mm³) in ZKB171-P2 PDX

| Day | 0 | 3 | 7 | 10 | 14 | 17 | 21 |
|---|---|---|---|---|---|---|---|
| Control | 181.98 ± | 348.03 ± | 581.45 ± | 934.27 ± | 1071.92 ± | 1506.32 ± | 1614.59 ± |
| | 44.53 | 123.81 | 264.80 | 358.62 | 285.40 | 447.02 | 470.91 |
| Group 02 | 179.55 ± | 209.88 ± | 183.83 ± | 176.99 ± | 188.25 ± | 219.67 ± | 248.86 ± |
| | 51.12 | 151.25 | 95.90 | 110.82 | 125.71 | 99.27 | 143.29 |
| Group 03 | 179.77 ± | 206.61 ± | 212.22 ± | 261.14 ± | 286.96 ± | 366.42 ± | 490.79 ± |
| | 29.06 | 15.35 | 2.50 | 34.98 | 38.04 | 56.77 | 129.99 |
| Group 04 | 170.44 ± | 180.62 ± | 190.57 ± | 222.68 ± | 271.34 ± | 373.08 ± | 405.36 ± |
| | 2.75 | 45.17 | 48.43 | 66.07 | 3.76 | 11.19 | 66.18 |
| Group 05 | 163.47 ± | 228.39 ± | 199.27 ± | 242.97 ± | 244.98 ± | 335.32 ± | 394.07 ± |
| | 4.15 | 58.67 | 31.87 | 64.32 | 102.87 | 110.05 | 147.32 |
| Group 06 | 163.40 ± | 170.37 ± | 162.13 ± | 190.75 ± | 229.65 ± | 356.88 ± | 538.23 ± |
| | 2.89 | 25.17 | 23.29 | 21.36 | 26.68 | 63.56 | 159.11 |
| Group 07 | 191.32 ± | 321.84 ± | 360.39 ± | 514.21 ± | 577.59 ± | 878.13 ± | 1039.92 ± |
| | 31.59 | 113.23 | 160.58 | 221.92 | 224.58 | 229.05 | 189.88 |
| Group 08 | 204.96 ± | 379.76 ± | 490.31 ± | 637.94 ± | 756.50 ± | 1051.12 ± | 1077.83 ± |
| | 12.75 | 97.51 | 138.40 | 194.83 | 333.67 | 331.43 | 258.82 |

TABLE 37
Mean tumor volume in treated mice versus
control mice (% T/C) in ZKB171-P2 PDX

| Day | 0 | 3 | 7 | 10 | 14 | 17 | 21 |
|---|---|---|---|---|---|---|---|
| Group 02 | 98.66 | 60.31 | 31.62 | 18.94 | 17.56 | 14.58 | 15.41 |
| Group 03 | 98.79 | 59.36 | 36.50 | 27.95 | 26.77 | 24.33 | 30.40 |
| Group 04 | 93.66 | 51.90 | 32.78 | 23.83 | 25.31 | 24.77 | 25.11 |
| Group 05 | 89.83 | 65.62 | 34.27 | 26.01 | 22.85 | 22.26 | 24.41 |
| Group 06 | 89.79 | 48.95 | 27.88 | 20.42 | 21.42 | 23.69 | 33.34 |
| Group 07 | 105.13 | 92.48 | 61.98 | 55.04 | 53.88 | 58.30 | 64.41 |
| Group 08 | 112.62 | 109.12 | 84.33 | 68.28 | 70.57 | 69.78 | 66.76 |

TABLE 38
Tumor Growth Inhibition (TGI %) in ZKB182-P2 PDX

| | Tumor volume at | Tumor volume at | |
|---|---|---|---|
| Group | Day 0 (mm³) | Day 21 (mm³) | TGI % |
| Control | 181.98 ± 44.53 | 1614.59 ± 470.91 | — |
| Group 02 | 179.55 ± 51.12 | 248.86 ± 143.29 | 95% |
| Group 03 | 179.77 ± 29.06 | 490.79 ± 129.99 | 78% |
| Group 04 | 170.44 ± 2.75 | 405.36 ± 66.18 | 84% |
| Group 05 | 163.47 ± 4.15 | 394.07 ± 147.32 | 84% |
| Group 06 | 163.40 ± 2.89 | 538.23 ± 159.11 | 74% |
| Group 07 | 191.32 ± 31.59 | 1039.92 ± 189.88 | 41% |
| Group 08 | 204.96 ± 12.75 | 1077.83 ± 258.82 | 39% |

### Example 12 Tumor Volume Reduction in Mice during Combination Therapy in a PDX Model with Patient ZKB182-P2

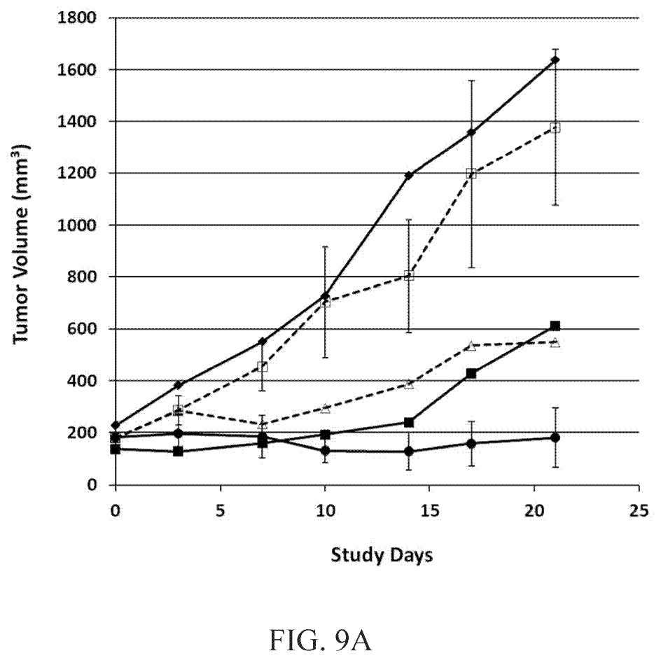

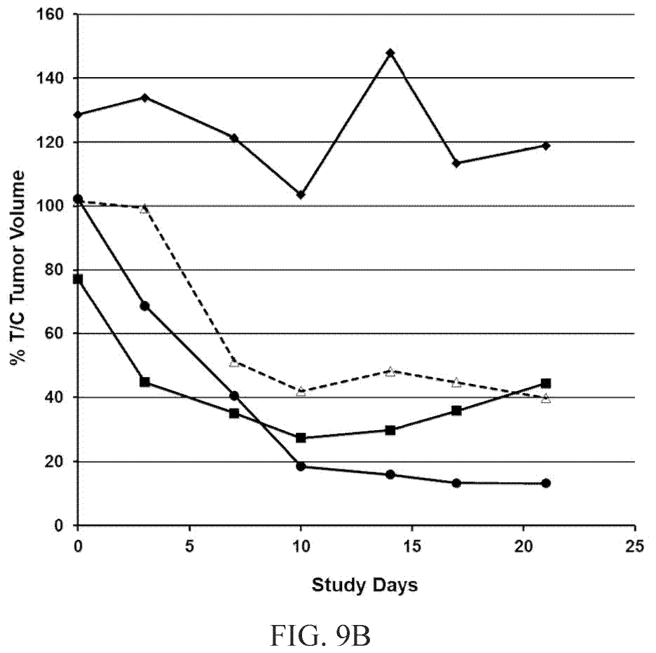

The same procedures were followed as in Example 3 with KRAS^{G12D} CRC patient ZKB182 using various combinations as indicated. The combination therapy scheme is shown in Table 39. The mean tumor volumes are shown in FIG. 9A and Table 40. The % TIC Tumor Volume is shown in FIG. 9B and Table 41. At Day 21, the triplet combination of Group 02 showed 100 growth inhibition (TGI % at 100%), whereas the singlet treatment of Group 03, 04 and 05 resulted in TGI % of −18%, 61% and 69%, respectively, In fact, Group 03 appeared to have no effect on tumor inhibition, as shown by a faster tumor growth rate than the control (Table 42).

TABLE 39
Mice groups for Control and Combination
Therapy in ZKB182-P2 PDX

| | | TGI % After |
|---|---|---|
| | | 1 Cycle of |
| Group | Combination Therapy Scheme | Treatment |
| Group 01 | Control, 0 mg/kg, QW*3 weeks | — |
| Group 02 | Lapatinib, 100 mg/kg, QD*21 days; | 100% |
| | Trametinib, 0.3 mg/kg, QD*21 days; | |
| | Palbociclib, 75 mg/kg, QD*21 days | |
| Group 03 | Lapatinib, 100 mg/kg, QD*21 days | −18% |
| Group 04 | Trametinib, 0.3 mg/kg, QD*21 days | 61% |
| Group 05 | Palbociclib, 75 mg/kg, QD*21 days | 69% |

TABLE 40
Mean tumor volume (mm³) in ZKB182-P2 PDX

| Day | 0 | 3 | 7 | 10 | 14 | 17 |
|---|---|---|---|---|---|---|
| Control | 177.17 ± | 285.80 ± | 453.89 ± | 702.61 ± | 804.40 ± | 1196.94 ± |
| | 42.88 | 57.07 | 93.62 | 212.98 | 217.46 | 362.30 |
| Group 02 | 181.10 ± | 196.11 ± | 183.81 ± | 130.03 ± | 128.19 ± | 158.52 ± |
| | 61.18 | 74.09 | 81.15 | 45.49 | 71.71 | 85.13 |
| Group 03 | 227.72 | 382.48 | 550.41 | 727.16 | 1188.97 | 1357.33 |
| Group 04 | 136.57 | 127.62 | 158.67 | 191.47 | 239.00 | 427.47 |
| Group 05 | 179.71 | 283.89 | 232.39 | 294.17 | 387.64 | 534.72 |
| Day | 21 | 24 | 28 | 31 | 35 | 38 |
| Control | 1377.03 ± | — | — | — | — | — |
| | 301.87 | | | | | |
| Group 02 | 180.76 ± | 235.48 ± | 332.93 ± | 450.23 ± | 596.91 ± | 930.18 ± |
| | 113.82 | 148.28 | 195.48 | 215.99 | 355.47 | 522.63 |
| Group 03 | 1637.98 | — | — | — | — | — |
| Group 04 | 610.82 | 697.40 | 745.33 | 847.45 | — | — |
| Group 05 | 547.91 | 854.88 | | | — | — |

TABLE 41
Mean tumor volume in treated mice versus
control mice (% T/C) in ZKB182-P2 PDX

| Day | 0 | 3 | 7 | 10 | 14 | 17 | 21 |
|---|---|---|---|---|---|---|---|
| Group 02 | 102.22 | 68.62 | 40.50 | 18.51 | 15.94 | 13.24 | 13.13 |
| Group 03 | 128.53 | 133.82 | 121.27 | 103.49 | 147.81 | 113.40 | 118.95 |
| Group 04 | 77.08 | 44.65 | 34.96 | 27.25 | 29.71 | 35.71 | 44.36 |
| Group 05 | 101.43 | 99.33 | 51.20 | 41.87 | 48.19 | 44.67 | 39.79 |

TABLE 42
Tumor Growth Inhibition (TGI %) in ZKB182-P2 PDX

| | Tumor volume at | Tumor volume at | |
|---|---|---|---|
| Group | Day 0 (mm³) | Day 21 (mm³) | TGI % |
| Control | 177.17 ± 42.88 | 1377.03 ± 301.87 | — |
| Group 02 | 181.10 ± 61.18 | 180.76 ± 113.82 | 100% |
| Group 03 | 227.72 | 1637.98 | −18% |
| Group 04 | 136.57 | 610.82 | 61% |
| Group 05 | 179.71 | 547.91 | 69% |

### Example 13 Concentration Dependency of Combination Therapy Cetuximab+Cobimetinib+Palbociclib in CRC Cell Lines with KRAS Mutations

The concentration dependency of the triplet combination cetuximab+cobimetinib+palbociclib was investigated. The same procedures were followed as in Examples 1 and 2. Cell lines used were KRAS^{G12D} CRC cell lines (SNU-407, LS-513, LS-174T, LS180, and CL-40), KRAS^{G12V} CRC cell lines (RCM-1, SK-CO-1, SW403, SW480, SW620), KRAS^{G12A} CRC cell line SW1116, and KRAS^{G13D} CRC cell lines (H747 and HCT-116). PDX model cell pools used were KRAS^{G12C} PDX cell line NYP-041, KRAS^{G12D} PDX cell lines (KY-041 and NYL-233), KRAS^{G12V} PDX cell lines (YL-205 and YP-028), and KRAS^{G13D} PDX cell line NYL-GZ-085. Cell lines were given triplet combinations of an EGFR inhibitor, an MEK 1/2 inhibitor and a CDK 4/6 inhibitor at various MEK 1/2 inhibitor and CDK 4/6 inhibitor concentrations. The MI and growth inhibition rate (GI %) values of the CRC cell lines are shown in Tables 43, 44, and 45.

TABLE 43
Maximum Inhibition Index (MI) and Growth Inhibition Rate
(GI %) during Triplet Combination Therapies for KRAS^{G12C} CRC
(SW1116) and KRAS^{G13D}-CRC (H747 and HCT-116)

| | Concentration | SW1116 | | H747 | | HCT-116 | |
|---|---|---|---|---|---|---|---|
| Samples | (uM) | GI % | MI 4 | GI % | MI 4 | GI % | MI 4 |
| Cetuximab + | 20 + 0.4 + 0.15 | 99.87 | 772.80 | 100.00 | 2135.50 | 98.13 | 53.34 |
| Cobimetinib + | | | | | | | |
| Palbociclib | | | | | | | |
| Cetuximab + | 20 + 0.4 + 0.09 | 100.00 | 1816.55 | 100.00 | 2135.50 | 97.60 | 41.61 |
| Cobimetinib + | | | | | | | |
| ⅗Palbociclib | | | | | | | |
| Cetuximab + | 20 + 0.4 + 0.03 | 100.00 | 1816.55 | 100.00 | 2135.50 | 97.24 | 36.17 |
| Cobimetinib + | | | | | | | |
| ⅕Palbociclib | | | | | | | |
| Cetuximab + | 20 + 0.267 + 0.15 | 100.00 | 1816.55 | 100.00 | 2135.50 | 97.04 | 33.82 |
| ⅔Cobimetinib + | | | | | | | |
| Palbociclib | | | | | | | |
| Cetuximab + | 20 + 0.267 + 0.09 | 100.00 | 1816.55 | 100.00 | 2135.50 | 96.40 | 27.76 |
| ⅔Cobimetinib + | | | | | | | |
| ⅗Palbociclib | | | | | | | |
| Cetuximab + | 20 + 0.267 + 0.03 | 100.00 | 1816.55 | 100.00 | 2135.50 | 92.48 | 13.29 |
| ⅔Cobimetinib + | | | | | | | |
| ⅕Palbociclib | | | | | | | |
| Cetuximab + | 20 + 0.133 + 0.15 | 99.94 | 1787.65 | 100.00 | 2135.50 | 91.89 | 12.33 |
| ⅓Cobimetinib + | | | | | | | |
| Palbociclib | | | | | | | |
| Cetuximab + | 20 + 0.133 + 0.09 | 99.94 | 1792.53 | 100.00 | 2135.50 | 90.65 | 10.70 |
| ⅓Cobimetinib + | | | | | | | |
| ⅗Palbociclib | | | | | | | |
| Cetuximab + | 20 + 0.133 + 0.03 | 99.30 | 143.60 | 100.00 | 2135.50 | 86.82 | 7.59 |
| ⅓Cobimetinib + | | | | | | | |
| ⅕Palbociclib | | | | | | | |
| Osimertinib + | 0.5 + 0.4 + 0.15 | 100.00 | 1816.55 | 100.00 | 2135.50 | 98.84 | 86.41 |
| Cobimetinib + | | | | | | | |
| Palbociclib | | | | | | | |

TABLE 44
Maximum Inhibition Index (MI) and Growth Inhibition Rate (GI %)
during Triplet Combination Therapies for KRAS^{G12D}-CRC cell lines

| | Conc. | SNU-407 | | LS-513 | | LS-174T | | LS180 | | CL-40 | |
|---|---|---|---|---|---|---|---|---|---|---|---|
| Samples | (uM) | GI % | MI 4 | GI % | MI 4 | GI % | MI 4 | GI % | MI 4 | GI % | MI 4 |
| Cetuximab + | 20 + 0.4 + 0.15 | 97.13 | 34.80 | 100.00 | 1635.20 | 94.34 | 17.66 | 98.86 | 87.39 | 99.80 | 493.92 |
| Cobimetinib + | | | | | | | | | | | |
| Palbociclib | | | | | | | | | | | |
| Cetuximab + | 20 + 0.4 + 0.09 | 95.18 | 20.76 | 100.00 | 1635.20 | 92.54 | 13.40 | 97.92 | 48.11 | 99.46 | 185.70 |
| Cobimetinib + | | | | | | | | | | | |
| ⅗Palbociclib | | | | | | | | | | | |
| Cetuximab + | 20 + 0.4 + 0.03 | 91.40 | 11.62 | 100.00 | 1635.20 | 88.02 | 8.35 | 96.43 | 28.03 | 98.59 | 70.84 |
| Cobimetinib + | | | | | | | | | | | |
| ⅕Palbociclib | | | | | | | | | | | |
| Cetuximab + | 20 + 0.267 + 0.15 | 97.13 | 34.81 | 100.00 | 1635.20 | 92.48 | 13.29 | 98.27 | 57.89 | 99.26 | 136.05 |
| ⅔Cobimetinib + | | | | | | | | | | | |
| Palbociclib | | | | | | | | | | | |
| Cetuximab + | 20 + 0.267 + 0.09 | 95.24 | 20.99 | 100.00 | 1635.20 | 89.88 | 9.88 | 97.15 | 35.03 | 98.42 | 63.21 |
| ⅔Cobimetinib + | | | | | | | | | | | |
| ⅗Palbociclib | | | | | | | | | | | |
| Cetuximab + | 20 + 0.267 + 0.03 | 91.63 | 11.94 | 100.00 | 1635.20 | 84.88 | 6.62 | 94.41 | 17.88 | 96.36 | 27.44 |
| ⅔Cobimetinib + | | | | | | | | | | | |
| ⅕Palbociclib | | | | | | | | | | | |
| Cetuximab + | 20 + 0.133 + 0.15 | 95.26 | 21.09 | 100.00 | 1635.20 | 87.22 | 7.83 | 96.12 | 25.76 | 97.03 | 33.65 |
| ⅓Cobimetinib + | | | | | | | | | | | |
| Palbociclib | | | | | | | | | | | |
| Cetuximab + | 20 + 0.133 + 0.09 | 92.77 | 13.83 | 100.00 | 1635.20 | 83.12 | 5.92 | 94.01 | 16.69 | 94.77 | 19.12 |
| ⅓Cobimetinib + | | | | | | | | | | | |
| ⅗Palbociclib | | | | | | | | | | | |
| Cetuximab + | 20 + 0.133 + 0.03 | 87.02 | 7.70 | 100.00 | 1635.20 | 77.71 | 4.49 | 88.68 | 8.83 | 89.05 | 9.14 |
| ⅓Cobimetinib + | | | | | | | | | | | |
| ⅕Palbociclib | | | | | | | | | | | |
| Osimertinib + | 0.5 + 0.4 + 0.15 | 99.39 | 164.76 | 100.00 | 1635.20 | 95.64 | 22.94 | 99.85 | 661.18 | 100.00 | 8465.38 |
| Cobimetinib + | | | | | | | | | | | |
| Palbociclib | | | | | | | | | | | |
| Cetuximab + | 20 + 0.2 + 0.15 | 89.45 | 9.48 | 98.19 | 55.14 | 92.26 | 12.91 | 96.78 | 31.04 | 95.89 | 24.30 |
| Binimetnib + | | | | | | | | | | | |
| Palbociclib | | | | | | | | | | | |
| Cetuximab + | 20 + 0.2 + 0.09 | 85.07 | 6.70 | 96.05 | 25.29 | 89.80 | 9.81 | 94.49 | 18.16 | 93.87 | 16.30 |
| Binimetnib + | | | | | | | | | | | |
| ⅗Palbociclib | | | | | | | | | | | |
| Cetuximab + | 20 + 0.2 + 0.03 | 76.21 | 4.20 | 95.72 | 23.36 | 84.69 | 6.53 | 91.28 | 11.47 | 88.80 | 8.93 |
| Binimetnib + | | | | | | | | | | | |
| ⅕Palbociclib | | | | | | | | | | | |

TABLE 45
Maximum Inhibition Index (MI) and Growth Inhibition Rate (GI %)
during Triplet Combination Therapies for KRAS^{G12V}-CRC cell lines

| | Conc. | RCM-1 | | SK-CO-1 | | SW403 | | SW480 | | SW620 | |
|---|---|---|---|---|---|---|---|---|---|---|---|
| Samples | (uM) | GI % | MI 4 | GI % | MI 4 | GI % | MI 4 | GI % | MI 4 | GI % | MI 4 |
| Cetuximab + | 20 + 0.4 + 0.15 | 100.00 | 2638.53 | 99.94 | 1576.87 | 100.00 | 2446.64 | 110.42 | 99.09 | 100.00 | 6353.48 |
| Cobimetinib + | | | | | | | | | | | |
| Palbociclib | | | | | | | | | | | |
| Cetuximab + | 20 + 0.4 + 0.09 | 99.95 | 2035.38 | 99.99 | 9539.42 | 100.00 | 2446.64 | 46.80 | 97.86 | 100.00 | 6353.48 |
| Cobimetinib + | | | | | | | | | | | |
| ⅗Palbociclib | | | | | | | | | | | |
| Cetuximab + | 20 + 0.4 + 0.03 | 99.86 | 720.50 | 99.93 | 1413.26 | 100.00 | 2446.64 | 16.96 | 94.10 | 99.87 | 758.68 |
| Cobimetinib + | | | | | | | | | | | |
| ⅕Palbociclib | | | | | | | | | | | |
| Cetuximab + | 20 + 0.267 + 0.15 | 100.00 | 2638.53 | 99.96 | 2808.88 | 100.00 | 2446.64 | 41.53 | 97.59 | 99.98 | 6235.02 |
| ⅔Cobimetinib + | | | | | | | | | | | |
| Palbociclib | | | | | | | | | | | |
| Cetuximab + | 20 + 0.267 + 0.09 | 99.80 | 495.88 | 99.96 | 2359.89 | 100.00 | 2446.64 | 21.43 | 95.33 | 100.00 | 6353.48 |
| ⅔Cobimetinib + | | | | | | | | | | | |
| ⅗Palbociclib | | | | | | | | | | | |
| Cetuximab + | 20 + 0.267 + 0.03 | 99.58 | 240.18 | 99.85 | 674.28 | 100.00 | 2446.64 | 9.90 | 89.90 | 99.80 | 487.90 |
| ⅔Cobimetinib + | | | | | | | | | | | |
| ⅕Palbociclib | | | | | | | | | | | |
| Cetuximab + | 20 + 0.133 + 0.15 | 98.91 | 92.09 | 99.93 | 1414.57 | 100.00 | 2446.64 | 15.39 | 93.50 | 100.00 | 6353.48 |
| ⅓Cobimetinib + | | | | | | | | | | | |
| Palbociclib | | | | | | | | | | | |
| Cetuximab + | 20 + 0.133 + 0.09 | 98.43 | 63.86 | 99.85 | 673.92 | 100.00 | 2446.64 | 10.55 | 90.53 | 99.96 | 2742.91 |
| ⅓Cobimetinib + | | | | | | | | | | | |
| ⅗Palbociclib | | | | | | | | | | | |
| Cetuximab + | 20 + 0.133 + 0.03 | 96.45 | 28.18 | 97.62 | 42.07 | 100.00 | 2446.64 | 7.14 | 86.00 | 99.69 | 323.66 |
| ⅓Cobimetinib + | | | | | | | | | | | |
| ⅕Palbociclib | | | | | | | | | | | |
| Osimertinib + | 0.5 + 0.4 + 0.15 | 100.00 | 2638.53 | 99.99 | 7054.58 | 100.00 | 2446.64 | 155.87 | 99.36 | 100.00 | 6353.48 |
| Cobimetinib + | | | | | | | | | | | |
| Palbociclib | | | | | | | | | | | |
| Cetuximab + | 20 + 0.2 + 0.15 | 94.38 | 17.78 | 99.79 | 470.83 | 100.00 | 2446.64 | 7.03 | 85.77 | 99.63 | 268.18 |
| Binimetnib + | | | | | | | | | | | |
| Palbociclib | | | | | | | | | | | |
| Cetuximab + | 20 + 0.2 + 0.09 | 90.79 | 10.86 | 99.43 | 176.64 | 100.00 | 2446.64 | 5.22 | 80.84 | 98.46 | 64.82 |
| Binimetnib + | | | | | | | | | | | |
| ⅗Palbociclib | | | | | | | | | | | |
| Cetuximab + | 20 + 0.2 + 0.03 | 87.53 | 8.02 | 90.93 | 11.02 | 100.00 | 2446.64 | 3.61 | 72.33 | 93.25 | 14.81 |
| Binimetnib + | | | | | | | | | | | |
| ⅕Palbociclib | | | | | | | | | | | |

As shown in Tables 43-45, combined inhibition of EGFR, MEK1/2 and CDK4/6 suppressed tumor cell growth across wide range of KRAS mutant CRC cell lines. Reduction of a single drug had less impact on the tumor inhibitory effect compared to reduction of two drugs. Dose reduction of both cobimetinib and palbociclib decreased the Growth Inhibition Rate (GI %) in all CRC cell lines by less than 13% at 1/3 cobimetinib and 1/5 palbociclib concentrations. Remarkably, growth inhibitions of KRAS^{G12C} CRC (SW1116), KRAS^{G13D}-CRC (H747), KRAS^{G12D}-CRC (LS-513), and KRAS^{G12V}-CRC (RCM-1, SK-CO-1, SW403) were not impacted at cobimetinib and palbociclib concentrations as low as 1/3 and 1/5 of the full dose, respectively. These results demonstrated that the amount of drugs used in the combination therapy can be significantly reduced without compromising clinical efficacy. Reduction of the dose may reduce drug-related toxicity and side effects, and further help improve patient compliance.

### Example 14. Concentration Dependency Combination Therapy Osimertinib+Cobimetinib+Palbociclib in CRC Cell Lines with KRAS Mutations

The concentration dependency of the triplet combination osimertinib+cobimetinib+palbociclib was investigated. The same procedures were followed as in Example 13. Cell lines used were KRAS^{G12D} CRC cell lines (SNU-407, LS-513, LS-174T, LS180, and CL-40), KRAS^{G12V} CRC cell lines (RCM-1, SK-CO-1, SW403, SW480, SW620), KRAS^{G12A} CRC cell line SW1116, and KRAS^{G13D} CRC cell line (H747). Cell lines were given triplet combinations of osimertinib+cobimetinib+palbociclib at various cobimetinib and palbociclib concentrations. The MI and growth inhibition rate (GI %) values of the CRC cell lines are shown in Tables 46, 47, and 48.

TABLE 46
Maximum Inhibition Index (MI) and Growth Inhibition Rate (GI %)
during Triplet Combination Therapy Osimertinib + Cobimetinib +
Palbociclib for KRAS^{G12C} CRC (SW1116) and KRAS^{G13D}-CRC (H747)

| | Concentration | SW1116 | | H747 | |
|---|---|---|---|---|---|
| Samples | (UM) | GI % | MI 4 | GI % | MI 4 |
| Osimertinib + Cobimetinib + | 0.5 + 0.4 + 0.15 | 100.00 | 1551.44 | 100.00 | 1943.74 |
| Palbociclib | | | | | |
| Osimertinib + Cobimetinib + | 0.5 + 0.4 + 0.12 | 100.00 | 1551.44 | 100.00 | 1943.74 |
| Palbociclib | | | | | |
| Osimertinib + Cobimetinib + | 0.5 + 0.4 + 0.09 | 100.00 | 1551.44 | 100.00 | 1943.74 |
| Palbociclib | | | | | |
| Osimertinib + Cobimetinib + | 0.5 + 0.4 + 0.06 | 100.00 | 1551.44 | 100.00 | 1943.74 |
| Palbociclib | | | | | |
| Osimertinib + Cobimetinib + | 0.5 + 0.4 + 0.03 | 100.00 | 1551.44 | 100.00 | 1943.74 |
| Palbociclib | | | | | |
| Osimertinib + Cobimetinib + | 0.5 + 0.27 + 0.15 | 100.00 | 1551.44 | 100.00 | 1943.74 |
| Palbociclib | | | | | |
| Osimertinib + Cobimetinib + | 0.5 + 0.27 + 0.12 | 100.00 | 1551.44 | 100.00 | 1943.74 |
| Palbociclib | | | | | |
| Osimertinib + Cobimetinib + | 0.5 + 0.27 + 0.09 | 100.00 | 1551.44 | 100.00 | 1943.74 |
| Palbociclib | | | | | |
| Osimertinib + Cobimetinib + | 0.5 + 0.27 + 0.06 | 100.00 | 1551.44 | 100.00 | 1943.74 |
| Palbociclib | | | | | |
| Osimertinib + Cobimetinib + | 0.5 + 0.27 + 0.03 | 100.00 | 1551.44 | 100.00 | 1943.74 |
| Palbociclib | | | | | |
| Osimertinib + Cobimetinib + | 0.5 + 0.13 + 0.15 | 100.00 | 1551.44 | 100.00 | 1943.74 |
| Palbociclib | | | | | |
| Osimertinib + Cobimetinib + | 0.5 + 0.13 + 0.12 | 100.00 | 1551.44 | 100.00 | 1943.74 |
| Palbociclib | | | | | |
| Osimertinib + Cobimetinib + | 0.5 + 0.13 + 0.09 | 100.00 | 1551.44 | 100.00 | 1943.74 |
| Palbociclib | | | | | |
| Osimertinib + Cobimetinib + | 0.5 + 0.13 + 0.06 | 100.00 | 1551.44 | 100.00 | 1943.74 |
| Palbociclib | | | | | |
| Osimertinib + Cobimetinib + | 0.5 + 0.13 + 0.03 | 100.00 | 1551.60 | 100.00 | 1943.74 |
| Palbociclib | | | | | |

TABLE 47
Maximum Inhibition Index (MI) and Growth Inhibition Rate (GI %) during Triplet Combination
Therapies Osimertinib + Cobimetinib + Palbociclib for KRAS^{G12D}-CRC cell lines

| | Conc. | SNU-407 | | LS-513 | | LS-174T | | LS180 | | CL-40 | |
|---|---|---|---|---|---|---|---|---|---|---|---|
| Samples | (uM) | MI 4 | GI % | MI 4 | GI % | MI 4 | GI % | MI 4 | GI % | MI 4 | GI % |
| Osimertinib + | 0.5 + 0.4 + 0.15 | 189.61 | 99.47 | 1505.61 | 100.00 | 22.59 | 95.57 | 535.00 | 99.81 | 8929.11 | 100.00 |
| Cobimetinib + | | | | | | | | | | | |
| Palbociclib | | | | | | | | | | | |
| Osimertinib + | 0.5 + 0.4 + 0.12 | 104.18 | 99.04 | 1505.61 | 100.00 | 16.20 | 93.83 | 333.23 | 99.70 | 8929.11 | 100.00 |
| Cobimetinib + | | | | | | | | | | | |
| Palbociclib | | | | | | | | | | | |
| Osimertinib + | 0.5 + 0.4 + 0.09 | 93.65 | 98.93 | 1505.61 | 100.00 | 14.63 | 93.16 | 196.01 | 99.49 | 8929.11 | 100.00 |
| Cobimetinib + | | | | | | | | | | | |
| Palbociclib | | | | | | | | | | | |
| Osimertinib + | 0.5 + 0.4 + 0.06 | 64.66 | 98.45 | 1505.61 | 100.00 | 11.99 | 91.66 | 79.14 | 98.74 | 3856.57 | 99.97 |
| Cobimetinib + | | | | | | | | | | | |
| Palbociclib | | | | | | | | | | | |
| Osimertinib + | 0.5 + 0.4 + 0.03 | 26.52 | 96.23 | 1505.61 | 100.00 | 8.68 | 88.48 | 37.87 | 97.36 | 1779.00 | 99.94 |
| Cobimetinib + | | | | | | | | | | | |
| Palbociclib | | | | | | | | | | | |
| Osimertinib + | 0.5 + 0.27 + 0.15 | 117.32 | 99.15 | 1505.61 | 100.00 | 13.46 | 92.57 | 128.83 | 99.22 | 3894.81 | 99.97 |
| Cobimetinib + | | | | | | | | | | | |
| Palbociclib | | | | | | | | | | | |
| Osimertinib + | 0.5 + 0.27 + 0.12 | 89.91 | 98.89 | 1505.61 | 100.00 | 12.52 | 92.02 | 124.26 | 99.20 | 2649.90 | 99.96 |
| Cobimetinib + | | | | | | | | | | | |
| Palbociclib | | | | | | | | | | | |
| Osimertinib + | 0.5 + 0.27 + 0.09 | 66.55 | 98.50 | 1505.61 | 100.00 | 11.01 | 90.91 | 90.52 | 98.90 | 2682.84 | 99.96 |
| Cobimetinib + | | | | | | | | | | | |
| Palbociclib | | | | | | | | | | | |
| Osimertinib + | 0.5 + 0.27 + 0.06 | 42.01 | 97.62 | 1505.61 | 100.00 | 9.15 | 89.07 | 47.31 | 97.89 | 1290.15 | 99.92 |
| Cobimetinib + | | | | | | | | | | | |
| Palbociclib | | | | | | | | | | | |
| Osimertinib + | 0.5 + 0.27 + 0.03 | 23.77 | 95.79 | 1505.61 | 100.00 | 6.96 | 85.62 | 20.24 | 95.06 | 408.34 | 99.76 |
| Cobimetinib + | | | | | | | | | | | |
| Palbociclib | | | | | | | | | | | |
| Osimertinib + | 0.5 + 0.13 + 0.15 | 59.12 | 98.31 | 1505.61 | 100.00 | 8.41 | 88.10 | 39.03 | 97.44 | 1161.39 | 99.91 |
| Cobimetinib + | | | | | | | | | | | |
| Palbociclib | | | | | | | | | | | |
| Osimertinib + | 0.5 + 0.13 + 0.12 | 52.87 | 98.11 | 1505.61 | 100.00 | 7.63 | 86.90 | 38.04 | 97.37 | 430.48 | 99.77 |
| Cobimetinib + | | | | | | | | | | | |
| Palbociclib | | | | | | | | | | | |
| Osimertinib + | 0.5 + 0.13 + 0.09 | 40.61 | 97.54 | 1505.61 | 100.00 | 6.23 | 83.96 | 35.08 | 97.15 | 326.88 | 99.69 |
| Cobimetinib + | | | | | | | | | | | |
| Palbociclib | | | | | | | | | | | |
| Osimertinib + | 0.5 + 0.13 + 0.06 | 28.32 | 96.47 | 1505.61 | 100.00 | 5.75 | 82.61 | 26.70 | 96.25 | 166.69 | 99.40 |
| Cobimetinib + | | | | | | | | | | | |
| Palbociclib | | | | | | | | | | | |
| Osimertinib + | 0.5 + 0.13 + 0.03 | 15.41 | 93.51 | 1505.61 | 100.00 | 4.38 | 77.15 | 16.51 | 93.94 | 65.41 | 98.47 |
| Cobimetinib + | | | | | | | | | | | |
| Palbociclib | | | | | | | | | | | |

TABLE 48
Maximum Inhibition Index (MI) and Growth Inhibition Rate (GI %) during Triplet Combination
Therapies Osimertinib + Cobimetinib + Palbociclib for KRAS^{G12V}-CRC cell lines

| | Conc. | RCM-1 | | SK-CO-1 | | SW403 | | SW480 | | SW620 | |
|---|---|---|---|---|---|---|---|---|---|---|---|
| Samples | (uM) | MI 4 | GI % | MI 4 | GI % | MI 4 | GI % | MI 4 | GI % | MI 4 | GI % |
| Osimertinib + | 0.5 + 0.4 + 0.15 | 2301.38 | 100.00 | 2639.99 | 99.96 | 2642.53 | 100.00 | 1014.46 | 99.90 | 2504.77 | 99.96 |
| Cobimetinib + | | | | | | | | | | | |
| Palbociclib | | | | | | | | | | | |
| Osimertinib + | 0.5 + 0.4 + 0.12 | 2301.38 | 100.00 | 3964.09 | 99.97 | 2642.53 | 100.00 | 233.91 | 99.57 | 5793.54 | 100.00 |
| Cobimetinib + | | | | | | | | | | | |
| Palbociclib | | | | | | | | | | | |
| Osimertinib + | 0.5 + 0.4 + 0.09 | 2301.38 | 100.00 | 3167.65 | 99.97 | 2642.53 | 100.00 | 166.18 | 99.40 | 5733.91 | 99.98 |
| Cobimetinib + | | | | | | | | | | | |
| Palbociclib | | | | | | | | | | | |
| Osimertinib + | 0.5 + 0.4 + 0.06 | 2301.38 | 100.00 | 5280.69 | 100.00 | 2642.53 | 100.00 | 51.22 | 98.05 | 5793.54 | 100.00 |
| Cobimetinib + | | | | | | | | | | | |
| Palbociclib | | | | | | | | | | | |
| Osimertinib + | 0.5 + 0.4 + 0.03 | 2301.38 | 100.00 | 5280.69 | 100.00 | 2642.53 | 100.00 | 19.17 | 94.78 | 5793.54 | 100.00 |
| Cobimetinib + | | | | | | | | | | | |
| Palbociclib | | | | | | | | | | | |
| Osimertinib + | 0.5 + 0.27 + 0.15 | 2301.38 | 100.00 | 1980.90 | 99.95 | 2642.53 | 100.00 | 269.35 | 99.63 | 5793.54 | 100.00 |
| Cobimetinib + | | | | | | | | | | | |
| Palbociclib | | | | | | | | | | | |
| Osimertinib + | 0.5 + 0.27 + 0.12 | 2287.35 | 99.96 | 5246.07 | 99.98 | 2642.53 | 100.00 | 169.93 | 99.41 | 5793.54 | 100.00 |
| Cobimetinib + | | | | | | | | | | | |
| Palbociclib | | | | | | | | | | | |
| Osimertinib + | 0.5 + 0.27 + 0.09 | 2301.38 | 100.00 | 5277.17 | 99.98 | 2642.53 | 100.00 | 71.45 | 98.60 | 5793.54 | 100.00 |
| Cobimetinib + | | | | | | | | | | | |
| Palbociclib | | | | | | | | | | | |
| Osimertinib + | 0.5 + 0.27 + 0.06 | 688.56 | 99.85 | 5280.69 | 100.00 | 2642.53 | 100.00 | 25.67 | 96.11 | 5793.54 | 100.00 |
| Cobimetinib + | | | | | | | | | | | |
| Palbociclib | | | | | | | | | | | |
| Osimertinib + | 0.5 + 0.27 + 0.03 | 575.47 | 99.83 | 5263.85 | 99.98 | 2642.53 | 100.00 | 9.90 | 89.90 | 5793.54 | 100.00 |
| Cobimetinib + | | | | | | | | | | | |
| Palbociclib | | | | | | | | | | | |
| Osimertinib + | 0.5 + 0.13 + 0.15 | 345.62 | 99.71 | 1317.85 | 99.92 | 2642.53 | 100.00 | 44.80 | 97.77 | 5793.54 | 100.00 |
| Cobimetinib + | | | | | | | | | | | |
| Palbociclib | | | | | | | | | | | |
| Osimertinib + | 0.5 + 0.13 + 0.12 | 197.38 | 99.49 | 2241.16 | 99.96 | 2642.53 | 100.00 | 25.22 | 96.03 | 5793.54 | 100.00 |
| Cobimetinib + | | | | | | | | | | | |
| Palbociclib | | | | | | | | | | | |
| Osimertinib + | 0.5 + 0.13 + 0.09 | 191.61 | 99.48 | 2631.14 | 99.96 | 2642.53 | 100.00 | 15.63 | 93.60 | 5793.54 | 100.00 |
| Cobimetinib + | | | | | | | | | | | |
| Palbociclib | | | | | | | | | | | |
| Osimertinib + | 0.5 + 0.13 + 0.06 | 130.43 | 99.23 | 3199.13 | 99.97 | 2642.53 | 100.00 | 9.43 | 89.40 | 5793.54 | 100.00 |
| Cobimetinib + | | | | | | | | | | | |
| Palbociclib | | | | | | | | | | | |
| Osimertinib + | 0.5 + 0.13 + 0.03 | 75.14 | 98.67 | 1746.20 | 99.94 | 2642.53 | 100.00 | 7.22 | 86.15 | 1590.18 | 99.94 |
| Cobimetinib + | | | | | | | | | | | |
| Palbociclib | | | | | | | | | | | |

As Tables 46-48 evidently show, osimertinib+cobimetinib+palbociclib demonstrated remarkable tumor cell growth suppression in all KRAS mutant CRC cell lines tested of nearly 100%, even at cobimetinib and palbociclib concentrations as low as 1/3 and 1/5 of the full dose, respectively. These results once again confirmed that the amount of drugs used in the combination therapy can be significantly reduced without compromising clinical efficacy. Reduction of the dose may reduce drug-related toxicity and side effects, and further help improve patient compliance.

### Example 15. Tumor Volume Reduction in Mice During Combination Therapy in a PDX Xenograft Model with Patient HCT-116 (G13D)

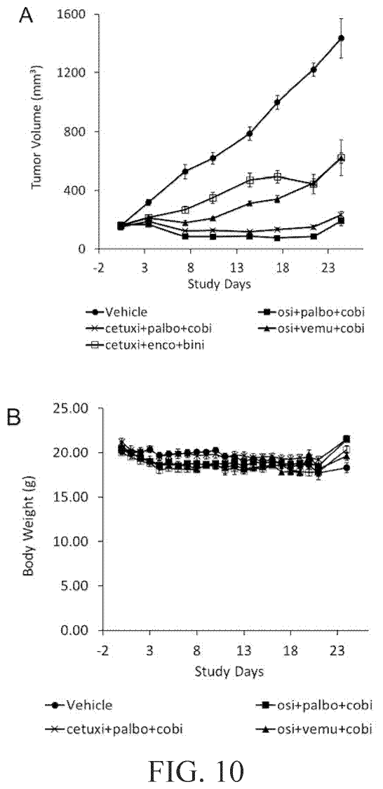

The same procedures were followed as in Example 3 with KRAS^{G13D} CRC patient HCT-116 using various combinations as indicated. The drugs used are shown in Table 49. The mean tumor volumes are shown in FIG. 10A. The body weights of each group of mice are shown in FIG. 10B. At Day 21, the triplet combinations with EGFR+MEK1/2+CDK4/6 inhibitors (osimertinib+cobimetinib+palbociclib and cetuximab+cobimetinib+palbociclib) both showed better tumor volume reduction activities compared to when BRAF inhibitors were used.

TABLE 49
Drugs used in the study

| Name | Abbreviation | Target | Dose, route and schedule |
|---|---|---|---|
| cetuximab | cetux1 | EGFR | 1 mg/kg, ip, QW × 3 weeks |
| osimertinib | osi | EGFR | 10 mg/kg, po, QD × 21 days |
| cobimetinib | cobi | MEK1/2 | 5 mg/kg, po, QD × 21 days |
| binimetinib | bini | MEK1/2 | 30 mg/kg, po, BID × 21 days |
| palbociclib | palbo | CDK4/6 | 20 mg/kg, po, QD × 21 days |
| vemurafenib | vemu | BRAF | 100 mg/kg, po, QD × 21 days |
| encorafenib | enco | BRAF | 40 mg/kg, po, QD × 21 days |

### Example 16. Apoptosis Assay with KRAS Mutant Colorectal Cancer Cells Using Cetuximab+Cobimetinib+Palbociclib

Cellular apoptosis assays were performed with Alexa Flour™ 488 Annexin V/Dead cell apoptosis Kit (ThermoFisher, USA) and GreenNuc™ live cell caspase 3 activity assay kit (Beyotime Biotechnology, China). CRC cell lines H747 (G13D), HCT-116 (G13D), SW480 (G12V), LS180 (G12D) were used. The experimental design is shown in Table 50. CCP stands for cetuximab+cobimetinib+palbociclib. Briefly, cells cultured in 6-well tissue culture plates were treated with the designated drugs or DMSO, respectively. After 72-hours incubation, the cells were collected and assayed for apoptosis according to manufacturers' instructions. For annexin V assay, the cell suspensions were incubated with Alexa Flour™ 488 labeled annexin V and Propidium Iodide (PI). For the caspase 3 activity assay, a caspase 3 substrate was added to generate fluorescent product. The cells were then analyzed on a BD LSRFortessa cell analyzer. Flow cytometry data were analyzed with Flowjo software.

TABLE 50
Apoptosis assay with KRAS mutant colorectal cells.

| | | % Apoptotic cells | | % Apototic cells | |
|---|---|---|---|---|---|
| | KRAS | (annexin V) | | (caspase 3) | |
| Cell lines | Mutation | CCP | Vehicle | CCP | Vehicle |
| NCI-H747 | G13D | 26 | 7.89 | 38 | 6.08 |
| HCT-116 | G13D | 6.66 | 4.84 | 4.44 | 1.75 |
| SW480 | G12V | 7.37 | 0.86 | n/a | n/a |
| LS180 | G12D | 10.5 | 8.32 | n/a | n/a |

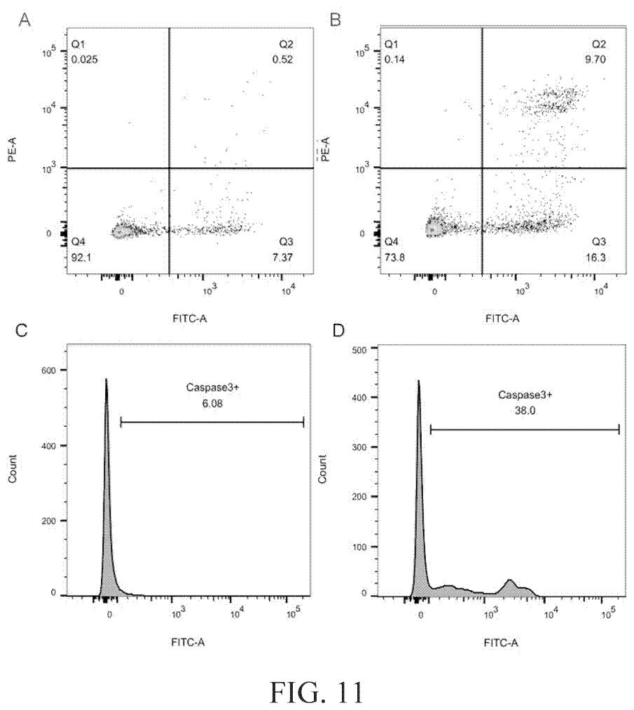

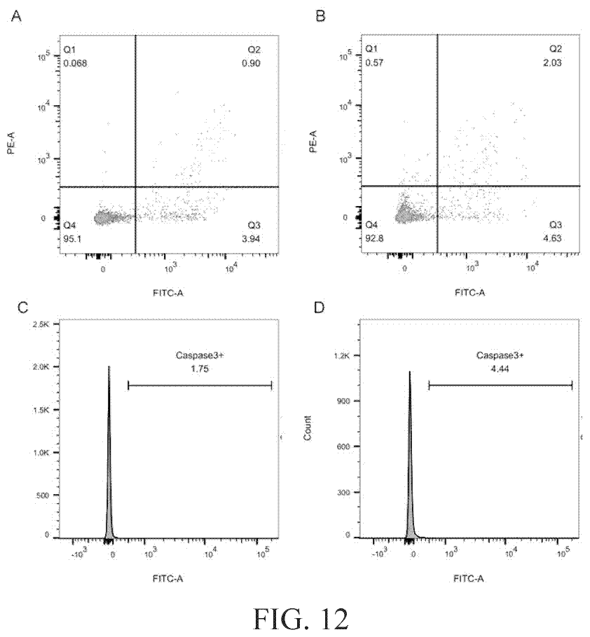

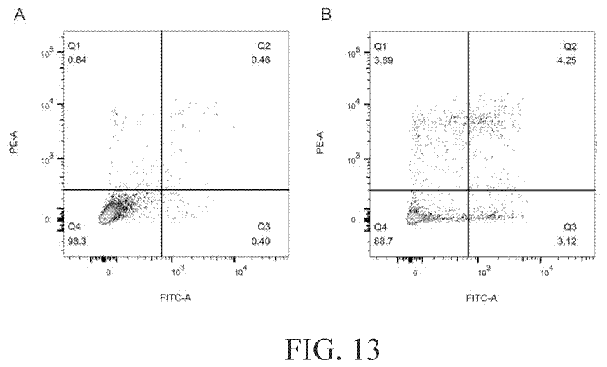

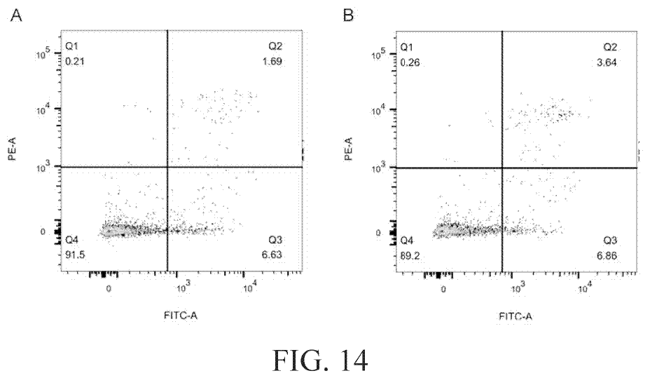

The results are shown in FIGS. 11-14. The triplet combination cetuximab+cobimetinib+palbociclib induced apoptosis in KRAS mutant CRC cell lines NCI-H747 (G13D) cells, which showed substantial apoptosis upon cetuximab+cobimetinib+palbociclib treatment.

### Example 17. Cellular Senescence Assay with KRAS Mutant Colorectal Cancer Cells Using Cetuximab+Cobimetinib+Palbociclib

Senescence is a state when cells enter a stable growth arrest. Unlike a quiescent cell, a senescent cell will not re-enter the cell cycle in response to any known physiological stimuli. A biomarker of senescence is the increased level of β-galactosidase activity at pH 6.0.

Cellular senescence assay was performed with Senescence β-Galactosidase Cell Staining Kit (Cell Signaling, USA) according to the manufacturer's instructions. Briefly, cells cultured in 6-well tissue culture plates were treated with the designated drugs or DMSO. The cells were continually incubated for additional 4-8 days before being fixed for further testing. For senescence assay, b-Galactosidase Staining Solution was added to the fixed cells and the plates were incubated at 37° C. to allow color to develop. Microscopic images were then taken and the cells in senescence were indicated by the development of blue color (shown as black spots in the figures). The experimental design is the same as in Example 15. In separate examples, treatment of single drugs etoposide or doxorubicin did not induce cell senescence.

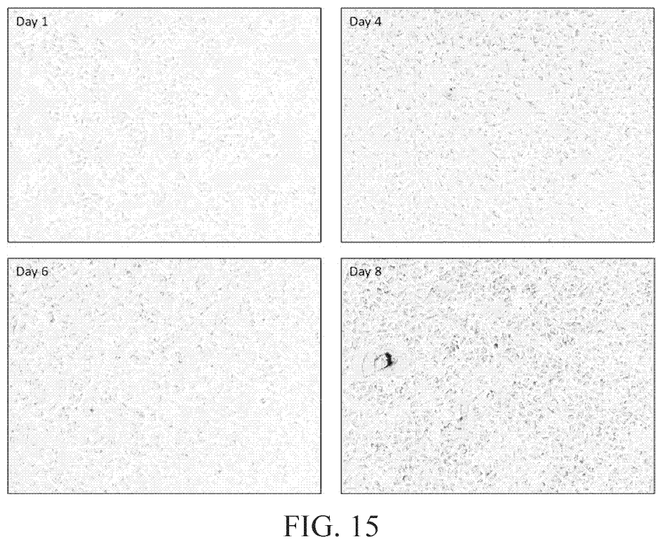

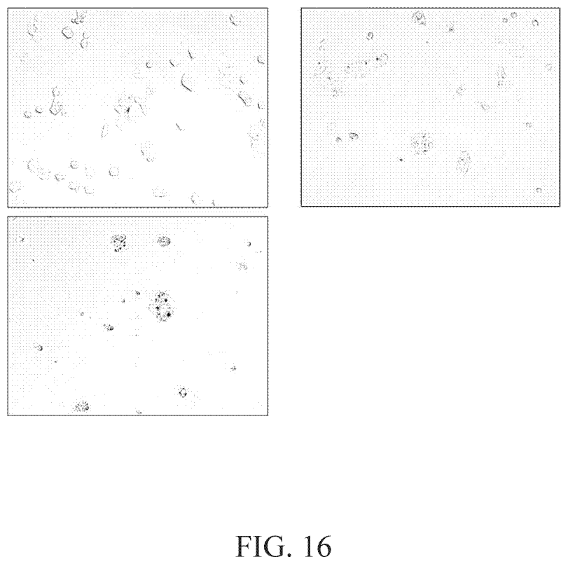

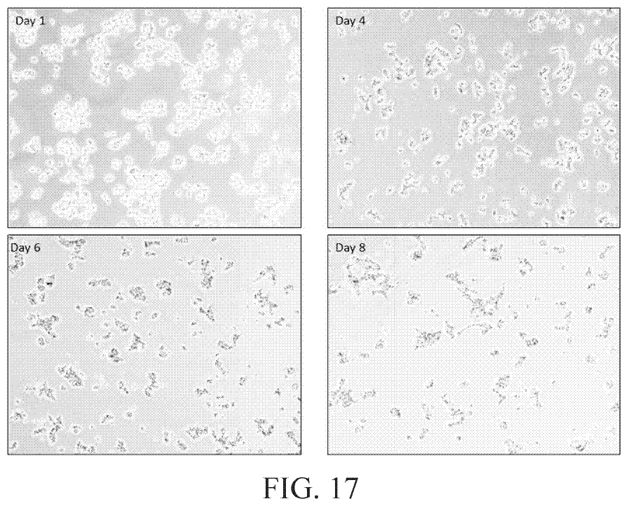

The results are shown in FIGS. 15-17. In HCT-116 (G13D), SW480 (G12V) and LS180 (G12D), majority of the cells went into senescence after treatment of the triplet combination cetuximab+cobimetinib+palbociclib induced. In summary, in all four KRAS mutant CRC cell lines H747 (G13D), HCT-116 (G13D), SW480 (G12V), LS180 (G12D), cetuximab+cobimetinib+palbociclib induced either apoptosis or senescence. The triplet combination cetuximab+cobimetinib+palbociclib demonstrated potent anti-tumor activities by not only inhibiting growth of tumor cells, but also potently inducing cell apoptosis in KRAS mutant CRC cell lines.

What is claimed is:

1. A method of treating or delaying progression of colorectal cancer in a subject comprising administering to the subject an affective amount of
(a) an epidermal growth factor receptor (EGFR) inhibitor;
(b) a mitogen-activated protein kinase (MEK) 1/2 inhibitor; and
(c) a cyclin dependent kinase (CDK) 4/6 inhibitor;
wherein the subject has colorectal cancer that has a KRAS mutation or is at risk of developing colorectal cancer that has a KRAS mutation.

2. The method of claim 1, wherein a KRAS inhibitor is not administered to the subject.

3. The method of claim 1 or claim 2, wherein the EGFR inhibitor is osimertinib or a salt thereof, lapatinib or a salt thereof, or cetuximab, wherein the MEK 1/2 inhibitor is cobimetinib or a salt thereof, trametinib or a salt thereof, binimetinib or a salt thereof, or TAK-733 or a salt thereof, and wherein the CDK 4/6 inhibitor is palbociclib or a salt thereof, or abemaciclib or a salt thereof.

4. The method of any one of claims 1-3, wherein the method comprises administering an effective amount of osimertinib or a salt thereof, cobimetinib or a salt thereof, and palbociclib or a salt thereof.

5. The method of any one of claims 1-3, wherein the method comprises administering to the subject an effective amount of osimertinib or a salt thereof, TAK-733 or a salt thereof, and palbociclib or a salt thereof.

6. The method of any one of claims 1-3, wherein the method comprises administering to the subject an effective amount of osimertinib or a salt thereof, trametinib or a salt thereof, and palbociclib or a salt thereof.

7. The method of any one of claims 1-3, wherein the method comprises administering to the subject an effective amount of cetuximab, cobimetinib or a salt thereof, and palbociclib or a salt thereof.

8. The method of any one of claims 1-3, wherein the method comprises administering to the subject an effective amount of cetuximab, TAK-733 or a salt thereof, and palbociclib or a salt thereof.

9. The method of any one of claims 1-3, wherein the method comprises administering to the subject an effective amount of cetuximab, trametinib or a salt thereof, and palbociclib or a salt thereof.

10. The method of any one of claims 1-3, wherein the method comprises administering to the subject an effective amount of cetuximab, cobimetinib or a salt thereof, and abemaciclib or a salt thereof.

11. The method of any one of claims 1-3, wherein the method comprises administering to the subject an effective amount of cetuximab, binimetinib or a salt thereof, and palbociclib or a salt thereof.

12. The method of any one of claims 1-3, wherein the method comprises administering to the subject an effective amount of osimertinib, binimetinib or a salt thereof, and palbociclib or a salt thereof

13. The method of claim 4, wherein osimertinib or a salt thereof, cobimetinib or a salt thereof, and palbociclib or a salt thereof are administered in one composition.

14. The method of claim 4, wherein osimertinib or a salt thereof, cobimetinib or a salt thereof, and palbociclib or a salt thereof are administered simultaneously to the subject.

15. The method of claim 4, wherein osimertinib or a salt thereof, cobimetinib or a salt thereof, and palbociclib or a salt thereof are administered intermittently to the subject.

16. The method of claim 7, wherein cetuximab, cobimetinib or a salt thereof, and palbociclib or a salt thereof are administered in two or more compositions.

17. The method of claim 8, wherein cetuximab, TAK-733 or a salt thereof, and palbociclib or a salt thereof are administered in two or more compositions.

18. The method of any one of claims 1-3, wherein the method comprises administering to the subject of lapatinib or a salt thereof, a trametinib or a salt thereof, and palbociclib or a salt thereof.

19. The method of any one of claims 1-18, wherein the EGFR inhibitor, MEK 1/2 inhibitor, and the CDK 4/6 inhibitor are administered in one composition or two or more compositions, administered simultaneously to the subject, administered separately to the subject or administered intermittently to the subject.

20. The method of any one of claims 1-19, wherein the cancer has a KRAS G12 mutation or a KRAS G13 mutation.

21. The method of claim 20, wherein the KRAS G12 mutation is G12A, G12V, G12C, or G12D mutation.

22. The method of claim 20, wherein the KRAS G13 mutation is G13D mutation.

23. The method of claim 20, wherein the KRAS has a KRAS G12V or G13D mutation.

24. The method of any one of claims 1-23, wherein the cancer is a malignant epithelial tumor or carcinoma.

25. The method of any one of claims 1-24, wherein the method reduces cancer cell growth and/or increase cancer cell-killing by about 20-99% more than administration of (a) the EGFR inhibitor, (b) the MEK 1/2 inhibitor or (c) the CDK 4/6 inhibitor alone.

26. The method of any one of claims 1-25, wherein the method reduces mean tumor volume by about 20-95%.

27. The method of any one of claims 3-6 and 12-15, wherein osimertinib or a salt thereof is administered to the subject in a daily dose of about 40-80 mg.

28. The method of any one of claim 3, 4, 7, 10, and 13-16, wherein cobimetinib or a salt thereof is administered to the subject in a daily dose of about 20-60 mg.

29. The method of any one of claims 3-9 and 11-18, wherein palbociclib or a salt thereof is administered to the subject in a daily dose of about 75-125 mg.

30. The method of any one of claims 7-11 and 16-17, wherein cetuximab is administered to the subject in a weekly dose of about 400 mg/m² infused over 120 minutes with a maximum infusion rate of 10 mg/min, followed by weekly dose of 250 mg/m² infused over 60 minutes with a maximum infusion rate of 10 mg/min.

31. The method of any one of claims 1-30, wherein the subject is a human.

32. The method of any one of claims 1-31, wherein the method reduces the tumor volume by at least about 85%.

33. A composition for use in treating or delaying progression of colorectal cancer in a subject comprising an affective amount of;
(a) an epidermal growth factor receptor (EGFR) inhibitor;
(b) a mitogen-activated protein kinase (MEK) 1/2 inhibitor; and
(c) a cyclin dependent kinase (CDK) 4/6 inhibitor;
wherein the subject has colorectal cancer that has a KRAS mutation or is at risk of developing colorectal cancer that has a KRAS mutation.

34. The composition for use of claim 33, wherein the composition does not comprise a KRAS inhibitor.

35. The composition for use of claim 33 or 34, wherein the EGFR inhibitor is osimertinib or a salt thereof, lapatinib or a salt there of, or cetuximab, wherein the MEK 1/2 inhibitor is cobimetinib or a salt thereof, trametinib or a salt thereof, binimetinib or a salt thereof, or TAK-733 or a salt thereof, and wherein the CDK 4/6 inhibitor is palbociclib, or a salt thereof or abemaciclib or a salt thereof.

36. The composition for use of any one of claims 33-35, wherein the composition comprises an affective amount of osimertinib or a salt thereof, cobimetinib or a salt thereof, and palbociclib or a salt thereof.

37. The composition for use of any one of claims 33-35, wherein the composition comprises an effective amount of osimertinib or a salt thereof, TAK-733 or a salt thereof, and palbociclib or a salt thereof.

38. The composition for use of any one of claims 33-35, wherein the composition comprises an effective amount of osimertinib or a salt thereof, trametinib or a salt thereof, and palbociclib or a salt thereof.

39. The composition for use of any one of claims 33-35, wherein the composition comprises an effective amount of cetuximab, cobimetinib or a salt thereof, and palbociclib or a salt thereof.

40. The composition for use of any one of claims 33-35, wherein the composition comprises an effective amount of cetuximab, TAK-733 or a salt thereof, and palbociclib or a salt thereof.

41. The composition for use of any one of claims 33-35, wherein the composition comprises an effective amount of cetuximab, trametinib or a salt thereof, and palbociclib or a salt thereof.

42. The composition for use of any one of claims 33-35, wherein the composition comprises an effective amount of lapatinib or a salt thereof, trametinib or a salt thereof, and palbociclib or a salt thereof.

43. The composition for use of any one of claims 33-35, wherein the composition comprises an effective amount of cetuximab, cobimetinib or a salt thereof, and abemaciclib or a salt thereof.

44. The composition for use of any one of claims 33-35, wherein the composition comprises an effective amount of cetuximab, binimetinib or a salt thereof, and palbociclib or a salt thereof.

45. The composition for use of any one of claims 33-35, wherein the composition comprises an effective amount of osimertinib, binimetinib or a salt thereof, and palbociclib or a salt thereof

46. The composition for use of any one of claims 33-45, wherein the cancer has a KRAS G12 mutation or a KRAS G13 mutation.

47. The composition for use of claim 46, wherein the KRAS G12 mutation is G12A, G12V, G12C, or G12D mutation.

48. The composition for use of claim 46, wherein the KRAS G13 mutation is G13D mutation.

49. The composition for use of any one of claims 33-35, wherein the composition comprises osimertinib or a salt thereof of about 40-80 mg.

50. The composition for use of any one of claims 33-35, wherein the composition comprises cobimetinib or a salt thereof of about 20-60 mg.

51. The composition for use of any one of claims 33-35, wherein the composition comprises palbociclib or a salt thereof of about 75-125 mg.

52. A kit for treating or delaying progression of colorectal cancer in a subject, wherein the kit comprises (a) an epidermal growth factor receptor (EGFR) inhibitor; (b) a mitogen-activated protein kinase (MEK) 1/2 inhibitor; and (c) a cyclin dependent kinase (CDK) 4/6 inhibitor.

53. The kit of claim 52, wherein the kit is for use according to the method of any one of claims 1-32.

54. The kit of claim 52 or 53, wherein the kit does not comprise a KRAS inhibitor.

55. The kit of any one of claims 52-54, wherein the EGFR inhibitor is osimertinib or a salt thereof, lapatinib or a salt thereof, or cetuximab, wherein the MEK 1/2 inhibitor is cobimetinib or a salt thereof, trametinib or a salt thereof, binimetinib or a salt thereof, or TAK-733 or a salt thereof, and wherein the CDK 4/6 inhibitor is palbociclib or a salt thereof, or abemaciclib or a salt thereof.

56. The kit of any one of claims 52-54, wherein the kit comprises an effective amount of osimertinib or a salt thereof, cobimetinib or a salt thereof, and palbociclib or a salt thereof.

57. The kit of any one of claims 52-54, wherein the kit comprises an effective amount of osimertinib or a salt thereof, TAK-733 or a salt thereof, and palbociclib or a salt thereof.

58. The kit of any one of claims 52-54, wherein the kit comprises an effective amount of osimertinib or a salt thereof, trametinib or a salt thereof, and palbociclib or a salt thereof.

59. The kit of any one of claims 52-54, wherein the kit comprises an effective amount of cetuximab, cobimetinib or a salt thereof, and palbociclib or a salt thereof.

60. The kit of any one of claims 52-54, wherein the kit comprises an effective amount of cetuximab, TAK-733 or a salt thereof, and palbociclib or a salt thereof.

61. The kit of any one of claims 52-54, wherein the kit comprises an effective amount of cetuximab, trametinib or a salt thereof, and palbociclib or a salt thereof.

62. The kit of any one of claims 52-53, wherein the kit comprises an effective amount of cetuximab, cobimetinib or a salt thereof, and abemaciclib or a salt thereof.

63. The kit of any one of claims 52-53, wherein the kit comprises an effective amount of cetuximab, binimetinib or a salt thereof, and palbociclib or a salt thereof.

64. The kit of any one of claims 52-53, wherein the kit comprises an effective amount of osimertinib, binimetinib or a salt thereof, and palbociclib or a salt thereof.
